SEQUENCE LISTING (365 sequences)

SEQ ID NO: 1 - protein, 5 aa, synthetic construct
  REGION 1..5: note = Description of Artificial Sequence: Synthetic peptide
NYNMH

SEQ ID NO: 2 - protein, 17 aa, synthetic construct
  REGION 1..17: note = Description of Artificial Sequence: Synthetic peptide
TIYPGNDDTSYNQKFKD

SEQ ID NO: 3 - protein, 8 aa, synthetic construct
  REGION 1..8: note = Description of Artificial Sequence: Synthetic peptide
GGYRAMDY

SEQ ID NO: 4 - protein, 16 aa, synthetic construct
  REGION 1..16: note = Description of Artificial Sequence: Synthetic peptide
RSSQSIVYSNGNTYLG

SEQ ID NO: 5 - protein, 7 aa, synthetic construct
  REGION 1..7: note = Description of Artificial Sequence: Synthetic peptide
KVSNRFS

SEQ ID NO: 6 - protein, 9 aa, synthetic construct
  REGION 1..9: note = Description of Artificial Sequence: Synthetic peptide
FQGSHVPYT

SEQ ID NO: 7 - protein, 5 aa, synthetic construct
  REGION 1..5: note = Description of Artificial Sequence: Synthetic peptide
DYYIN

SEQ ID NO: 8 - protein, 17 aa, synthetic construct
  REGION 1..17: note = Description of Artificial Sequence: Synthetic peptide
RIYPGIGNTYYNKKFKG

SEQ ID NO: 9 - protein, 9 aa, synthetic construct
  REGION 1..9: note = Description of Artificial Sequence: Synthetic peptide
GHYGRGMDY

SEQ ID NO: 10 - protein, 17 aa, synthetic construct
  REGION 1..17: note = Description of Artificial Sequence: Synthetic peptide
KSSQSLLNSIDQKNYLA

SEQ ID NO: 11 - protein, 7 aa, synthetic construct
  REGION 1..7: note = Description of Artificial Sequence: Synthetic peptide
FASTKES

SEQ ID NO: 12 - protein, 9 aa, synthetic construct
  REGION 1..9: note = Description of Artificial Sequence: Synthetic peptide
QQHYSTPWT

SEQ ID NO: 13 - protein, 5 aa, synthetic construct
  REGION 1..5: note = Description of Artificial Sequence: Synthetic peptide
RAWMN

SEQ ID NO: 14 - protein, 19 aa, synthetic construct
  REGION 1..19: note = Description of Artificial Sequence: Synthetic peptide
RIKRKTDGETTDYAAPVKG

SEQ ID NO: 15 - protein, 7 aa, synthetic construct
  REGION 1..7: note = Description of Artificial Sequence: Synthetic peptide
SNRAFDI

SEQ ID NO: 16 - protein, 17 aa, synthetic construct
  REGION 1..17: note = Description of Artificial Sequence: Synthetic peptide
KSSQSVLYAGNNRNYLA

SEQ ID NO: 17 - protein, 7 aa, synthetic construct
  REGION 1..7: note = Description of Artificial Sequence: Synthetic peptide
QASTRAS

SEQ ID NO: 18 - protein, 9 aa, synthetic construct
  REGION 1..9: note = Description of Artificial Sequence: Synthetic peptide
QQYYTPPLA

SEQ ID NO: 19 - protein, 6 aa, synthetic construct
  REGION 1..6: note = Description of Artificial Sequence: Synthetic peptide
SYYWSW

SEQ ID NO: 20 - protein, 16 aa, synthetic construct
  REGION 1..16: note = Description of Artificial Sequence: Synthetic peptide
YIYYSGSTNYNPSLKS

SEQ ID NO: 21 - protein, 7 aa, synthetic construct
  REGION 1..7: note = Description of Artificial Sequence: Synthetic peptide
GKTGSAA

SEQ ID NO: 22 - protein, 11 aa, synthetic construct
  REGION 1..11: note = Description of Artificial Sequence: Synthetic peptide
RASQGISRWLA

SEQ ID NO: 23 - protein, 7 aa, synthetic construct
  REGION 1..7: note = Description of Artificial Sequence: Synthetic peptide
AASSLQS

SEQ ID NO: 24 - protein, 9 aa, synthetic construct
  REGION 1..9: note = Description of Artificial Sequence: Synthetic peptide
QQTVSFPIT

SEQ ID NO: 25 - protein, 8 aa, synthetic construct
  REGION 1..8: note = Description of Artificial Sequence: Synthetic peptide
GYTFTNYN

SEQ ID NO: 26 - protein, 8 aa, synthetic construct
  REGION 1..8: note = Description of Artificial Sequence: Synthetic peptide
IYPGNDDT

SEQ ID NO: 27 - protein, 10 aa, synthetic construct
  REGION 1..10: note = Description of Artificial Sequence: Synthetic peptide
ARGGYRAMDY

SEQ ID NO: 28 - protein, 11 aa, synthetic construct
  REGION 1..11: note = Description of Artificial Sequence: Synthetic peptide
QSIVYSNGNTY

SEQ ID NO: 29 - skipped

SEQ ID NO: 30 - protein, 8 aa, synthetic construct
  REGION 1..8: note = Description of Artificial Sequence: Synthetic peptide
GYSFTDYY

SEQ ID NO: 31 - protein, 8 aa, synthetic construct
  REGION 1..8: note = Description of Artificial Sequence: Synthetic peptide
IYPGIGNT

SEQ ID NO: 32 - protein, 11 aa, synthetic construct
  REGION 1..11: note = Description of Artificial Sequence: Synthetic peptide
ARGHYGRGMDY

SEQ ID NO: 33 - protein, 12 aa, synthetic construct
  REGION 1..12: note = Description of Artificial Sequence: Synthetic peptide
QSLLNSIDQKNY

SEQ ID NO: 34 - skipped

SEQ ID NO: 35 - protein, 8 aa, synthetic construct
  REGION 1..8: note = Description of Artificial Sequence: Synthetic peptide
GLTFERAW

SEQ ID NO: 36 - protein, 10 aa, synthetic construct
  REGION 1..10: note = Description of Artificial Sequence: Synthetic peptide
IKRKTDGETT

SEQ ID NO: 37 - protein, 9 aa, synthetic construct
  REGION 1..9: note = Description of Artificial Sequence: Synthetic peptide
AGSNRAFDI

SEQ ID NO: 38 - protein, 12 aa, synthetic construct
  REGION 1..12: note = Description of Artificial Sequence: Synthetic peptide
QSVLYAGNNRNY

SEQ ID NO: 39 - skipped

SEQ ID NO: 40 - protein, 8 aa, synthetic construct
  REGION 1..8: note = Description of Artificial Sequence: Synthetic peptide
GGSISSYY

SEQ ID NO: 41 - protein, 7 aa, synthetic construct
  REGION 1..7: note = Description of Artificial Sequence: Synthetic peptide
IYYSGST

SEQ ID NO: 42 - protein, 9 aa, synthetic construct
  REGION 1..9: note = Description of Artificial Sequence: Synthetic peptide
ARGKTGSAA

SEQ ID NO: 43 - protein, 6 aa, synthetic construct
  REGION 1..6: note = Description of Artificial Sequence: Synthetic peptide
QGISRW

SEQ ID NO: 44 - skipped

SEQ ID NO: 45 - protein, 7 aa, synthetic construct
  REGION 1..7: note = Description of Artificial Sequence: Synthetic peptide
GYTFTNY

SEQ ID NO: 46 - protein, 4 aa, synthetic construct
  REGION 1..4: note = Description of Artificial Sequence: Synthetic peptide
PGND

SEQ ID NO: 47 - protein, 6 aa, synthetic construct
  REGION 1..6: note = Description of Artificial Sequence: Synthetic peptide
GYRAMD

SEQ ID NO: 48 - protein, 12 aa, synthetic construct
  REGION 1..12: note = Description of Artificial Sequence: Synthetic peptide
SQSIVYSNGNTY

SEQ ID NO: 49 - protein, 6 aa, synthetic construct
  REGION 1..6: note = Description of Artificial Sequence: Synthetic peptide
GSHVPY

SEQ ID NO: 50 - protein, 7 aa, synthetic construct
  REGION 1..7: note = Description of Artificial Sequence: Synthetic peptide
GYSFTDY

SEQ ID NO: 51 - protein, 4 aa, synthetic construct
  REGION 1..4: note = Description of Artificial Sequence: Synthetic peptide
PGIG

SEQ ID NO: 52 - protein, 7 aa, synthetic construct
  REGION 1..7: note = Description of Artificial Sequence: Synthetic peptide
HYGRGMD

SEQ ID NO: 53 - protein, 13 aa, synthetic construct
  REGION 1..13: note = Description of Artificial Sequence: Synthetic peptide
SQSLLNSIDQKNY

SEQ ID NO: 54 - protein, 6 aa, synthetic construct
  REGION 1..6: note = Description of Artificial Sequence: Synthetic peptide
HYSTPW

SEQ ID NO: 55 - protein, 7 aa, synthetic construct
  REGION 1..7: note = Description of Artificial Sequence: Synthetic peptide
GLTFERA

SEQ ID NO: 56 - protein, 6 aa, synthetic construct
  REGION 1..6: note = Description of Artificial Sequence: Synthetic peptide
RKTDGE

SEQ ID NO: 57 - protein, 5 aa, synthetic construct
  REGION 1..5: note = Description of Artificial Sequence: Synthetic peptide
NRAFD

SEQ ID NO: 58 - protein, 13 aa, synthetic construct
  REGION 1..13: note = Description of Artificial Sequence: Synthetic peptide
SQSVLYAGNNRNY

SEQ ID NO: 59 - protein, 6 aa, synthetic construct
  REGION 1..6: note = Description of Artificial Sequence: Synthetic peptide
YYTPPL

SEQ ID NO: 60 - protein, 7 aa, synthetic construct
  REGION 1..7: note = Description of Artificial Sequence: Synthetic peptide
GGSISSY

SEQ ID NO: 61 - skipped

SEQ ID NO: 62 - protein, 5 aa, synthetic construct
  REGION 1..5: note = Description of Artificial Sequence: Synthetic peptide
KTGSA

SEQ ID NO: 63 - protein, 7 aa, synthetic construct
  REGION 1..7: note = Description of Artificial Sequence: Synthetic peptide
SQGISRW

SEQ ID NO: 64 - protein, 6 aa, synthetic construct
  REGION 1..6: note = Description of Artificial Sequence: Synthetic peptide
TVSFPI

SEQ ID NO: 65 - protein, 10 aa, synthetic construct
  REGION 1..10: note = Description of Artificial Sequence: Synthetic peptide
ASGYTFTNYN

SEQ ID NO: 66 - protein, 17 aa, synthetic construct
  REGION 1..17: note = Description of Artificial Sequence: Synthetic peptide
IYPGNDDTSYNQKFKDR

SEQ ID NO: 67 - protein, 7 aa, synthetic construct
  REGION 1..7: note = Description of Artificial Sequence: Synthetic peptide
GGYRAMD

SEQ ID NO: 68 - protein, 13 aa, synthetic construct
  REGION 1..13: note = Description of Artificial Sequence: Synthetic peptide
SSQSIVYSNGNTY

SEQ ID NO: 69 - protein, 12 aa, synthetic construct
  REGION 1..12: note = Description of Artificial Sequence: Synthetic peptide
KVSNRFSGVPDR

SEQ ID NO: 70 - protein, 10 aa, synthetic construct
  REGION 1..10: note = Description of Artificial Sequence: Synthetic peptide
ASGYSFTDYY

SEQ ID NO: 71 - protein, 17 aa, synthetic construct
  REGION 1..17: note = Description of Artificial Sequence: Synthetic peptide
IYPGIGNTYYNKKFKGR

SEQ ID NO: 72 - protein, 8 aa, synthetic construct
  REGION 1..8: note = Description of Artificial Sequence: Synthetic peptide
GHYGRGMD

SEQ ID NO: 73 - protein, 14 aa, synthetic construct
  REGION 1..14: note = Description of Artificial Sequence: Synthetic peptide
SSQSLLNSIDQKNY

SEQ ID NO: 74 - protein, 12 aa, synthetic construct
  REGION 1..12: note = Description of Artificial Sequence: Synthetic peptide
FASTKESGVPDR

SEQ ID NO: 75 - protein, 10 aa, synthetic construct
  REGION 1..10: note = Description of Artificial Sequence: Synthetic peptide
ASGLTFERAW

SEQ ID NO: 76 - protein, 19 aa, synthetic construct
  REGION 1..19: note = Description of Artificial Sequence: Synthetic peptide
IKRKTDGETTDYAAPVKGR

SEQ ID NO: 77 - protein, 6 aa, synthetic construct
  REGION 1..6: note = Description of Artificial Sequence: Synthetic peptide
SNRAFD

SEQ ID NO: 78 - protein, 14 aa, synthetic construct
  REGION 1..14: note = Description of Artificial Sequence: Synthetic peptide
SSQSVLYAGNNRNY

SEQ ID NO: 79 - protein, 12 aa, synthetic construct
  REGION 1..12: note = Description of Artificial Sequence: Synthetic peptide
QASTRASGVPDR

SEQ ID NO: 80 - protein, 10 aa, synthetic construct
  REGION 1..10: note = Description of Artificial Sequence: Synthetic peptide
VSGGSISSYY

SEQ ID NO: 81 - protein, 16 aa, synthetic construct
  REGION 1..16: note = Description of Artificial Sequence: Synthetic peptide
IYYSGSTNYNPSLKSR

SEQ ID NO: 82 - protein, 6 aa, synthetic construct
  REGION 1..6: note = Description of Artificial Sequence: Synthetic peptide
GKTGSA

SEQ ID NO: 83 - protein, 8 aa, synthetic construct
  REGION 1..8: note = Description of Artificial Sequence: Synthetic peptide
ASQGISRW

SEQ ID NO: 84 - protein, 12 aa, synthetic construct
  REGION 1..12: note = Description of Artificial Sequence: Synthetic peptide
AASSLQSGVPSR

SEQ ID NO: 85 - protein, 117 aa, synthetic construct
  REGION 1..117: note = Description of Artificial Sequence: Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 86 - protein, 112 aa, synthetic construct
  REGION 1..112: note = Description of Artificial Sequence: Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 87 - protein, 118 aa, synthetic construct
  REGION 1..118: note = Description of Artificial Sequence: Synthetic polypeptide
QVQLVQSGAEVKKPGASVKVSCKASGYSFTDYYINWVRQAPGQGLEWMGRIYPGIGNTYYNKKFKGRVTITRDTSASTAYMELSSLRSEDTAVYYCARGHYGRGMDYWGQGTLVTVSS

SEQ ID NO: 88 - protein, 113 aa, synthetic construct
  REGION 1..113: note = Description of Artificial Sequence: Synthetic polypeptide
DIVMTQSPDSLAVSLGERATINCKSSQSLLNSIDQKNYLAWYQQKPGQPPKLLIYFASTKESGVPDRFSGSGSGTDFTLTISGLQAEDVAVYFCQQHYSTPWTFGGGTKVEIR

SEQ ID NO: 89 - protein, 118 aa, synthetic construct
  REGION 1..118: note = Description of Artificial Sequence: Synthetic polypeptide
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSS

SEQ ID NO: 90 - protein, 113 aa, synthetic construct
  REGION 1..113: note = Description of Artificial Sequence: Synthetic polypeptide
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 91 - protein, 115 aa, synthetic construct
  REGION 1..115: note = Description of Artificial Sequence: Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGKTGSAAWGQGTLVTVSS

SEQ ID NO: 92 - protein, 107 aa, synthetic construct
  REGION 1..107: note = Description of Artificial Sequence: Synthetic polypeptide
DIQMTQSPSSVSASVGDRVTITCRASQGISRWLAWYQQKPGKAPKLLIYAASSLQSGVPSRFSGSGSGTDFTLTISSLQPEDFATYYCQQTVSFPITFGGGTKVEIK

SEQ ID NO: 93 - protein, 5 aa, synthetic construct
  REGION 1..5: note = Description of Artificial Sequence: Synthetic peptide
SYWIT

SEQ ID NO: 94 - protein, 17 aa, synthetic construct
  REGION 1..17: note = Description of Artificial Sequence: Synthetic peptide
DIYPGSGSTNHIEKFKS

SEQ ID NO: 95 - protein, 11 aa, synthetic construct
  REGION 1..11: note = Description of Artificial Sequence: Synthetic peptide
GYGSSYGYFDY

SEQ ID NO: 96 - protein, 11 aa, synthetic construct
  REGION 1..11: note = Description of Artificial Sequence: Synthetic peptide
RASENIYSYLA

SEQ ID NO: 97 - protein, 7 aa, synthetic construct
  REGION 1..7: note = Description of Artificial Sequence: Synthetic peptide
TAKTLAE

SEQ ID NO: 98 - protein, 9 aa, synthetic construct
  REGION 1..9: note = Description of Artificial Sequence: Synthetic peptide
QHQYGPPFT

SEQ ID NO: 99 - protein, 5 aa, synthetic construct
  REGION 1..5: note = Description of Artificial Sequence: Synthetic peptide
SYWMH

SEQ ID NO: 100 - protein, 17 aa, synthetic construct
  REGION 1..17: note = Description of Artificial Sequence: Synthetic peptide
NIDPSDSDTHYNQKFKD

SEQ ID NO: 101 - protein, 10 aa, synthetic construct
  REGION 1..10: note = Description of Artificial Sequence: Synthetic peptide
GYSKYYAMDY

SEQ ID NO: 102 - protein, 16 aa, synthetic construct
  REGION 1..16: note = Description of Artificial Sequence: Synthetic peptide
RSSQSIVHSYGNTYLE

SEQ ID NO: 103 - protein, 7 aa, synthetic construct
  REGION 1..7: note = Description of Artificial Sequence: Synthetic peptide
KVSNRFS

SEQ ID NO: 104 - protein, 9 aa, synthetic construct
  REGION 1..9: note = Description of Artificial Sequence: Synthetic peptide
FQGSHVPYT

SEQ ID NO: 105 - protein, 11 aa, synthetic construct
  REGION 1..11: note = Description of Artificial Sequence: Synthetic peptide
YGNYGENAMDY

SEQ ID NO: 106 - protein, 9 aa, synthetic construct
  REGION 1..9: note = Description of Artificial Sequence: Synthetic peptide
FQGSHVPFT

SEQ ID NO: 107 - protein, 5 aa, synthetic construct
  REGION 1..5: note = Description of Artificial Sequence: Synthetic peptide
DYYIH

SEQ ID NO: 108 - protein, 17 aa, synthetic construct
  REGION 1..17: note = Description of Artificial Sequence: Synthetic peptide
RIDPEDGETKYAPKFQG

SEQ ID NO: 109 - protein, 5 aa, synthetic construct
  REGION 1..5: note = Description of Artificial Sequence: Synthetic peptide
GGFAY

SEQ ID NO: 110 - protein, 11 aa, synthetic construct
  REGION 1..11: note = Description of Artificial Sequence: Synthetic peptide
ASSSVSSSYLY

SEQ ID NO: 111 - protein, 7 aa, synthetic construct
  REGION 1..7: note = Description of Artificial Sequence: Synthetic peptide
STSNLAS

SEQ ID NO: 112 - protein, 9 aa, synthetic construct
  REGION 1..9: note = Description of Artificial Sequence: Synthetic peptide
HQWSSHPYT

SEQ ID NO: 113 - protein, 5 aa, synthetic construct
  REGION 1..5: note = Description of Artificial Sequence: Synthetic peptide
SYWVH

SEQ ID NO: 114 - protein, 17 aa, synthetic construct
  REGION 1..17: note = Description of Artificial Sequence: Synthetic peptide
NIDPSDSDTHYSPSFQG

SEQ ID NO: 115 - protein, 11 aa, synthetic construct
  REGION 1..11: note = Description of Artificial Sequence: Synthetic peptide
GGTGTLAYFAY

SEQ ID NO: 116 - protein, 16 aa, synthetic construct
  REGION 1..16: note = Description of Artificial Sequence: Synthetic peptide
RSSQSLVHSYGNTYLY

SEQ ID NO: 117 - protein, 7 aa, synthetic construct
  REGION 1..7: note = Description of Artificial Sequence: Synthetic peptide
RVSNRFS

SEQ ID NO: 118 - protein, 9 aa, synthetic construct
  REGION 1..9: note = Description of Artificial Sequence: Synthetic peptide
FQGTHVPYT

SEQ ID NO: 119 - protein, 5 aa, synthetic construct
  REGION 1..5: note = Description of Artificial Sequence: Synthetic peptide
GYGIS

SEQ ID NO: 120 - protein, 17 aa, synthetic construct
  REGION 1..17: note = Description of Artificial Sequence: Synthetic peptide
WISAYGGETNYAQKLQG

SEQ ID NO: 121 - protein, 11 aa, synthetic construct
  REGION 1..11: note = Description of Artificial Sequence: Synthetic peptide
EAGSSWYDFDL

SEQ ID NO: 122 - protein, 11 aa, synthetic construct
  REGION 1..11: note = Description of Artificial Sequence: Synthetic peptide
RASQGISSWLA

SEQ ID NO: 123 - protein, 7 aa, synthetic construct
  REGION 1..7: note = Description of Artificial Sequence: Synthetic peptide
AASNLQS

SEQ ID NO: 124 - protein, 9 aa, synthetic construct
  REGION 1..9: note = Description of Artificial Sequence: Synthetic peptide
QQGASFPIT

SEQ ID NO: 125 - protein, 8 aa, synthetic construct
  REGION 1..8: note = Description of Artificial Sequence: Synthetic peptide
GYTFTSYW

SEQ ID NO: 126 - protein, 8 aa, synthetic construct
  REGION 1..8: note = Description of Artificial Sequence: Synthetic peptide
IYPGSGST

SEQ ID NO: 127 - protein, 13 aa, synthetic construct
  REGION 1..13: note = Description of Artificial Sequence: Synthetic peptide
ATGYGSSYGYFDY

SEQ ID NO: 128 - protein, 6 aa, synthetic construct
  REGION 1..6: note = Description of Artificial Sequence: Synthetic peptide
ENIYSY

SEQ ID NO: 129 - skipped

SEQ ID NO: 130 - protein, 8 aa, synthetic construct
  REGION 1..8: note = Description of Artificial Sequence: Synthetic peptide
IDPSDSDT

SEQ ID NO: 131 - protein, 12 aa, synthetic construct
  REGION 1..12: note = Description of Artificial Sequence: Synthetic peptide
ARGYSKYYAMDY

SEQ ID NO: 132 - protein, 11 aa, synthetic construct
  REGION 1..11: note = Description of Artificial Sequence: Synthetic peptide
QSIVHSYGNTY

SEQ ID NO: 133 - protein, 13 aa, synthetic construct
  REGION 1..13: note = Description of Artificial Sequence: Synthetic peptide
ASYGNYGENAMDY

SEQ ID NO: 134 - protein, 8 aa, synthetic construct
  REGION 1..8: note = Description of Artificial Sequence: Synthetic peptide
GFNIKDYY

SEQ ID NO: 135 - protein, 8 aa, synthetic construct
  REGION 1..8: note = Description of Artificial Sequence: Synthetic peptide
IDPEDGET

SEQ ID NO: 136 - protein, 7 aa, synthetic construct
  REGION 1..7: note = Description of Artificial Sequence: Synthetic peptide
AKGGFAY

SEQ ID NO: 137 - protein, 7 aa, synthetic construct
  REGION 1..7: note = Description of Artificial Sequence: Synthetic peptide
SSVSSSY

SEQ ID NO: 138 - skipped

SEQ ID NO: 139 - protein, 8 aa, synthetic construct
  REGION 1..8: note = Description of Artificial Sequence: Synthetic peptide
GYSFTSYW

SEQ ID NO: 140 - protein, 13 aa, synthetic construct
  REGION 1..13: note = Description of Artificial Sequence: Synthetic peptide
VRGGTGTLAYFAY

SEQ ID NO: 141 - protein, 11 aa, synthetic construct
  REGION 1..11: note = Description of Artificial Sequence: Synthetic peptide
QSLVHSYGNTY

SEQ ID NO: 142 - skipped

SEQ ID NO: 143 - protein, 8 aa, synthetic construct
  REGION 1..8: note = Description of Artificial Sequence: Synthetic peptide
GYTFRGYG

SEQ ID NO: 144 - protein, 8 aa, synthetic construct
  REGION 1..8: note = Description of Artificial Sequence: Synthetic peptide
ISAYGGET

SEQ ID NO: 145 - protein, 13 aa, synthetic construct
  REGION 1..13: note = Description of Artificial Sequence: Synthetic peptide
AREAGSSWYDFDL

SEQ ID NO: 146 - protein, 6 aa, synthetic construct
  REGION 1..6: note = Description of Artificial Sequence: Synthetic peptide
QGISSW

SEQ ID NO: 147 - protein, 7 aa, synthetic construct
  REGION 1..7: note = Description of Artificial Sequence: Synthetic peptide
GYTFTSY

SEQ ID NO: 148 - protein, 4 aa, synthetic construct
  REGION 1..4: note = Description of Artificial Sequence: Synthetic peptide
PGSG

SEQ ID NO: 149 - protein, 9 aa, synthetic construct
  REGION 1..9: note = Description of Artificial Sequence: Synthetic peptide
YGSSYGYFD

SEQ ID NO: 150 - protein, 7 aa, synthetic construct
  REGION 1..7: note = Description of Artificial Sequence: Synthetic peptide
SENIYSY

SEQ ID NO: 151 - protein, 6 aa, synthetic construct
  REGION 1..6: note = Description of Artificial Sequence: Synthetic peptide
QYGPPF

SEQ ID NO: 152 - protein, 4 aa, synthetic construct
  REGION 1..4: note = Description of Artificial Sequence: Synthetic peptide
PSDS

SEQ ID NO: 153 - protein, 8 aa, synthetic construct
  REGION 1..8: note = Description of Artificial Sequence: Synthetic peptide
YSKYYAMD

SEQ ID NO: 154 - protein, 12 aa, synthetic construct
  REGION 1..12: note = Description of Artificial Sequence: Synthetic peptide
SQSIVHSYGNTY

SEQ ID NO: 155 - protein, 6 aa, synthetic construct
  REGION 1..6: note = Description of Artificial Sequence: Synthetic peptide
GSHVPY

SEQ ID NO: 156 - protein, 9 aa, synthetic construct
  REGION 1..9: note = Description of Artificial Sequence: Synthetic peptide
GNYGENAMD

SEQ ID NO: 157 - protein, 6 aa, synthetic construct
  REGION 1..6: note = Description of Artificial Sequence: Synthetic peptide
GSHVPF

SEQ ID NO: 158 - protein, 7 aa, synthetic construct
  REGION 1..7: note = Description of Artificial Sequence: Synthetic peptide
GFNIKDY

SEQ ID NO: 159 - protein, 4 aa, synthetic construct
  REGION 1..4: note = Description of Artificial Sequence: Synthetic peptide
PEDG

SEQ ID NO: 160 - skipped

SEQ ID NO: 161 - protein, 8 aa, synthetic construct
  REGION 1..8: note = Description of Artificial Sequence: Synthetic peptide
SSSVSSSY

SEQ ID NO: 162 - protein, 6 aa, synthetic construct
  REGION 1..6: note = Description of Artificial Sequence: Synthetic peptide
WSSHPY

SEQ ID NO: 163 - protein, 7 aa, synthetic construct
  REGION 1..7: note = Description of Artificial Sequence: Synthetic peptide
GYSFTSY

SEQ ID NO: 164 - protein, 9 aa, synthetic construct
  REGION 1..9: note = Description of Artificial Sequence: Synthetic peptide
GTGTLAYFA

SEQ ID NO: 165 - protein, 12 aa, synthetic construct
  REGION 1..12: note = Description of Artificial Sequence: Synthetic peptide
SQSLVHSYGNTY

SEQ ID NO: 166 - protein, 6 aa, synthetic construct
  REGION 1..6: note = Description of Artificial Sequence: Synthetic peptide
GTHVPY

SEQ ID NO: 167 - protein, 7 aa, synthetic construct
  REGION 1..7: note = Description of Artificial Sequence: Synthetic peptide
GYTFRGY

SEQ ID NO: 168 - protein, 4 aa, synthetic construct
  REGION 1..4: note = Description of Artificial Sequence: Synthetic peptide
AYGG

SEQ ID NO: 169 - protein, 9 aa, synthetic construct
  REGION 1..9: note = Description of Artificial Sequence: Synthetic peptide
AGSSWYDFD

SEQ ID NO: 170 - protein, 7 aa, synthetic construct
  REGION 1..7: note = Description of Artificial Sequence: Synthetic peptide
SQGISSW

SEQ ID NO: 171 - protein, 6 aa, synthetic construct
  REGION 1..6: note = Description of Artificial Sequence: Synthetic peptide
GASFPI

SEQ ID NO: 172 - protein, 10 aa, synthetic construct
  REGION 1..10: note = Description of Artificial Sequence: Synthetic peptide
ASGYTFTSYW

SEQ ID NO: 173 - protein, 17 aa, synthetic construct
  REGION 1..17: note = Description of Artificial Sequence: Synthetic peptide
IYPGSGSTNHIEKFKSK

SEQ ID NO: 174 - protein, 10 aa, synthetic construct
  REGION 1..10: note = Description of Artificial Sequence: Synthetic peptide
GYGSSYGYFD

SEQ ID NO: 175 - protein, 8 aa, synthetic construct
  REGION 1..8: note = Description of Artificial Sequence: Synthetic peptide
ASENIYSY

SEQ ID NO: 176 - protein, 12 aa, synthetic construct
  REGION 1..12: note = Description of Artificial Sequence: Synthetic peptide
TAKTLAEGVPSR

SEQ ID NO: 177 - protein, 17 aa, synthetic construct
  REGION 1..17: note = Description of Artificial Sequence: Synthetic peptide
IDPSDSDTHYNQKFKDR

SEQ ID NO: 178 - protein, 9 aa, synthetic construct
  REGION 1..9: note = Description of Artificial Sequence: Synthetic peptide
GYSKYYAMD

SEQ ID NO: 179 - protein, 13 aa, synthetic construct
  REGION 1..13: note = Description of Artificial Sequence: Synthetic peptide
SSQSIVHSYGNTY

SEQ ID NO: 180 - protein, 12 aa, synthetic construct
  REGION 1..12: note = Description of Artificial Sequence: Synthetic peptide
KVSNRFSGVPDR

SEQ ID NO: 181 - protein, 17 aa, synthetic construct
  REGION 1..17: note = Description of Artificial Sequence: Synthetic peptide
IDPSDSDTHYNQKFKDK

SEQ ID NO: 182 - protein, 10 aa, synthetic construct
  REGION 1..10: note = Description of Artificial Sequence: Synthetic peptide
YGNYGENAMD

SEQ ID NO: 183 - protein, 10 aa, synthetic construct
  REGION 1..10: note = Description of Artificial Sequence: Synthetic peptide
ASGFNIKDYY

SEQ ID NO: 184 - protein, 17 aa, synthetic construct
  REGION 1..17: note = Description of Artificial Sequence: Synthetic peptide
IDPEDGETKYAPKFQGK

SEQ ID NO: 185 - protein, 4 aa, synthetic construct
  REGION 1..4: note = Description of Artificial Sequence: Synthetic peptide
GGFA

SEQ ID NO: 186 - protein, 9 aa, synthetic construct
  REGION 1..9: note = Description of Artificial Sequence: Synthetic peptide
ASSSVSSSY

SEQ ID NO: 187 - protein, 12 aa, synthetic construct
  REGION 1..12: note = Description of Artificial Sequence: Synthetic peptide
STSNLASGVPAR

SEQ ID NO: 188 - protein, 10 aa, synthetic construct
  REGION 1..10: note = Description of Artificial Sequence: Synthetic peptide
ASGYSFTSYW

SEQ ID NO: 189 - protein, 17 aa, synthetic construct
  REGION 1..17: note = Description of Artificial Sequence: Synthetic peptide
IDPSDSDTHYSPSFQGH

SEQ ID NO: 190 - protein, 10 aa, synthetic construct
  REGION 1..10: note = Description of Artificial Sequence: Synthetic peptide
GGTGTLAYFA

SEQ ID NO: 191 - protein, 13 aa, synthetic construct
  REGION 1..13: note = Description of Artificial Sequence: Synthetic peptide
SSQSLVHSYGNTY

SEQ ID NO: 192 - protein, 12 aa, synthetic construct
  REGION 1..12: note = Description of Artificial Sequence: Synthetic peptide
RVSNRFSGVPDR

SEQ ID NO: 193 - protein, 10 aa, synthetic construct
  REGION 1..10: note = Description of Artificial Sequence: Synthetic peptide
ASGYTFRGYG

SEQ ID NO: 194 - protein, 17 aa, synthetic construct
  REGION 1..17: note = Description of Artificial Sequence: Synthetic peptide
ISAYGGETNYAQKLQGR

SEQ ID NO: 195 - protein, 10 aa, synthetic construct
  REGION 1..10: note = Description of Artificial Sequence: Synthetic peptide
EAGSSWYDFD

SEQ ID NO: 196 - protein, 8 aa, synthetic construct
  REGION 1..8: note = Description of Artificial Sequence: Synthetic peptide
ASQGISSW

SEQ ID NO: 197 - protein, 12 aa, synthetic construct
  REGION 1..12: note = Description of Artificial Sequence: Synthetic peptide
AASNLQSGVPSR

SEQ ID NO: 198 - protein, 120 aa, synthetic construct
  REGION 1..120: note = Description of Artificial Sequence: Synthetic polypeptide
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSS

SEQ ID NO: 199 - protein, 107 aa, synthetic construct
  REGION 1..107: note = Description of Artificial Sequence: Synthetic polypeptide
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIK

SEQ ID NO: 200 - protein, 119 aa, synthetic construct
  REGION 1..119: note = Description of Artificial Sequence: Synthetic polypeptide
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSS

SEQ ID NO: 201 - protein, 112 aa, synthetic construct
  REGION 1..112: note = Description of Artificial Sequence: Synthetic polypeptide
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIK

SEQ ID NO: 202 - protein, 120 aa, synthetic construct
  REGION 1..120: note = Description of Artificial Sequence: Synthetic polypeptide
QVKLQESGAELVRPGSSVKLSCKASGYTFTSYWMHWVKQRPIQGLEWIGNIDPSDSDTHYNQKFKDKATLTVDNSSSTAYMQLSSLTSEDSAVYYCASYGNYGENAMDYWGQGTSVTVSS

SEQ ID NO: 203 - protein, 112 aa, synthetic construct
  REGION 1..112: note = Description of Artificial Sequence: Synthetic polypeptide
DILMTQTPLSLPVSLGDQASISCRSSQSIVHSYGNTYLEWYLQKPGQSPKLLIYKVSNRFSGVPDRFSGSGSGTDFTLKISRVEAEDLGVYYCFQGSHVPFTFGSGTKLEIK

SEQ ID NO: 204 - protein, 114 aa, synthetic construct
  REGION 1..114: note = Description of Artificial Sequence: Synthetic polypeptide
EVQLQQSGAELVKPGASVKLSCTASGFNIKDYYIHWVKQRTEQGLEWIGRIDPEDGETKYAPKFQGKATITADTSSNTAYLQLNSLTSEDTAVYSCAKGGFAYWGQGTLVTVSA

SEQ ID NO: 205 - protein, 108 aa, synthetic construct
  REGION 1..108: note = Description of Artificial Sequence: Synthetic polypeptide
QIVLTQSPAIMSASPGEKVTLTCSASSSVSSSYLYWYQQKPGSSPKLWIYSTSNLASGVPARFSGSGSGTSYSLTISSMEAEDAASYFCHQWSSHPYTFGGGTKLEIK

SEQ ID NO: 206 - protein, 120 aa, synthetic construct
  REGION 1..120: note = Description of Artificial Sequence: Synthetic polypeptide
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 207 - protein, 112 aa, synthetic construct
  REGION 1..112: note = Description of Artificial Sequence: Synthetic polypeptide
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 208 - protein, 120 aa, synthetic construct
  REGION 1..120: note = Description of Artificial Sequence: Synthetic polypeptide
QVQLVQSGAEVKKPGASVKVSCKASGYTFRGYGISWVRQAPGQGLEWMGWISAYGGETNYAQKLQGRVTMTTDTSTSTAYMELRSLRSDDTAVYYCAREAGSSWYDFDLWGRGTLVTVSS

SEQ ID NO: 209 - protein, 107 aa, synthetic construct
  REGION 1..107: note = Description of Artificial Sequence: Synthetic polypeptide
DIQMTQSPSSVSASVGDRVTITCRASQGISSWLAWYQQKPGKAPKLLIYAASNLQSGVPSRFSGSGSGTDFTLTISSLQPEDFATYYCQQGASFPITFGGGTKVEIK

SEQ ID NO: 210 - protein, 5 aa, synthetic construct
  REGION 1..5: note = Description of Artificial Sequence: Synthetic peptide
SYWMN

SEQ ID NO: 211 - protein, 17 aa, synthetic construct
  REGION 1..17: note = Description of Artificial Sequence: Synthetic peptide
MIDPSDSETHNAQKFQG

SEQ ID NO: 212 - protein, 8 aa, synthetic construct
  REGION 1..8: note = Description of Artificial Sequence: Synthetic peptide
LYRWYFDV

SEQ ID NO: 213 - protein, 11 aa, synthetic construct
  REGION 1..11: note = Description of Artificial Sequence: Synthetic peptide
RASEIVGTYVS

SEQ ID NO: 214 - protein, 7 aa, synthetic construct
  REGION 1..7: note = Description of Artificial Sequence: Synthetic peptide
GASNRYT

SEQ ID NO: 215 - protein, 9 aa, synthetic construct
  REGION 1..9: note = Description of Artificial Sequence: Synthetic peptide
GQSYNFPYT

SEQ ID NO: 216 - protein, 5 aa, synthetic construct
  REGION 1..5: note = Description of Artificial Sequence: Synthetic peptide
SYYMH

SEQ ID NO: 217 - protein, 17 aa, synthetic construct
  REGION 1..17: note = Description of Artificial Sequence: Synthetic peptide
IINPSGGSTSYAQKFQG

SEQ ID NO: 218 - protein, 10 aa, synthetic construct
  REGION 1..10: note = Description of Artificial Sequence: Synthetic peptide
STLWFSEFDY

SEQ ID NO: 219 - protein, 14 aa, synthetic construct
  REGION 1..14: note = Description of Artificial Sequence: Synthetic peptide
SGTSSDVGGHNYVS

SEQ ID NO: 220 - protein, 7 aa, synthetic construct
  REGION 1..7: note = Description of Artificial Sequence: Synthetic peptide
DVTKRPS

SEQ ID NO: 221 - protein, 9 aa, synthetic construct
  REGION 1..9: note = Description of Artificial Sequence: Synthetic peptide
LSYAGSRVY

SEQ ID NO: 222 - protein, 8 aa, synthetic construct
  REGION 1..8: note = Description of Artificial Sequence: Synthetic peptide
GYTFTSYW

SEQ ID NO: 223 - protein, 8 aa, synthetic construct
  REGION 1..8: note = Description of Artificial Sequence: Synthetic peptide
IDPSDSET

SEQ ID NO: 224 - protein, 10 aa, synthetic construct
  REGION 1..10: note = Description of Artificial Sequence: Synthetic peptide
ARLYRWYFDV

SEQ ID NO: 225 - protein, 6 aa, synthetic construct
  REGION 1..6: note = Description of Artificial Sequence: Synthetic peptide
EIVGTY

SEQ ID NO: 226 - skipped

SEQ ID NO: 227 - protein, 8 aa, synthetic construct
  REGION 1..8: note = Description of Artificial Sequence: Synthetic peptide
GYTFTSYY

SEQ ID NO: 228 - protein, 8 aa, synthetic construct
  REGION 1..8: note = Description of Artificial Sequence: Synthetic peptide
INPSGGST

SEQ ID NO: 229 - protein, 12 aa, synthetic construct
  REGION 1..12: note = Description of Artificial Sequence: Synthetic peptide
ARSTLWFSEFDY

SEQ ID NO: 230 - protein, 9 aa, synthetic construct
  REGION 1..9: note = Description of Artificial Sequence: Synthetic peptide
SSDVGGHNY

SEQ ID NO: 231 - skipped

SEQ ID NO: 232 - protein, 7 aa, synthetic construct
  REGION 1..7: note = Description of Artificial Sequence: Synthetic peptide
GYTFTSY

SEQ ID NO: 233 - protein, 4 aa, synthetic construct
  REGION 1..4: note = Description of Artificial Sequence: Synthetic peptide
PSDS

SEQ ID NO: 234 - protein, 6 aa, synthetic construct
  REGION 1..6: note = Description of Artificial Sequence: Synthetic peptide
YRWYFD

SEQ ID NO: 235 - protein, 7 aa, synthetic construct
  REGION 1..7: note = Description of Artificial Sequence: Synthetic peptide
SEIVGTY

SEQ ID NO: 236 - protein, 6 aa, synthetic construct
  REGION 1..6: note = Description of Artificial Sequence: Synthetic peptide
SYNFPY

SEQ ID NO: 237 - protein, 4 aa, synthetic construct
  REGION 1..4: note = Description of Artificial Sequence: Synthetic peptide
PSGG

SEQ ID NO: 238 - protein, 8 aa, synthetic construct
  REGION 1..8: note = Description of Artificial Sequence: Synthetic peptide
TLWFSEFD

SEQ ID NO: 239 - protein, 11 aa, synthetic construct
  REGION 1..11: note = Description of Artificial Sequence: Synthetic peptide
GTSSDVGGHNY

SEQ ID NO: 240 - protein, 6 aa, synthetic construct
  REGION 1..6: note = Description of Artificial Sequence: Synthetic peptide
YAGSRV

SEQ ID NO: 241 - protein, 10 aa, synthetic construct
  REGION 1..10: note = Description of Artificial Sequence: Synthetic peptide
ASGYTFTSYW

SEQ ID NO: 242 - protein, 17 aa, synthetic construct
  REGION 1..17: note = Description of Artificial Sequence: Synthetic peptide
IDPSDSETHNAQKFQGK

SEQ ID NO: 243 - protein, 7 aa, synthetic construct
  REGION 1..7: note = Description of Artificial Sequence: Synthetic peptide
LYRWYFD

SEQ ID NO: 244 - protein, 8 aa, synthetic construct
  REGION 1..8: note = Description of Artificial Sequence: Synthetic peptide
ASEIVGTY

SEQ ID NO: 245 - protein, 12 aa, synthetic construct
  REGION 1..12: note = Description of Artificial Sequence: Synthetic peptide
GASNRYTGVPAR

SEQ ID NO: 246 - protein, 6 aa, synthetic construct
  REGION 1..6: note = Description of Artificial Sequence: Synthetic peptide
SYNFPY

SEQ ID NO: 247 - protein, 10 aa, synthetic construct
  REGION 1..10: note = Description of Artificial Sequence: Synthetic peptide
ASGYTFTSYY

SEQ ID NO: 248 - protein, 17 aa, synthetic construct
  REGION 1..17: note = Description of Artificial Sequence: Synthetic peptide
INPSGGSTSYAQKFQGR

SEQ ID NO: 249 - protein, 9 aa, synthetic construct
  REGION 1..9: note = Description of Artificial Sequence: Synthetic peptide
STLWFSEFD

SEQ ID NO: 250 - protein, 12 aa, synthetic construct
  REGION 1..12: note = Description of Artificial Sequence: Synthetic peptide
DVTKRPSGVPDR

SEQ ID NO: 251 - protein, 7 aa, synthetic construct
  REGION 1..7: note = Description of Artificial Sequence: Synthetic peptide
YAGSRVY

SEQ ID NO: 252 - protein, 117 aa, synthetic construct
  REGION 1..117: note = Description of Artificial Sequence: Synthetic polypeptide
QVQLVQSGAEVVKPGASVKLSCKASGYTFTSYWMNWVRQRPGQGLEWIGMIDPSDSETHNAQKFQGKATLTVDKSTSTAYMHLSSLRSEDTAVYYCARLYRWYFDVWGAGTTVTVSS

SEQ ID NO: 253 - protein, 107 aa, synthetic construct
  REGION 1..107: note = Description of Artificial Sequence: Synthetic polypeptide
NIVMTQSPATMSMSPGERVTLSCRASEIVGTYVSWFQQKPGQAPRLLIYGASNRYTGVPARFSGSGSGTDFTLTISSVQPEDLADYHCGQSYNFPYTFGGGTKLEIK

SEQ ID NO: 254 - protein, 119 aa, synthetic construct
  REGION 1..119: note = Description of Artificial Sequence: Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARSTLWFSEFDYWGQGTLVTVSS

SEQ ID NO: 255 - protein, 110 aa, synthetic construct
  REGION 1..110: note = Description of Artificial Sequence: Synthetic polypeptide
QSVLTQPSSVSASPGQSITISCSGTSSDVGGHNYVSWYQQHPGKAPKLMIYDVTKRPSGVPDRFSGSKSGNTASLTVSGLQAEDEADYYCLSYAGSRVYVFGTGTKLTVL

SEQ ID NO: 256 - protein, 15 aa, synthetic construct
  REGION 1..15: note = Description of Artificial Sequence: Synthetic peptide
RRCPLYISYDPVCRR

SEQ ID NO: 257 - protein, 19 aa, synthetic construct
  REGION 1..19: note = Description of Artificial Sequence: Synthetic peptide
RRRRCPLYISYDPVCRRRR

SEQ ID NO: 258 - protein, 5 aa, synthetic construct
  REGION 1..5: note = Description of Artificial Sequence: Synthetic polypeptide
NYGMN

SEQ ID NO: 259 - protein, 17 aa, synthetic construct
  REGION 1..17: note = Description of Artificial Sequence: Synthetic polypeptide
WINTYTGEPTYAADFKR

SEQ ID NO: 260 - protein, 14 aa, synthetic construct
  REGION 1..14: note = Description of Artificial Sequence: Synthetic polypeptide
YPHYYGSSHWYFDV

SEQ ID NO: 261 - protein, 11 aa, synthetic construct
  REGION 1..11: note = Description of Artificial Sequence: Synthetic polypeptide
SASQDISNYLN

SEQ ID NO: 262 - protein, 7 aa, synthetic construct
  REGION 1..7: note = Description of Artificial Sequence: Synthetic polypeptide
FTSSLHS

SEQ ID NO: 263 - protein, 9 aa, synthetic construct
  REGION 1..9: note = Description of Artificial Sequence: Synthetic polypeptide
QQYSTVPWT

SEQ ID NO: 264 - protein, 5 aa, synthetic construct
  REGION 1..5: note = Description of Artificial Sequence: Synthetic polypeptide
HYGMN

SEQ ID NO: 265 - protein, 14 aa, synthetic construct
  REGION 1..14: note = Description of Artificial Sequence: Synthetic polypeptide
YPYYYGTSHWYFDV

SEQ ID NO: 266 - protein, 6 aa, synthetic construct
  REGION 1..6: note = Description of Artificial Sequence: Synthetic polypeptide
NNDVMC

SEQ ID NO: 267 - protein, 17 aa, synthetic construct
  REGION 1..17: note = Description of Artificial Sequence: Synthetic polypeptide
CIMTTDVVTEYANWAKS

SEQ ID NO: 268 - protein, 12 aa, synthetic construct
  REGION 1..12: note = Description of Artificial Sequence: Synthetic polypeptide
DSVGSPLMSFDL

SEQ ID NO: 269 - protein, 13 aa, synthetic construct
  REGION 1..13: note = Description of Artificial Sequence: Synthetic polypeptide
QASQSIYNNNELS

SEQ ID NO: 270 - protein, 7 aa, synthetic construct
  REGION 1..7: note = Description of Artificial Sequence: Synthetic polypeptide
RASTLAS

SEQ ID NO: 271 - protein, 12 aa, synthetic construct
  REGION 1..12: note = Description of Artificial Sequence: Synthetic polypeptide
GGYKSYSNDGNG

SEQ ID NO: 272 - protein, 6 aa, synthetic construct
  REGION 1..6: note = Description of Artificial Sequence: Synthetic polypeptide
DYYYMT

SEQ ID NO: 273 - protein, 16 aa, synthetic construct
  REGION 1..16: note = Description of Artificial Sequence: Synthetic polypeptide
FIDPDDDPYYATWAKG

SEQ ID NO: 274 - protein, 11 aa, synthetic construct
  REGION 1..11: note = Description of Artificial Sequence: Synthetic polypeptide
GDHNSGWGLDI

SEQ ID NO: 275 - protein, 11 aa, synthetic construct
  REGION 1..11: note = Description of Artificial Sequence: Synthetic polypeptide
QASEIIHSWLA

SEQ ID NO: 276 - protein, 7 aa, synthetic construct
  REGION 1..7: note = Description of Artificial Sequence: Synthetic polypeptide
LASTLAS

SEQ ID NO: 277 - protein, 12 aa, synthetic construct
  REGION 1..12: note = Description of Artificial Sequence: Synthetic polypeptide
QNVYLASTNGAN

SEQ ID NO: 278 - protein, 8 aa, synthetic construct
  REGION 1..8: note = Description of Artificial Sequence: Synthetic polypeptide
GYTFTNYG

SEQ ID NO: 279 - protein, 8 aa, synthetic construct
  REGION 1..8: note = Description of Artificial Sequence: Synthetic polypeptide
INTYTGEP

SEQ ID NO: 280 - protein, 16 aa, synthetic construct
  REGION 1..16: note = Description of Artificial Sequence: Synthetic polypeptide
AKYPHYYGSSHWYFDV

SEQ ID NO: 281 - protein, 6 aa, synthetic construct
  REGION 1..6: note = Description of Artificial Sequence: Synthetic polypeptide
QDISNY

SEQ ID NO: 282 - skipped

SEQ ID NO: 283 - protein, 8 aa, synthetic construct
  REGION 1..8: note = Description of Artificial Sequence: Synthetic polypeptide
GYDFTHYG

SEQ ID NO: 284 - protein, 16 aa, synthetic construct
  REGION 1..16: note = Description of Artificial Sequence: Synthetic polypeptide
AKYPYYYGTSHWYFDV

SEQ ID NO: 285 - protein, 9 aa, synthetic construct
  REGION 1..9: note = Description of Artificial Sequence: Synthetic polypeptide
GFSFSNNDV

SEQ ID NO: 286 - protein, 8 aa, synthetic construct
  REGION 1..8: note = Description of Artificial Sequence: Synthetic polypeptide
IMTTDVVT

SEQ ID NO: 287 - protein, 14 aa, synthetic construct
  REGION 1..14: note = Description of Artificial Sequence: Synthetic polypeptide
ARDSVGSPLMSFDL

SEQ ID NO: 288 - protein, 8 aa, synthetic construct
  REGION 1..8: note = Description of Artificial Sequence: Synthetic polypeptide
QSIYNNNE

SEQ ID NO: 289 - skipped

SEQ ID NO: 290 - protein, 9 aa, synthetic construct
  REGION 1..9: note = Description of Artificial Sequence: Synthetic polypeptide
GFSLTDYYY

SEQ ID NO: 291 - protein, 7 aa, synthetic construct
  REGION 1..7: note = Description of Artificial Sequence: Synthetic polypeptide
IDPDDDP

SEQ ID NO: 292 - protein, 13 aa, synthetic construct
  REGION 1..13: note = Description of Artificial Sequence: Synthetic polypeptide
AGGDHNSGWGLDI

SEQ ID NO: 293 - protein, 6 aa, synthetic construct
  REGION 1..6: note = Description of Artificial Sequence: Synthetic polypeptide
EIIHSW

SEQ ID NO: 294 - skipped

SEQ ID NO: 295 - protein, 7 aa, synthetic construct
  REGION 1..7: note = Description of Artificial Sequence: Synthetic polypeptide
GYTFTNY

SEQ ID NO: 296 - protein, 4 aa, synthetic construct
  REGION 1..4: note = Description of Artificial Sequence: Synthetic polypeptide
TYTG

SEQ ID NO: 297 - protein, 12 aa, synthetic construct
  REGION 1..12: note = Description of Artificial Sequence: Synthetic polypeptide
PHYYGSSHWYFD

SEQ ID NO: 298 - protein, 7 aa, synthetic construct
  REGION 1..7: note = Description of Artificial Sequence: Synthetic polypeptide
SQDISNY

SEQ ID NO: 299 - protein, 6 aa, synthetic construct
  REGION 1..6: note = Description of Artificial Sequence: Synthetic polypeptide
YSTVPW

SEQ ID NO: 300 - protein, 7 aa, synthetic construct
  REGION 1..7: note = Description of Artificial Sequence: Synthetic polypeptide
GYDFTHY

SEQ ID NO: 301 - protein, 12 aa, synthetic construct
  REGION 1..12: note = Description of Artificial Sequence: Synthetic polypeptide
PYYYGTSHWYFD

SEQ ID NO: 302 - protein, 8 aa, synthetic construct
  REGION 1..8: note = Description of Artificial Sequence: Synthetic polypeptide
GFSFSNND

SEQ ID NO: 303 - protein, 4 aa, synthetic construct
  REGION 1..4: note = Description of Artificial Sequence: Synthetic polypeptide
TTDV

SEQ ID NO: 304 - protein, 10 aa, synthetic construct
  REGION 1..10: note = Description of Artificial Sequence: Synthetic polypeptide
SVGSPLMSFD

SEQ ID NO: 305 - protein, 9 aa, synthetic construct
  REGION 1..9: note = Description of Artificial Sequence: Synthetic polypeptide
SQSIYNNNE

SEQ ID NO: 306 - protein, 9 aa, synthetic construct
  REGION 1..9: note = Description of Artificial Sequence: Synthetic polypeptide
YKSYSNDGN

SEQ ID NO: 307 - protein, 8 aa, synthetic construct
  REGION 1..8: note = Description of Artificial Sequence: Synthetic polypeptide
GFSLTDYY

SEQ ID NO: 308 - skipped

SEQ ID NO: 309 - protein, 9 aa, synthetic construct
  REGION 1..9: note = Description of Artificial Sequence: Synthetic polypeptide
DHNSGWGLD

SEQ ID NO: 310 - protein, 7 aa, synthetic construct
  REGION 1..7: note = Description of Artificial Sequence: Synthetic polypeptide
SEIIHSW

SEQ ID NO: 311 - protein, 9 aa, synthetic construct
  REGION 1..9: note = Description of Artificial Sequence: Synthetic polypeptide
VYLASTNGA

SEQ ID NO: 312 - protein, 10 aa, synthetic construct
  REGION 1..10: note = Description of Artificial Sequence: Synthetic polypeptide
ASGYTFTNYG

SEQ ID NO: 313 - protein, 17 aa, synthetic construct
  REGION 1..17: note = Description of Artificial Sequence: Synthetic polypeptide
INTYTGEPTYAADFKRR

SEQ ID NO: 314 - protein, 13 aa, synthetic construct
  REGION 1..13: note = Description of Artificial Sequence: Synthetic polypeptide
YPHYYGSSHWYFD

SEQ ID NO: 315 - protein, 8 aa, synthetic construct
  REGION 1..8: note = Description of Artificial Sequence: Synthetic polypeptide
ASQDISNY

SEQ ID NO: 316 - protein, 12 aa, synthetic construct
  REGION 1..12: note = Description of Artificial Sequence: Synthetic polypeptide
FTSSLHSGVPSR

SEQ ID NO: 317 - protein, 10 aa, synthetic construct
  REGION 1..10: note = Description of Artificial Sequence: Synthetic polypeptide
ASGYDFTHYG

SEQ ID NO: 318 - protein, 13 aa, synthetic construct
  REGION 1..13: note = Description of Artificial Sequence: Synthetic polypeptide
YPYYYGTSHWYFD

SEQ ID NO: 319 - protein, 11 aa, synthetic construct
  REGION 1..11: note = Description of Artificial Sequence: Synthetic polypeptide
ASGFSFSNNDV

SEQ ID NO: 320 - protein, 17 aa, synthetic construct
  REGION 1..17: note = Description of Artificial Sequence: Synthetic polypeptide
IMTTDVVTEYANWAKSR

SEQ ID NO: 321 - protein, 11 aa, synthetic construct
  REGION 1..11: note = Description of Artificial Sequence: Synthetic polypeptide
DSVGSPLMSFD

SEQ ID NO: 322 - protein, 10 aa, synthetic construct
  REGION 1..10: note = Description of Artificial Sequence: Synthetic polypeptide
ASQSIYNNNE

SEQ ID NO: 323 - protein, 12 aa, synthetic construct
  REGION 1..12: note = Description of Artificial Sequence: Synthetic polypeptide
RASTLASGVPSR

SEQ ID NO: 324 - protein, 11 aa, synthetic construct
  REGION 1..11: note = Description of Artificial Sequence: Synthetic polypeptide
ASGFSLTDYYY

SEQ ID NO: 325 - protein, 16 aa, synthetic construct
  REGION 1..16: note = Description of Artificial Sequence: Synthetic polypeptide
IDPDDDPYYATWAKGR

SEQ ID NO: 326 - protein, 10 aa, synthetic construct
  REGION 1..10: note = Description of Artificial Sequence: Synthetic polypeptide
GDHNSGWGLD

SEQ ID NO: 327 - protein, 8 aa, synthetic construct
  REGION 1..8: note = Description of Artificial Sequence: Synthetic polypeptide
ASEIIHSW

SEQ ID NO: 328 - protein, 12 aa, synthetic construct
  REGION 1..12: note = Description of Artificial Sequence: Synthetic polypeptide
LASTLASGVPSR

SEQ ID NO: 329 - protein, 123 aa, synthetic construct [sequence omitted]

SEQ ID NO: 330 - protein, 108 aa, synthetic construct
  REGION 1..108: note = Description of Artificial Sequence: Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 331 - protein, 123 aa, synthetic construct [sequence omitted]

SEQ ID NO: 332 - protein, 108 aa, synthetic construct
  REGION 1..108: note = Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 333 - protein, 122 aa, synthetic construct
  REGION 1..122: note = Description of Artificial Sequence: Synthetic polypeptide
EVQLVESGGGLVQPGGSLRLSCAASGYDFTHYGMNVRQAPGKGLEWVGWINTYTGEPTYAADFKRRFTFSLDTSKSTAYLQMNSLRAEDTAVYYCAKYPYYYGTSHWYFDVWGQGTLYTVSS

SEQ ID NO: 334 - protein, 108 aa, synthetic construct; identical to SEQ ID NO: 332
  REGION 1..108: note = Description of Artificial Sequence: Synthetic polypeptide

SEQ ID NO: 335 - protein, 122 aa, synthetic construct
  REGION 1..122: note = Description of Artificial Sequence: Synthetic polypeptide
EVQLVESGGGLVKPGGSLRLSCAASGFSFSNNDVMCWVRQAPGKGLEWIGCIMTTDVVTEYANWAKSRFTVSRDSAKNSVYLQMNSLRAEDTAVYFCARDSVGSPLMSFDLWGPGTLVTVSS

SEQ ID NO: 336 - protein, 112 aa, synthetic construct
  REGION 1..112: note = Description of Artificial Sequence: Synthetic polypeptide
DIQMTQSPSSLSASVGDRVTINCQASQSIYNNNELSWYQQKPGKPPKLLIYRASTLASGVPSRFSGSGSGTDFTLTISSLQPEDVATYYCGGYKSYSNDGNGFGGGTKVEIK

SEQ ID NO: 337 - protein, 120 aa, synthetic construct
  REGION 1..120: note = Description of Artificial Sequence: Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 338 - protein, 110 aa, synthetic construct
  REGION 1..110: note = Description of Artificial Sequence: Synthetic polypeptide
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVL

SEQ ID NO: 339 - protein, 5 aa, synthetic construct
HYYWS

SEQ ID NO: 340 - protein, 8 aa, synthetic construct
GGSIEHYY

SEQ ID NO: 341 - protein, 7 aa, synthetic construct
GGSIEHY

SEQ ID NO: 342 - protein, 10 aa, synthetic construct
VSGGSIEHYY

SEQ ID NO: 343 - protein, 115 aa, synthetic construct
QVQLQESGPGLVKPSETLSLTCTVSGGSIEHYYWSWIRQPPGKGLEWIGYIYYSGSTNYNPSLKSRVTISVDTSKNQFSLKLSSVTAADTAVYYCARGKTGSAAWGQGTLVTVSS

SEQ ID NO: 344 - protein, 107 aa, synthetic construct; identical to SEQ ID NO: 92

SEQ ID NO: 345 - protein, 5 aa, synthetic construct
SYAMS

SEQ ID NO: 346 - protein, 17 aa, synthetic construct
AISGSGGSTYYADSVKG

SEQ ID NO: 347 - protein, 7 aa, synthetic construct
SYGAFDY

SEQ ID NO: 348 - protein, 11 aa, synthetic construct
RASQSISSYLN

SEQ ID NO: 349 - protein, 10 aa, synthetic construct
QQMHPRAPKT

SEQ ID NO: 350 - protein, 8 aa, synthetic construct
GFTFSSYA

SEQ ID NO: 351 - protein, 8 aa, synthetic construct
ISGSGGST

SEQ ID NO: 352 - protein, 9 aa, synthetic construct
AKSYGAFDY

SEQ ID NO: 353 - protein, 6 aa, synthetic construct
QSISSY

SEQ ID NO: 354 - protein, 10 aa, synthetic construct
QQMHPRAPKT

SEQ ID NO: 355 - protein, 7 aa, synthetic construct
GFTFSSY

SEQ ID NO: 356 - protein, 4 aa, synthetic construct
GSGG

SEQ ID NO: 357 - protein, 5 aa, synthetic construct
YGAFD

SEQ ID NO: 358 - protein, 7 aa, synthetic construct
SQSISSY

SEQ ID NO: 359 - protein, 7 aa, synthetic construct
MHPRAPK

SEQ ID NO: 360 - protein, 10 aa, synthetic construct
ASGFTFSSYA

SEQ ID NO: 361 - protein, 17 aa, synthetic construct
ISGSGGSTYYADSVKGR

SEQ ID NO: 362 - protein, 6 aa, synthetic construct
SYGAFD

SEQ ID NO: 363 - protein, 8 aa, synthetic construct
ASQSISSY

SEQ ID NO: 364 - protein, 115 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLTVSS

SEQ ID NO: 365 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQMHPRAPKTFGQGTKVEIK

(19) United States
(12) Patent Application Publication
(10) Pub. No.: US 2023/0365682 A1
(43) Pub. Date: Nov. 16, 2023

(54) COMBINATION THERAPY FOR TREATING COLORECTAL CANCER

(71) Applicant: Gilead Sciences, Inc., Foster City, CA (US)

(72) Inventors: Mark P. Chao, Palo Alto, CA (US); William J. Grossman, Danville, CA (US); Fatema A. Legrand, Palo Alto, CA (US)

(21) Appl. No.: 18/295,163

(22) Filed: Apr. 3, 2023

Related U.S. Application Data
(60) Provisional application No. 63/327,584, filed on Apr. 5, 2022.

(51) Int. Cl. C07K 16/28; C07K 16/22; A61P 35/04; A61P 1/00; A61P 35/00; A61K 31/513; A61K 31/4745; A61K 31/506; A61K 31/4709; A61K 31/44; A61K 31/4439; A61K 31/47; A61K 31/404; A61K 31/7072; C07K 14/71; C07K 14/705; A61K 31/7068; A61K 31/282; A61K 31/519

(52) U.S. Cl. CPC C07K 16/2803; C07K 16/2896; C07K 16/22; C07K 16/2863; C07K 16/2818; C07K 16/2827; A61P 35/04; A61P 1/00; A61P 35/00; A61K 31/513; A61K 31/4745; C07K 16/2851; C07K 16/2866; C07K 16/2878; A61K 31/506; A61K 31/4709; A61K 31/44; A61K 31/4439; A61K 31/47; A61K 31/404; A61K 31/7072; C07K 14/71; C07K 14/70596; A61K 31/7068; A61K 31/282; A61K 31/519; C07K 2319/30; A61K 2039/507

(57) ABSTRACT

Provided are methods of treating colorectal cancer in a subject comprising co-administering to the subject an effective amount of: (a) an agent that inhibits binding between CD47 and SIRPα (e.g., magrolimab); and (b) an agent that inhibits binding between vascular endothelial growth factor A (VEGFA) and one or more VEGFA cognate receptors (e.g., bevacizumab), optionally further including a chemotherapy regimen (e.g., FOLFIRI).

### CROSS-REFERENCE TO RELATED APPLICATIONS

This application claims the benefit under 35 U.S.C. § 119(e) of U.S. Provisional Application No. 63/327,584, filed on Apr. 5, 2022, which is hereby incorporated herein by reference in its entirety for all purposes.

### SEQUENCE LISTING

The instant application contains a Sequence Listing which has been submitted electronically in .XML file format and is hereby incorporated by reference in its entirety. Said .XML copy, created on created on Jan. 14, 2023, is named 1433-WO-PCT_SL.xml and is 444,843 bytes in size.

### BACKGROUND

Metastatic colorectal adenocarcinoma ranks as the second most lethal cancer and the third most prevalent malignant tumor worldwide. In 2018, 1.8 million new cases and 881,000 deaths were reported, which accounted for nearly 10% of new cancer cases and deaths worldwide (Bray, et al., CA Cancer J Clin (2018) 68(6):394-424). The number of new cases is expected to increase to nearly 2.5 million in 2035 (Dekker, et al., Lancet (2019) 394 (10207):1467-80). In the United States (US), metastatic colorectal cancer (mCRC) is the second leading cause of cancer deaths (Siegel, et al., CA Cancer J Clin (2018) 68 (1):7-30). In 2020, 148,000 new diagnoses and 53,200 deaths were reported, including 17,930 cases and 3,640 deaths in individuals younger than 50 years of age. The 5-year survival rate is approximately 15% (Surveillance Epidemiology and End Results (SEER) 2021; https://seer.cancer.gov). Although screening strategies and new treatment modalities have started to reduce the overall CRC death rate, the development of advanced metastatic disease is still associated with poor long-term survival. Recent data has demonstrated a continued decline in incidence and mortality in patients at least 65 years of age but a converse increase among those less than 65 years of age. The etiology of this phenomenon has yet to be determined and differences in genetics may be a factor.

Colorectal cancer is a heterogeneous disease complicated by the common occurrence of several molecular alterations comprising the epidermal growth factor receptor (EGFR) pathway, including mutations in Kirsten rat sarcoma (KRAS), neuroblastoma RAS viral oncogene homolog (NRAS), and v-raf murine sarcoma viral oncogene homolog B1 (BRAF [V600E]), and in the human epidermal growth factor receptor 2 (HER2) and MET receptors. Other molecular alterations include DNA damage repair mechanisms and rare kinase fusions. Moreover, tumor sidedness is associated with distinct clinical and biological characteristics. Right-sided CRC is more common in women, and associated with Lynch syndrome, mitogen-activated protein kinase (MAPK)-signaling, high microsatellite instability (MSI-H), deficiency of mismatch repair genes, CpG island methylation, and KRAS and BRAF mutations. Left-sided CRC is more common in men, and associated with familial adenomatous polyposis syndrome, wingless-related integration site (Wnt) and EGFR signaling, chromosomal instability, v-erb-b2 erythroblastic leukemia viral oncogene homolog 1 (ERBB1) and ERBB2 amplifications, adenomatous polyposis coli (APC), p53, and NRAS mutations. These alterations represent oncogenic drivers that may coexist in the same tumor with other primary and acquired alterations via a clonal selection process.

Advances have been made in the therapy of CRC in targeted subgroups with specific mutational profiles. However, resistance to these targeted agents as well as standard chemotherapies based on specific molecular alterations have confounded treatment. Increasingly, enhanced knowledge about tumor biology is driving therapeutic decision-making. Optimal combinations and sequencing of these agents is continuing to evolve. Known biologic drugs that are active against mCRC include agents targeting vascular endothelial growth factors (VEGFs) and their receptors (VEGFRs), EGFR, BRAF V600E, HER2, immunotherapy using immune checkpoint inhibitors, and tropomyosin receptor kinase (TRK) inhibitors. Biomarkers have been defined for patients who are candidates for agents targeting EGFR, HER2, TRK fusions, and for immunotherapy, but are not yet defined for other agents.

Systemic chemotherapy in combination with anti EGFR antibodies such as cetuximab and panitumumab have significantly improved prognosis in KRAS wild-type patients (Lee, et al., Clin Colorectal Cancer (2015) 14(4):203-18). In addition, patients with MSI-H have demonstrated impressive responses to checkpoint inhibitors such as the anti-programmed cell death-1 (PD-1) agent pembrolizumab (Le, et al., N Engl J Med (2015) 372 (26):2509-20; Overman, et al., Abstract 3501, American Society of Clinical Oncology (ASCO) Annual Meeting; 2016 03-07 June; Chicago, Illinois), but this represents only a subset of this tumor type (10% to 15% at diagnosis). On the other hand, certain subsets of patients such as those with KRAS mutations, which comprise over 40% of CRCs (Vaughn, et al., Genes Chromosomes Cancer (2011) 50(5):307-12), do not respond to anti-EGFR antibody therapies (Allegra, et al., J Clin Oncol (2009) 27(12):2091-6). Patients with KRAS mutant mCRC have a poorer prognosis while having limited available therapies. For the majority of patients who are ineligible or unable to derive benefit from novel targeted therapies or immunotherapies, standard of care (SOC) doublet or triplet chemotherapy based regimens that are associated with systemic toxicity, unsatisfying response rates, unpredictable innate and acquired resistance, as well as low tumor-specific selectivity are the only available treatment options.

### SUMMARY

In one aspect, provided is a method of treating previously treated advanced inoperable metastatic colorectal cancer in a subject comprising co-administering to the subject an effective amount of: (a) an agent that inhibits binding between CD47 and SIRPα; and (b) an agent that inhibits binding between vascular endothelial growth factor A (VEGFA) and one or more VEGFA cognate receptors. In some embodiments, the one or more VEGFA cognate receptors are selected from fms related receptor tyrosine kinase 1 (FLT1, a.k.a., VEGFR1) and kinase insert domain receptor (KDR, a.k.a., VEGFR2). In some embodiments, the agent that inhibits binding between CD47 and SIRPα comprises an antibody that binds to CD47. In some embodiments, the antibody that binds to CD47 is selected from magrolimab, lemzoparlimab, letaplimab, ligufalimab, AO-176, simridarlimab (IBI-322), gentulizumab, ZL-1201, IMC-002, SRF-231, CC-90002 (a.k.a., INBRX-103), NI-1701 (a.k.a., TG-1801) and STI-6643. In some embodiments, the agent that inhibits binding between CD47 and SIRPα comprises an antibody that binds to SIRPα. In some embodiments, the antibody that binds to SIRPα is selected from GS-0189 (a.k.a., FSI-189), CC-95251, BI-765063 and APX-700. In some embodiments, the agent that inhibits binding between CD47 and SIRPα comprises a SIRPα-Fc fusion protein. In some embodiments, the SIRPα-Fc fusion protein is selected from evorpacept (ALX-148), timdarpacept, TTI-621, TTI-622, JMT601 (CPO107) and SL-172154. In some embodiments, the agent that inhibits binding between VEGFA and one or more VEGFA cognate receptors comprises an antibody that binds to VEGFA. In some embodiments, the antibody that binds to VEGFA is selected from bevacizumab, ranibizumab, brolucizumab, ivonescimab, sevacizumab and faricimab. In some embodiments, the agent that inhibits binding between VEGFA and one or more VEGFA cognate receptors comprises an antibody that binds to VEGFR2. In some embodiments, the antibody that binds to VEGFR2 is ramucirumab. In some embodiments, the agent that inhibits binding between VEGFA and one or more VEGFA cognate receptors comprises a VEGFA-Fc fusion protein. In some embodiments, the VEGFA-Fc fusion protein is selected from conbercept and aflibercept. In some embodiments, the agent that inhibits binding between VEGFA and one or more VEGFA cognate receptors comprises a small molecule inhibitor. In some embodiments, the small molecule is selected from surufatinib, catequentinib hydrochloride, fruquintinib, tivozanib, regorafenib, axitinib, vandetanib, chiauranib, pazopanib hydrochloride, sunitinib malate and pegaptanib octasodium.

In another aspect, provided is a method of treating, mitigating, reducing, preventing or delaying the recurrence or metastasis of, colorectal cancer (CRC) in a subject comprising co-administering to the subject an effective amount of magrolimab and bevancizumab. In some embodiments, the cancer is (i) unresectable, locally advanced or (ii) metastatic. In some embodiments, the cancer is unresectable, locally advanced and the subject is treatment naïve. In another aspect, provided is a method of treating previously treated advanced inoperable metastatic colorectal cancer in a subject comprising co-administering to the subject an effective amount of: (a) magrolimab; and (b) bevancizumab. In some embodiments, the magrolimab is first administered at a priming dose of less than 10 mg/kg and then administered at one or more therapeutic doses of at least 15 mg/kg, e.g., at least 30 mg/kg, 45 mg/kg, 60 mg/kg. In some embodiments, the magrolimab is first administered at a priming dose of less than 5 mg/kg and then administered at one or more therapeutic doses of at least 30 mg/kg, e.g., 45 mg/kg, 60 mg/kg. In some embodiments, the magrolimab is first administered at a priming dose of 1 mg/kg, then administered at one or more therapeutic doses of 30 mg/kg, followed by administration of one or more therapeutic doses of 60 mg/kg. In some embodiments, the magrolimab is first administered at a priming dose of 1 mg/kg, then administered at one or more therapeutic doses of 20 mg/kg, followed by administration of one or more therapeutic doses of 45 mg/kg. In some embodiments, the magrolimab is first administered at a priming dose of 1 mg/kg, then administered at one or more therapeutic doses of 15 mg/kg, followed by administration of one or more therapeutic doses of 30 mg/kg. In some embodiments, the magrolimab is administered intravenously, subcutaneously or intratumorally. In some embodiments, the bevacizumab is administered at one or more doses in the range of 5 mg/kg to 15 mg/kg, e.g., 5 mg/kg to 7.5 mg/kg or 7.5 mg/kg to 15 mg/kg. In some embodiments, the bevacizumab is administered at one or more doses of 5 mg/kg. In some embodiments, the bevacizumab is administered intravenously, subcutaneously or intratumorally. In some embodiments, the magrolimab and the bevacizumab are administered for first, second and third 28-day cycles, wherein: (a) for the first 28-day cycle, magrolimab is administered at a dose of 1 mg/kg on day 1 and at a dose of 30 mg/kg weekly (QW) beginning on day 8; and bevacizumab is administered at a dose of 5 mg/kg on days 1 and 15; (b) for the second 28-day cycle, magrolimab is administered at a dose of 30 mg/kg weekly (QW); and bevacizumab is administered at a dose of 5 mg/kg on days 1 and 15; and (c) for the third 28-day cycle, magrolimab is administered at a dose of 30 mg/kg Q2W; and bevacizumab is administered at a dose of 5 mg/kg on days 1 and 15. In some embodiments, the magrolimab and the bevacizumab are administered for first, second and third 28-day cycles, wherein: (a) for the first 28-day cycle, magrolimab is administered at a dose of 1 mg/kg on day 1 and at a dose of 20 mg/kg weekly (QW) beginning on day 8; and bevacizumab is administered at a dose of 5 mg/kg on days 1 and 15; (b) for the second 28-day cycle, magrolimab is administered at a dose of 20 mg/kg weekly (QW); and bevacizumab is administered at a dose of 5 mg/kg on days 1 and 15; and (c) for the third 28-day cycle, magrolimab is administered at a dose of 20 mg/kg Q2W; and bevacizumab is administered at a dose of 5 mg/kg on days 1 and 15. In some embodiments, the magrolimab and the bevacizumab are administered for first, second and third 28-day cycles, wherein: (a) for the first 28-day cycle, magrolimab is administered at a dose of 1 mg/kg on day 1 and at a dose of 15 mg/kg weekly (QW) beginning on day 8; and bevacizumab is administered at a dose of 5 mg/kg on days 1 and 15; (b) for the second 28-day cycle, magrolimab is administered at a dose of 15 mg/kg weekly (QW); and bevacizumab is administered at a dose of 5 mg/kg on days 1 and 15; and (c) for the third 28-day cycle, magrolimab is administered at a dose of 15 mg/kg Q2W; and bevacizumab is administered at a dose of 5 mg/kg on days 1 and 15.

With respect to further embodiments of the methods set forth above and herein, in some embodiments, the method further comprises co-administering a chemotherapy regimen. In some embodiments, the method comprises co-administering a FOLFOX regimen, a FOLFIRI regimen, a XELIRI (a.k.a., CAPIRI) regimen, a FOLFOXIRI regimen, a XELOXIRI regimen or a FOLFIRINOX regimen. In some embodiments, the method comprises co-administering a FOLFIRI regimen or a XELIRI regimen. In some embodiments, the method comprises co-administering a FOLFIRI regimen. In some embodiments, the cancer has cell surface expression of CD47. In some embodiments, the cancer does not comprise a BRAF V600E mutation. In some embodiments, the cancer does not comprise high or observable or detectable microsatellite instability. In some embodiments, the cancer has one or more KRAS mutations and the subject has not responded (e.g., the cancer progressed or did not regress) to anti-EGFR antibody therapy. In some embodiments, the cancer is adenocarcinoma originating in the colon or rectum. In some embodiments, the cancer has progressed after one or more prior systemic therapies. In some embodiments, the one or more prior therapies comprise administration of one or more agents selected from the group consisting of 5-fluorouracil (5-FU), oxaliplatin, bevacizumab, cetuximab and panitumumab. In some embodiments, the treatment results in a reduction in overall tumor burden of at least 15%, at least 20%, at least 30%, or at least 40%, as determined using linear dimensional methods (e.g. RECIST v1.1). In some embodiments, the method comprises reducing in size or eliminating the metastases. In some embodiments, the method further comprises administering one or more therapeutic antibodies. In some embodiments, the method comprises co-administering an antibody that binds to epidermal growth factor receptor (EGFR). In some embodiments, the antibody that binds to EGFR is selected from cetuximab and panitumumab. In some embodiments, the method further comprises co-administering one or more blockers or inhibitors of one or more T-cell stimulatory immune checkpoint proteins or receptors. In some embodiments, the one or more immune checkpoint inhibitors comprises a proteinaceous (e.g., antibody) inhibitor of PD-L1 (CD274), PD-1 (PDCD1) or CTLA4. In some embodiments, the one or more immune checkpoint proteins or receptors are selected from: CD274 (CD274, PDL1, PD-L1) and programmed cell death 1 (PDCD1, PD1, PD-1). In some embodiments, the proteinaceous (e.g., antibody) inhibitor of CTLA4 is selected from ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In some embodiments, the proteinaceous (e.g., antibody) inhibitor of programmed cell death 1 (PDCD1; NCBI Gene ID: 5133; CD279, PD-1, PD1) is selected from zimberelimab (AB122, GLS-010, WBP-3055), pembrolizumab (KEYTRUDA®, MK-3475, SCH900475), nivolumab (OPDIVO®, BMS-936558, MDX-1106), cemiplimab (LIBTAYO®; cemiplimab-rwlc, REGN-2810), pidilizumab (CT-011), AMG-404, MEDI0680 (AMP-514), spartalizumab (PDR001), tislelizumab (BGB-A317), toripalimab (JS-001), genolimzumab (CBT-501, APL-501, GB 226), SHR-1201, camrelizumab (SHR-1210), sintilimab (TYVYT®; IBI-308), dostarlimab (TSR-042, WBP-285), lambrolizumab (MK-3475); sasanlimab (PF-06801591), cetrelimab (JNJ-63723283), serplulimab (HLX-10), retifanlimab (MGA-012), balstilimab (AGEN2034), prolgolimab (BCD 100), budigalimab (ABBV-181), vopratelimab (JTX-4014), AK-103 (HX-008), AK-105, CS 1003, BI-754091, LZM-009, Sym-021, BAT-1306, PD1-PIK, tebotelimab (MGD013; PD-1/LAG-3), RO-7247669 (PD-1/LAG-3), FS-118 (LAG-3/PD-L1), RO-7121661 (PD 1/TIM-3), RG7769 (PD-1/TIM-3), PF-06936308 (PD 1/CTLA4), MGD-019 (PD-1/CTLA4), KN-046 (PD 1/CTLA4), XmAb-20717 (PD 1/CTLA4), AK-104 (CTLA4/PD-1) and MEDI-5752 (CTLA4/PD-1). In some embodiments, the proteinaceous (e.g., antibody) inhibitor of CD274 molecule (NCBI Gene ID: Gene ID: 29126; B7-H, B7H1, PD-L1) is selected from atezolizumab (TECENTRIQ®), avelumab (BAVENCIO®; MSB0010718C), envafolimab (ASC22), durvalumab (IMFINZI®; MEDI-4736), BMS-936559 (MDX1105), cosibelimab (CK-301), lodapolimab (LY 3300054), garivulimab (BGB A333), envafolimab (KN035), opucolimab (HLX 20), manelimab (BCD 135), CX-072, CBT-502 (TQB2450), MSB-2311, SHR-1316, sugemalimab (CS-1001; WBP3155), A167 (KL-A167, HBM 9167), STI-A1015 (IMC-001), FAZ-053, BMS-936559 (MDX1105), INCB086550, GEN-1046 (PD-L1/4-1BB), FPT-155 (CTLA4/PD-L1/CD28), M7824 (PD-L1/TGFβ-EC domain), CA-170 (PD-L1/VISTA), CDX-527 (CD27/PD-L1), LY-3415244 (TIM-3/PDL1), INBRX-105 (4-1BB/PDL1) and GNS-1480 (PD-L1/EGFR). In some embodiments, the method comprises co-administering an agonist of fms related receptor tyrosine kinase 3 (FLT3). In some embodiments, the agonist of FLT3 is selected from GS-3583 and CDX-301. In some embodiments, the agent that inhibits binding between CD47 and SIRPα and the agent that inhibits binding between VEGFA and one or more VEGFA cognate receptors are administered in a combined synergistic amount. In some embodiments, the administration of the agent that inhibits binding between CD47 and SIRPα and the agent that inhibits binding between VEGFA and one or more VEGFA cognate receptors provides a synergistic effect. In some embodiments, the synergistic effect is increased cancer cell death and/or decreased cancer cell growth when comparing the effect of the combination versus either the agent that inhibits binding between CD47 and SIRPα or the agent that inhibits binding between VEGFA and one or more VEGFA cognate receptors alone. In some embodiments, the synergistic effect is increased phagocytosis of cancer cells by macrophages when comparing the effect of the combination versus either the agent that inhibits binding between CD47 and SIRPα or the agent that inhibits binding between VEGFA and one or more VEGFA cognate receptors alone. In some embodiments, the synergistic effect is increased or enhanced tumor burden reduction when comparing the effect of the combination versus either the agent that inhibits binding between CD47 and SIRPα or the agent that inhibits binding between VEGFA and one or more VEGFA cognate receptors alone. In some embodiments, the subject is human.

In a further aspect, provided is a kit comprising one or more unitary doses of: (a) an agent that inhibits binding between CD47 and SIRPα; and (b) an agent that inhibits binding between vascular endothelial growth factor A (VEGFA) and one or more VEGFA cognate receptors. In some embodiments, the one or more VEGFA cognate receptors are selected from fms related receptor tyrosine kinase 1 (FLT1, a.k.a., VEGFR1) and kinase insert domain receptor (KDR, a.k.a., VEGFR2). In some embodiments, the kit further comprises one or more unitary doses of a chemotherapy regimen. In some embodiments, the kit further comprises components (e.g., active agents) for a FOLFOX regimen, a FOLFIRI regimen, a XELIRI (a.k.a., CAPIRI) regimen, a FOLFOXIRI regimen, a XELOXIRI regimen or a FOLFIRINOX regimen. In some embodiments, the kit comprises components for a FOLFIRI regimen or a XELIRI regimen. In some embodiments, the kit comprises components for a FOLFOX regimen, a FOLFIRI regimen, a XELIRI (a.k.a., CAPIRI) regimen, a FOLFOXIRI regimen, a XELOXIRI regimen or a FOLFIRINOX regimen. In some embodiments, the kit comprises components for a FOLFIRI regimen or a XELIRI regimen. In some embodiments, the agent that inhibits binding between CD47 and SIRPα (e.g., magrolimab) and the agent that inhibits binding between vascular endothelial growth factor A (VEGFA) and one or more VEGFA cognate receptors (e.g., bevacizumab) are in separate containers. In some embodiments, the separate containers are selected from vials, ampoules and preloaded syringes. In some embodiments, the agent that inhibits binding between CD47 and SIRPα comprises an antibody that binds to CD47. In some embodiments, the antibody that binds to CD47 is selected from magrolimab, lemzoparlimab, letaplimab, ligufalimab, AO-176, simridarlimab (IBI-322), gentulizumab, ZL-1201, IMC-002, SRF-231, CC-90002 (a.k.a., INBRX-103), NI-1701 (a.k.a., TG-1801) and STI-6643. In some embodiments, the agent that inhibits binding between CD47 and SIRPα comprises an antibody that binds to SIRPα. In some embodiments, the antibody that binds to SIRPα is selected from GS-0189 (a.k.a., FSI-189), CC-95251, BI-765063 and APX-700. In some embodiments, the agent that inhibits binding between CD47 and SIRPα comprises a SIRPα-Fc fusion protein. In some embodiments, the SIRPα-Fc fusion protein is selected from ALX-148, TTI-621, TTI-622, JMT601 (CPO107) and SL-172154. In some embodiments, the kit further comprises one or more unitary doses of one or more therapeutic antibodies. In some embodiments, the kit further comprises one or more blockers or inhibitors of one or more T-cell inhibitory immune checkpoint proteins or receptors. In some embodiments, the kit comprises a proteinaceous (e.g., antibody) inhibitor of PD-L1 (CD274), PD-1 (PDCD1) or CTLA4. In some embodiments, the kit comprises a proteinaceous (e.g., antibody) inhibitor of PD-L1 (CD274) or PD-1 (PDCD1). In some embodiments, the proteinaceous (e.g., antibody) inhibitor of CTLA4 is selected from ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In some embodiments, the proteinaceous (e.g., antibody) inhibitor of programmed cell death 1 (PDCD1; NCBI Gene ID: 5133; CD279, PD-1, PD1) is selected from zimberelimab (AB122, GLS-010, WBP-3055), pembrolizumab (KEYTRUDA®, MK-3475, SCH900475), nivolumab (OPDIVO®, BMS-936558, MDX-1106), cemiplimab (LIBTAYO®; cemiplimab-rwlc, REGN-2810), pidilizumab (CT-011), AMG-404, MEDI0680 (AMP-514), spartalizumab (PDR001), tislelizumab (BGB-A317), toripalimab (JS-001), genolimzumab (CBT-501, APL-501, GB 226), SHR-1201, camrelizumab (SHR-1210), sintilimab (TYVYT®; IBI-308), dostarlimab (TSR-042, WBP-285), lambrolizumab (MK-3475); sasanlimab (PF-06801591), cetrelimab (JNJ-63723283), serplulimab (HLX-10), retifanlimab (MGA-012), balstilimab (AGEN2034), prolgolimab (BCD 100), budigalimab (ABBV-181), vopratelimab (JTX-4014), AK-103 (HX-008), AK-105, CS 1003, BI-754091, LZM-009, Sym-021, BAT-1306, PD1-PIK, tebotelimab (MGD013; PD-1/LAG-3), RO-7247669 (PD-1/LAG-3), FS-118 (LAG-3/PD-L1), RO-7121661 (PD 1/TIM-3), RG7769 (PD-1/TIM-3), PF-06936308 (PD 1/CTLA4), MGD-019 (PD-1/CTLA4), KN-046 (PD 1/CTLA4), XmAb-20717 (PD 1/CTLA4), AK-104 (CTLA4/PD-1) and MEDI-5752 (CTLA4/PD-1). In some embodiments, the proteinaceous (e.g., antibody) inhibitor of CD274 molecule (NCBI Gene ID: Gene ID: 29126; B7-H, B7H1, PD-L1) is selected from atezolizumab (TECENTRIQ®), avelumab (BAVENCIO®; MSB0010718C), envafolimab (ASC22), durvalumab (IMFINZI®; MEDI-4736), BMS-936559 (MDX1105), cosibelimab (CK-301), lodapolimab (LY 3300054), garivulimab (BGB A333), envafolimab (KN035), opucolimab (HLX 20), manelimab (BCD 135), CX-072, CBT-502 (TQB2450), MSB-2311, SHR-1316, sugemalimab (CS-1001; WBP3155), A167 (KL-A167, HBM 9167), STI-A1015 (IMC-001), FAZ-053, BMS-936559 (MDX1105), INCB086550, GEN-1046 (PD-L1/4-1BB), FPT-155 (CTLA4/PD-L1/CD28), M7824 (PD-L1/TGFβ-EC domain), CA-170 (PD-L1/VISTA), CDX-527 (CD27/PD-L1), LY-3415244 (TIM-3/PDL1), INBRX-105 (4-1BB/PDL1) and GNS-1480 (PD-L1/EGFR). In some embodiments, the kit further comprises co-administering an agonist of fms related receptor tyrosine kinase 3 (FLT3). In some embodiments, the agonist of FLT3 is selected from GS 3583 and CDX-301.

### BRIEF DESCRIPTION OF THE DRAWINGS

FIG. 1 illustrates a schema of the herein described clinical studies. 5-FU=5-fluorouracil; CRC=colorectal cancer; EU=European Union; FOLFIRI=5-fluorouracil, irinotecan, and leucovorin; KRAS=Kirsten rat sarcoma; KRASmt=KRAS mutation; KRASwt=KRAS wild type; ROW=rest of world; US=United States; FOLFIRI: irinotecan 180 mg/m², leucovorin 400 mg/m², fluorouracil 400 mg/m².

FIGS. 2A-2B illustrate that irinotecan upregulates the expression of prophagocytic receptors and promotes the in vitro phagocytosis of colorectal cancer cells. A. LS 174T cells were incubated with titrations of irinotecan for 24 hours, washed, and stained for CALR and PS. Flow cytometry was used to analyze and quantify the frequency of stained cells. The shaded areas indicate reported IC50 for each drug. B. LS-174T or HCT-116 cells were pretreated for 24 hours with irinotecan or oxaliplatin at the indicated concentration. The tumor cells were washed, CFSE labeled and cocultured with monocyte-derived macrophages at a 2:1 target to effector ratio for 2 hours. The phagocytic index, defined as the percentage of the macrophages that engulfed tumor cells out of total macrophages, was quantified using flow cytometry. 5F9, Magro=magrolimab; CALR=calreticulin; CFSE=carboxyfluorescein succinimidyl ester; hr=hour; IC50=half-maximal inhibitory concentration; MFI=median fluorescence intensity; PBS=phosphate buffered saline; PS=phosphatidyl serine; SN 38=irinotecan.

FIG. 3 illustrates colorectal cancer cells undergo growth inhibition with irinotecan and oxaliplatin. Tumor cells were treated with titrations of irinotecan, oxaliplatin, or vehicle control and monitored for growth after 24, 48, and 72 hours of drug treatment. Cell growth was indirectly determined using a commercially available reagent (Promega, Cell-titer Glo®) which measures intracellular adenosine triphosphate (ATP) levels and directly correlates to the number of live cells. Treated cells were normalized to dimethyl sulfoxide treated control samples to determine the percentage of growth inhibition. DMSO=dimethyl sulfoxide; Oxa=oxaliplatin; SN-38=irinotecan.

FIG. 4 illustrates combinations of irinotecan and magrolimab provide additive therapeutic efficacy in a colorectal cancer (CRC) chlordiazepoxide (CDX) Model. IP=intraperitoneal; Magro=magrolimab. NOD scid gamma mice were implanted subcutaneously with 3 million HT-29 cells. Mice were randomized into groups of 8 when mean tumor volumes reached 60 120 mm3 and treated with isotype or magrolimab by IP injection (250 μg, 3 times a week). Irinotecan (20 mg/kg, 3 times a week) and oxaliplatin (5 mg/kg, twice a week) was also administered by IP injection. Tumor volumes were generated using caliper measurements and reported for the duration of the study.

### DETAILED DESCRIPTION

1. Introduction

Provided are methods of treating previously treated advanced inoperable metastatic colorectal cancer in a subject comprising co-administering to the subject an effective amount of: (a) an agent that inhibits binding between CD47 and SIRPα (e.g., magrolimab); and (b) an agent that inhibits binding between vascular endothelial growth factor A (VEGFA) and one or more VEGFA cognate receptors (e.g., bevacizumab). Surprisingly combined administration of an agent that inhibits binding between a CD47 and SIRPα (e.g., magrolimab) and an agent that inhibits binding between vascular endothelial growth factor A (VEGFA) and one or more VEGFA cognate receptors (e.g., bevacizumab) results in synergistic (i.e., more than additive) phagocytosis of colorectal cancer cells and reduction in tumor growth.

### The Role of Chemotherapy in Chemotherapy in Metastatic Colorectal Cancer

Despite well-known genetic differences in the disease, chemotherapy treatment of CRC remains largely uniform. Patients with newly diagnosed mCRC are generally treated with 5-fluorouracil (5 FU) based regimens, such as FOLFOX (5-FU, oxaliplatin, and leucovorin [LV]) or FOLFIRI (5 FU, irinotecan, and leucovorin) alone or in combination with therapies that block EGFR or VEGF signaling.

A meta-analysis of 7 Phase 3 studies evaluating therapeutic regimens associating 5-FU/LV, oxaliplatin, and irinotecan demonstrated that patients receiving the 3 drugs, regardless of the first doublet regimen used, exhibited prolonged survival compared with patients receiving a single doublet (Grothey, et al., J Clin Oncol (2004) 22 (7):1209-14). However, capecitabine exhibited equivalence to 5-FU and represents a well-tolerated alternative association for irinotecan or oxaliplatin. After disease progression, a crossover second-line regimen associating fluoropyrimidine/oxaliplatin or fluoropyrimidine/irinotecan with another agent exhibited satisfactory response rates along with disease stabilized over a period of several months (Tournigand, et al., J Clin Oncol (2004) 22(2):229-37). The FOLFIRI and FOLFOX6 associations were evaluated for postprogression crossover first-line or second line treatment. Both arms exhibited similar response rates (first-line treatment: FOLFIRI 56% versus FOLFOX 54% and second-line treatment FOLFIRI 4% versus FOLFOX 15%). There were no significant differences in terms of progression-free survival (PFS) (first-line treatment: FOLFIRI 8.5 months versus FOLFOX 8.0 months and second-line treatment FOLFIRI 2.5 months versus FOLFOX 4.2 months) or of overall survival (OS) (FOLFIRI-FOLFOX: 21.5 months versus FOLFOX FOLFIRI 20.6 months).

### Rationale for Combination Therapy Co-Targeting CD47 and VEGFA

Cluster of differentiation 47 (CD47; NCBI Gene ID: 961) is a molecule mediating cancer cell evasion of innate immune surveillance. CD47 expression is a well-characterized mechanism by which cancer cells, including cancer stem cells, overcome phagocytosis due to intrinsic expression of prophagocytic “eat me” signals (Jaiswal, et al., Cell (2009) 138(2):271-85; Majeti, et al., Cell (2009) 138(2):286-99). The progression from normal cell to cancer cell involves changes in genes and gene expression that trigger programmed cell death and programmed cell removal (Chao, et al., Nat Rev Cancer. (2012) 12(1):58-67). Many of the steps in cancer progression subvert the multiple mechanisms of programmed cell death, and the expression of the dominant antiphagocytic signal, CD47, may represent an important checkpoint (Chao, et al., 2012, supra). Increased CD47 expression was identified first on leukemic stem cells in human acute myeloid leukemia (AML) (Majeti, et al., 2009, supra), and since then it has been found that CD47 expression is increased on the surface of cancer cells in a diverse set of human tumor types.

In mouse xenograft models, CD47-blocking monoclonal antibodies (mAbs) inhibit human xenograft tumor growth and metastasis by enabling the phagocytosis and elimination of cancer cells from various hematologic malignancies and solid tumors (Chao, et al., Cancer Res (2011) 71(4):1374-84; Chao, et al., Cell (2010) 142:699-713; Chao, et al., Blood (2011) 118 (18):4890-901; Edris, et al., Proc Natl Acad Sci USA (2012) 109(17):6656-61; Kim, et al., Proc Natl Acad Sci USA (2012) 109(17):6656-61; Majeti, et al., supra; Willingham, et al., Proc Natl Acad Sci USA (2012) 109(17):6662-7). Binding of CD47 expressed by cancer cells to its ligand, signal regulatory protein alpha (SIRPα), expressed on phagocytes leads to inhibition of tumor cell phagocytosis. Thus, blockade of the CD47 SIRPα-signaling pathway by an anti-CD47 antibody leads to phagocytosis and elimination of tumor cells. Selective targeting of tumor cells by an anti-CD47 antibody is due to the presence of prophagocytic signals expressed mainly on tumor cells and not on normal cell counterparts (Chao, et al., Sci Transl Med (2010) 2(63):63ra94). In addition, the anti-CD47 antibody can induce an anticancer T-cell response through cross-presentation of tumor antigens by macrophage and antigen-presenting cells after tumor cell phagocytosis (Liu, et al., Nat Med (2015) 21(10):1209-15, Tseng, et al., Proc Natl Acad Sci USA (2013) 110(27):11103-8).

Magrolimab is a humanized anti-CD47 mAb that blocks the interaction of CD47 with its receptor and enables phagocytosis of human cancer cells (Liu, et al., PLoS One. (2015) 10 (9):e0137345). The activity of magrolimab is primarily dependent on blocking CD47 binding to SIRPα and not on the recruitment of fragment crystallizable (Fc) dependent effector functions, although the presence of the immunoglobulin G4 (IgG4) Fc domain is required for its full activity. For this reason, magrolimab was engineered with a human IgG4 isotype that is relatively inefficient at recruiting Fc-dependent effector functions that might enhance toxic effects on normal CD47-expressing cells (Liu, et al., PLoS One. (2015), supra). Nonclinical studies using xenograft cancer models provide compelling evidence that magrolimab triggers phagocytosis and elimination of cancer cells from human solid tumors and hematologic malignancies. Based on this mechanism of action (MOA) and its potent nonclinical activity, magrolimab is being developed as a therapeutic candidate for solid tumors and hematologic malignancies.

Vascular endothelial growth factor A (VEGFA, NCBI Gene ID: 7422; also referenced herein as VEGF) is a factor involved in tumor angiogenesis, permeability, and tumor vascularization survival (Gerber, et al., Cancer Res (2005) 65 (3):671-80). The VEGF inhibition induces the destruction of recent neomicrovascularization and endothelial cell apoptosis (Erber, et al., FASEB J (2004) 18 (2):338-40; Ferrara, et al., Endocrine Rev (2004) 25(4):581-611).

One VEGF pathway inhibitor is bevacizumab, a humanized monoclonal antibody targeting circulatory VEGF and preventing tumor angiogenesis. The binding of bevacizumab with VEGF prevents the latter from binding with its endothelial cell membrane receptors (Flt-1 and kinase insert domain receptor (KDR)). The interaction of VEGF with its receptors, in angiogenesis models, induces tumor growth and neovessel formation. In addition to antiangiogenic properties, targeting the VEGF pathway with bevacizumab has been reported to reduce infiltration of immunosuppressive monocyte-derived suppressor cells and regulatory T cells as well as to upregulate cytotoxic T cells to the tumor microenvironment.

Randomized clinical studies have shown that bevacizumab combined with fluoropyrimidine-based chemotherapy is an effective treatment of patients with mCRC, and such combinations are now considered the standard treatment in the first-line and second-line settings (Hurwitz, et al., N Engl J Med (2004) 350(23):2335-42; Saltz, et al., J Clin Oncol (2008) 26(12):2013-9; Tebbutt, et al., J Clin Oncol (2010) 28(19):3191-8). Various studies have validated bevacizumab efficacy in patients who had no prior exposure or were treated with bevacizumab. In the bevacizumab-naive second line setting, longer PFS (7.3 versus 4.7 months, hazard ratio [HR]=0.61, P value<0.001) and OS (12.9 versus 10.8 months, HR=0.75, P value=0.0011), as well as a better response rate (22.7% versus 8.6%, P value=0.0001), were observed in the E3200 study comparing the combination of FOLFOX and bevacizumab with FOLFOX alone for patients with CRC who progressed after FOLFOX therapy (Giantonio, et al., 10 J Clin Oncol (2007) 25(12):1539-44).

The ML18147 study (Bennouna, et al., Lancet Oncol (2013) 14(1):29-37) and the BEBYP study (Masi, et al., Abstract LBA17, Ann Oncol. (2012) doi.org/10.1016/50923-7534(20)34318-0) indicate that continued VEGF inhibition with bevacizumab plus standard second-line chemotherapy (switched over from the first-line regimen) beyond first disease progression significantly prolongs OS and PFS in patients with mCRC. Continuation on bevacizumab for those who progressed after first-line chemotherapy was still helpful for PFS (5.7 versus 4.1 months, HR=0.68, P value<0.001) and OS (11.2 versus 9.9 months, HR=0.81, P value=0.0062) improvement compared with standard chemotherapy alone.

### Rationale for Dose Selection of Magrolimab

The rationale for the magrolimab dose proposed in this study originates from safety, efficacy, and PK/pharmacodynamics (PD) data and modeling and simulation analyses based on data obtained from all ongoing and completed clinical studies with magrolimab in patients with solid tumors, non-Hodgkin's lymphoma (NHL), and acute myeloid leukemia (AML)/myelodysplastic syndrome (MDS). Moreover, nonclinical studies have demonstrated activity against both human solid tumors (e.g., breast, ovarian, pancreas, colon, leiomyosarcoma, bladder, prostate, and others) and hematologic malignancies (acute myeloid leukemia (AML), acute lymphoblastic leukemia, non-Hodgkin's lymphoma (NHL), myeloma, myelodysplastic syndrome (MDS), among others).

In the first-in-human study of magrolimab in patients with solid tumors and lymphomas, after an initial priming dose of 1 mg/kg on the first day, magrolimab was tested as a monotherapy at weekly doses of up to 45 mg/kg. The use of an initial 1 mg/kg priming dose was integrated into the dosing regimen to mitigate the on-target anemia induced by CD47 blockade. An initial priming dose leads to elimination of aged RBCs that are sensitive to CD47 blockade and triggers reticulocytosis of young RBCs that are not affected by CD47 blockade (Chen, et al., Blood (ASH Annual Meeting Abstracts) (2018) 132 (Suppl 1):2327). Utilizing a priming dose leads to an initial, transient, and mild anemia that generally normalizes back to baseline over several weeks, even in the presence of repeated therapeutic doses of magrolimab (Advani, et al., N Engl J Med (2018) 379 (18):1711-21; Liu, et al., PLoS One. (2015) 10(9):e0137345; Sikic, et al., J Clin Oncol (2019) 37(12):946-53). Based on PK-PD modeling, a maintenance dose of 30 mg/kg every 2 weeks is expected to provide more than 90% occupancy of the CD47 receptor in peripheral blood and tumor tissues and thus is expected to provide maximal efficacy while maintaining adequate safety. In solid tumors where the combination therapy is given according to 3-week cycles, dosing of magrolimab every 3 weeks optimizes patient and caregiver convenience. Magrolimab 60 mg/kg every 3 weeks is predicted to provide a similar trough concentration and receptor occupancy (RO) as the 30 mg/kg every 2 weeks dose, the dose being used in Phase 3 studies in AML and MDS. Updated pharmacokinetic (PK) modeling from Study 5F9005 (NCT03248479) showed that the magrolimab dose of 45 mg/kg every 3 weeks was suboptimal compared to 30 mg/kg every 2 weeks and 60 mg/kg every 3 weeks dosing in maintaining trough concentration. Maintaining adequate trough concentration may be necessary for optimal efficacy considering that some patients may experience dose delays due to toxicity. Furthermore, the PK/pharmacodynamic (PD) modelling also indicates that at these extended interval dosing regimens, the RO will be maintained at maximal levels (>90%) in peripheral blood and tumor tissues. The proposed dosing regimen of magrolimab in this study is expected to have an acceptable safety profile based on the entirety of safety data in multiple oncology populations, both as a monotherapy and in combination with other tumor-targeted antibodies and chemotherapeutics.

2. Agent that Inhibits Binding Between CD47 and SIRPα

a. Antibody or Antigen-Binding Fragment Thereof that Binds to CD47

In various embodiments, the agent that inhibits binding between CD47 and SIRPα is an antibody or antigen-binding fragment thereof that binds to CD47 (a.k.a., IAP, MER6, OA3; NCBI Gene ID: 961; UniProt Q08722). In various embodiments, an antibody that binds to CD47 has an Fc having effector function. In various embodiments, an antibody that binds to CD47 is an IgG4 or an IgG1. Examples of anti-CD47 antibodies of use include without limitation magrolimab, lemzoparlimab, letaplimab, ligufalimab (AK117), AO-176, simridarlimab (IBI-322), gentulizumab, ZL-1201, IMC-002, SRF-231, CC-90002 (a.k.a., INBRX-103), NI-1701 (a.k.a., TG-1801), STI-6643 (Vx-1004), CNTO-7108, RCT-1938, RRx-001, DSP-107, VT-1021 and SGN-CD47M.

In various embodiments, the antibody targeting CD47 is a bi-specific antibody. Examples bi-specific antibodies targeting CD47, include without limitation IBI-322 (CD47/PD-L1), IMM-0306 (CD47/CD20), TJ-L1C4 (CD47/PD-L1), HX-009 (CD47/PD-1), PMC-122 (CD47/PD-L1), PT-217, (CD47/DLL3), IMM-26011 (CD47/FLT3), IMM-0207 (CD47/VEGF), IMM-2902 (CD47/HER2), BH29xx (CD47/PD-L1), IMM-03 (CD47/CD20), IMM-2502 (CD47/PD-L1), HMBD-004B (CD47/BCMA), HMBD-004A (CD47/CD33). Examples of anti-CD47antibodies, such as IBI-188, TJC-4, SHR-1603, HLX-24, LQ-001, IMC-002, ZL-1201, IMM-01, B6H12, GenSci-059, TAY-018, PT-240, 1F8-GMCSF, SY-102 and KD-015.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Kabat), respectively:
SEQ ID NOs: 1, 2, 3, 4, 5 and 6;
SEQ ID NOs: 7, 8, 9, 10, 11 and 12;
SEQ ID NOs: 13, 14, 15, 16, 17, and 18;
SEQ ID NOs: 19, 20, 21, 22, 23 and 24;
SEQ ID NOs: 210, 211, 212, 213, 214 and 215;
SEQ ID NOs: 216, 217, 218, 219, 220 and 221; or
SEQ ID NOs: 345, 346, 347, 348, 23 and 349.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to IMGT), respectively:
SEQ ID NOs: 25, 26, 27, 28, 29 and 6;
SEQ ID NOs: 30, 31, 32, 33, 34 and 12;
SEQ ID NOs: 35, 36, 37, 38, 39 and 18;
SEQ ID NOs: 40, 41, 42, 43, 44 and 24;
SEQ ID NOs: 222, 223, 224, 225, 226 and 215;
SEQ ID NOs: 227, 228, 229, 230, 231 and 221; or
SEQ ID NOs: 350, 351, 352, 353, 44 and 354.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Chothia), respectively:
SEQ ID NOs: 45, 46, 47, 48, 29 and 49;
SEQ ID NOs: 50, 51, 52, 53, 34 and 54;
SEQ ID NOs: 55, 56, 57, 58, 39 and 59;
SEQ ID NOs: 60, 61, 62, 62, 44 and 64;
SEQ ID NOs: 232, 233, 234, 235, 226 and 236;
SEQ ID NOs: 232, 237, 238, 239, 231 and 240; or
SEQ ID NOs: 355, 356, 357, 358, 44 and 359.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Honegger), respectively:
SEQ ID NOs: 65, 66, 67, 68, 69 and 49;
SEQ ID NOs: 70, 71, 72, 73, 74 and 54;
SEQ ID NOs: 75, 76, 77, 78, 79 and 59;
SEQ ID NOs: 80, 81, 82, 83, 84 and 64;
SEQ ID NOs: 241, 242, 243, 244, 245 and 246;
SEQ ID NOs: 247, 248, 249, 239, 250 and 251;
SEQ ID NOs: 342, 81, 82, 83, 84 and 64; or
SEQ ID NOs: 360, 361, 362, 363, 84 and 359.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 1, 2, 3, 4, 5 and 6 (according to Kabat);
SEQ ID NOs: 25, 26, 27, 28, 29 and 6 (according to IMGT);
SEQ ID NOs: 45, 46, 47, 48, 29 and 49 (according to Chothia); or
SEQ ID NOs: 65, 66, 67, 68, 69 and 49 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 7, 8, 9, 10, 11 and 12 (according to Kabat);
SEQ ID NOs: 30, 31, 32, 33, 34 and 12 (according to IMGT);
SEQ ID NOs: 50, 51, 52, 53, 34 and 54 (according to Chothia); or
SEQ ID NOs: 70, 71, 72, 73, 74 and 54 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 13, 14, 15, 16, 17, and 18 (according to Kabat);
SEQ ID NOs: 35, 36, 37, 38, 39 and 18 (according to IMGT);
SEQ ID NOs: 55, 56, 57, 58, 39 and 59 (according to Chothia); or
SEQ ID NOs: 80, 81, 82, 83, 84 and 64 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 19, 20, 21, 22, 23 and 24 (according to Kabat);
SEQ ID NOs: 40, 41, 42, 43, 44 and 24 (according to IMGT);
SEQ ID NOs: 60, 61, 62, 62, 44 and 64 (according to Chothia); or
SEQ ID NOs: 80, 81, 82, 83, 84 and 64 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 210, 211, 212, 213, 214 and 215 (according to Kabat);
SEQ ID NOs: 222, 223, 224, 225, 226 and 215 (according to IMGT);
SEQ ID NOs: 232, 233, 234, 235, 226 and 236 (according to Chothia); or
SEQ ID NOs: 241, 242, 243, 244, 245 and 246 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 216, 217, 218, 219, 220 and 221 (according to Kabat);
SEQ ID NOs: 227, 228, 229, 230, 231 and 221 (according to IMGT);
SEQ ID NOs: 232, 237, 238, 239, 231 and 240 (according to Chothia); or
SEQ ID NOs: 247, 248, 249, 239, 250 and 251 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 339, 20, 21, 22, 23 and 24 (according to Kabat);
SEQ ID NOs: 340, 41, 42, 43, 44 and 24 (according to IMGT);
SEQ ID NOs: 341, 61, 62, 63, 44 and 64 (according to Chothia); or
SEQ ID NOs: 342, 81, 82, 83, 84 and 64 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 345, 346, 347, 348, 23 and 349 (according to Kabat);
SEQ ID NOs: 350, 351, 352, 353, 44 and 354 (according to IMGT);
SEQ ID NOs: 355, 356, 357, 358, 44 and 359 (according to Chothia); or
SEQ ID NOs: 360, 361, 362, 363, 84 and 359 (according to Honegger).

In various embodiments, the antibody targeting CD47 comprises a VH and a VL comprising the amino acid sequences set forth, respectively, or comprise amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth, respectively, in:
SEQ ID NOs: 85 and 86;
SEQ ID NOs: 87 and 88;
SEQ ID NOs: 89 and 90;
SEQ ID NOs: 91 and 92;
SEQ ID NOs: 252 and 253;
SEQ ID NOs: 254 and 255;
SEQ ID NOs: 343 and 344; or
SEQ ID NOs: 364 and 365. Sequence identity can be determined according to the BLAST algorithm (blast.ncbi.nlm.nih.gov/Blast.cgi), using default settings.

Amino acid sequences of CDRs and variable regions (VH/VL) of illustrative anti-CD47 antibodies that can be used in the present methods are described in Tables A1, A2, A3, A4 and B.

CDRs for illustrative anti-CD47 binding antibodies (Kabat)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 1 | NYNMH | TIYPGNDDTSYNQKFKD | GGYRAMDY | RSSQSIVYSNGNTYLG | KVSNRFS | FQGSHVPYT |
| | SEQ ID NO: 1 | SEQ ID NO: 2 | SEQ ID NO: 3 | SEQ ID NO: 4 | SEQ ID | SEQ ID NO: 6 |
| | | | | | NO: 5 | |
| 2 | DYYIN | RIYPGIGNTYYNKKFKG | GHYGRGMDY | KSSQSLLNSIDQKNYLA | FASTKES | QQHYSTPWT |
| | SEQ ID NO: 7 | SEQ ID NO: 8 | SEQ ID NO: 9 | SEQ ID NO: 10 | SEQ ID | SEQ ID NO: 12 |
| | | | | | NO: 11 | |
| 3 | RAWMN | RIKRKTDGETTDYAAPV | SNRAFDI | KSSQSVLYAGNNRNYLA | QASTRAS | QQYYTPPLA |
| | SEQ ID NO: 13 | KG | SEQ ID NO: 15 | SEQ ID NO: 16 | SEQ ID | SEQ ID NO: 18 |
| | | SEQ ID NO: 14 | | | NO: 17 | |
| 4 | SYYWSW | YIYYSGSTNYNPSLKS | GKTGSAA | RASQGISRWLA | AASSLQS | QQTVSFPIT |
| | SEQ ID NO: 19 | SEQ ID NO: 20 | SEQ ID NO: 21 | SEQ ID NO: 22 | SEQ ID | SEQ ID NO: 24 |
| | | | | | NO: 23 | |
| 51 | SYWMN | MIDPSDSETHNAQKFQG | LYRWYFDV | RASEIVGTYVS | GASNRYT | GQSYNFPYT |
| | SEQ ID NO: 210 | SEQ ID NO: 211 | SEQ ID NO: 212 | SEQ ID NO: 213 | SEQ ID | SEQ ID NO: 215 |
| | | | | | NO: 214 | |
| 52 | SYYMH | IINPSGGSTSYAQKFQG | STLWFSEFDY | SGTSSDVGGHNYVS | DVTKRPS | LSYAGSRVY |
| | SEQ ID NO: 216 | SEQ ID NO: 217 | SEQ ID NO: 218 | SEQ ID NO: 219 | SEQ ID | SEQ ID NO: 221 |
| | | | | | NO: 220 | |
| 86 | HYYWS | YIYYSGSTNYNPSLKS | GKTGSAA | RASQGISRWLA | AASSLQS | QQTVSFPIT |
| | SEQ ID NO: 339 | SEQ ID NO: 20 | SEQ ID NO: 21 | SEQ ID NO: 22 | SEQ ID | SEQ ID NO: 24 |
| | | | | | NO: 23 | |
| 91 | SYAMS | AISGSGGSTYYADSVKG | SYGAFDY | RASQSISSYLN | AASSLOS | QQMHPRAPKT |
| | SEQ ID NO: 345 | SEQ ID NO: 346 | SEQ ID NO: 347 | SEQ ID NO: 348 | SEQ ID | SEQ ID NO: 349 |
| | | | | | NO: 23 | |

TABLE A2
CDRs for illustrative anti-CD47 binding antibodies (IMGT)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 5 | GYTFTNYN | IYPGNDDT | ARGGYRAMDY | QSIVYSNGNTY | KVS | FQGSHVPYT |
| | SEQ ID NO: 25 | SEQ ID NO: 26 | SEQ ID NO: 27 | SEQ ID NO: 28 | SEQ ID | SEQ ID NO: 6 |
| | | | | | NO: 29 | |
| 6 | GYSFTDYY | IYPGIGNT | ARGHYGRGMDY | QSLLNSIDQKNY | FAS | QQHYSTPWT |
| | SEQ ID NO: 30 | SEQ ID NO: 31 | SEQ ID NO: 32 | SEQ ID NO: 33 | SEQ ID | SEQ ID NO: 12 |
| | | | | | NO: 34 | |
| 7 | GLTFERAW | IKRKTDGETT | AGSNRAFDI | QSVLYAGNNRNY | QAS | QQYYTPPLA |
| | SEQ ID NO: 35 | SEQ ID NO: 36 | SEQ ID NO: 37 | SEQ ID NO: 38 | SEQ ID | SEQ ID NO: 18 |
| | | | | | NO: 39 | |
| 8 | GGSISSYY | IYYSGST | ARGKTGSAA | QGISRW | AAS | QQTVSFPIT |
| | SEQ ID NO: 40 | SEQ ID NO: 41 | SEQ ID NO: 42 | SEQ ID NO: 43 | SEQ ID | SEQ ID NO: 24 |
| | | | | | NO: 44 | |
| 53 | GYTFTSYW | IDPSDSET | ARLYRWYFDV | EIVGTY | GAS | GQSYNFPYT |
| | SEQ ID NO: 222 | SEQ ID NO: 223 | SEQ ID NO: 224 | SEQ ID NO: 225 | SEQ ID | SEQ ID NO: 215 |
| | | | | | NO: 226 | |
| 54 | GYTFTSYY | INPSGGST | ARSTLWF SEFDY | SSDVGGHNY | DVT | LSYAGSRVY |
| | SEQ ID NO: 227 | SEQ ID NO: 228 | SEQ ID NO: 229 | SEQ ID NO: 230 | SEQ ID | SEQ ID NO: 221 |
| | | | | | NO:231 | |
| 87 | GGSIEHYY | IYYSGST | ARGKTGSAA | QGISRW | AAS | QQTVSFPIT |
| | SEQ ID NO: 340 | SEQ ID NO: 41 | SEQ ID NO: 42 | SEQ ID NO: 43 | SEQ ID | SEQ ID NO: 24 |
| | | | | | NO: 44 | |
| 91 | GFTFSSYA | ISGSGGST | AKSYGAFDY | QSISSY | AAS | QQMHPRAPKT |
| | SEQ ID NO: 350 | SEQ ID NO: 351 | SEQ ID NO: 352 | SEQ ID NO: 353 | SEQ ID | SEQ ID NO: 354 |
| | | | | | NO: 44 | |

TABLE A3
CDRs for illustrative anti-CD47 binding antibodies (Chothia)

| Ab | | | | | | |
|---|---|---|---|---|---|---|
| Name | VH - CDR1 | VH - CDR2 | VH - CDR3 | VL - CDR1 | VL - CDR2 | VL - CDR3 |
| 9 | GYTFTNY | PGND | GYRAMD | SQSIVYSNGNTY | KVS | GSHVPY |
| | SEQ ID NO: 45 | SEQ ID NO: 46 | SEQ ID NO: 47 | SEQ ID NO: 48 | SEQ ID NO: 29 | SEQ ID NO: 49 |
| 10 | GYSFTDY | PGIG | HYGRGMD | SQSLLNSIDQKNY | FAS | HYSTPW |
| | SEQ ID NO: 50 | SEQ ID NO: 51 | SEQ ID NO: 52 | SEQ ID NO: 53 | SEQ ID NO: 34 | SEQ ID NO: 54 |
| 11 | GLTFERA | RKTDGE | NRAFD | SQSVLYAGNNRNY | QAS | YYTPPL |
| | SEQ ID NO: 55 | SEQ ID NO: 56 | SEQ ID NO: 57 | SEQ ID NO: 58 | SEQ ID NO: 39 | SEQ ID NO: 59 |
| 12 | GGSISSY | YSG | KTGSA | SQGISRW | AAS | TVSFPI |
| | SEQ ID NO: 60 | SEQ ID NO: 61 | SEQ ID NO: 62 | SEQ ID NO: 63 | SEQ ID NO: 44 | SEQ ID NO: 64 |
| 55 | GYTFTSY | PSDS | YRWYFD | SEIVGTY | GAS | SYNFPY |
| | SEQ ID NO: 232 | SEQ ID NO: 233 | SEQ ID NO: 234 | SEQ ID NO: 235 | SEQ ID NO: 226 | SEQ ID NO: 236 |
| 56 | GYTFTSY | PSGG | TLWFSEFD | GTSSDVGGHNY | DVT | YAGSRV |
| | SEQ ID NO: 232 | SEQ ID NO: 237 | SEQ ID NO: 238 | SEQ ID NO: 239 | SEQ ID NO: 231 | SEQ ID NO: 240 |
| 88 | GGSIEHY | YSG | KTGSA | SQGISRW | AAS | TVSFPI |
| | SEQ ID NO: 341 | SEQ ID NO: 61 | SEQ ID NO: 62 | SEQ ID NO: 63 | SEQ ID NO: 44 | SEQ ID NO: 64 |
| 92 | GFTFSSY | GSGG | YGAFD | SQSISSY | AAS | MHPRAPK |
| | SEQ ID NO: 355 | SEQ ID NO: 356 | SEQ ID NO: 357 | SEQ ID NO: 358 | SEQ ID NO: 44 | SEQ ID NO: 359 |

TABLE A4
CDRs for illustrative anti-CD47 binding antibodies (Honegger)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 13 | ASGYTFTNYN | IYPGNDDTSYNQKFKDR | GGYRAMD | SSQSIVYSNGNTY | KVSNRFSGVPDR | GSHVPY |
| | SEQ ID NO: 65 | SEQ ID NO: 66 | SEQ ID NO: 67 | SEQ ID NO: 68 | SEQ ID NO: 69 | SEQ ID |
| | | | | | | NO: 49 |
| 14 | ASGYSFTDYY | IYPGIGNTYYNKKFKGR | GHYGRGMD | SSQSLLNSIDQKNY | FASTKESGVPDR | HYSTPW |
| | SEQ ID NO: 70 | SEQ ID NO: 71 | SEQ ID NO: 72 | SEQ ID NO: 73 | SEQ ID NO: 74 | SEQ ID |
| | | | | | | NO: 54 |
| 15 | ASGLTFERAW | IKRKTDGETTDYAAPVK | SNRAFD | SSQSVLYAGNNRNY | QASTRASGVPDR | YYTPPL |
| | SEQ ID NO: 75 | GR | SEQ ID NO: 77 | SEQ ID NO: 78 | SEQ ID NO: 79 | SEQ ID |
| | | SEQ ID NO: 76 | | | | NO: 59 |
| 16 | VSGGSISSYY | IYYSGSTNYNPSLKSR | GKTGSA | ASQGISRW | AASSLQSGVPSR | TVSFPI |
| | SEQ ID NO: 80 | SEQ ID NO: 81 | SEQ ID NO: 82 | SEQ ID NO: 83 | SEQ ID NO: 84 | SEQ ID |
| | | | | | | NO: 64 |
| 57 | ASGYTFTSYW | IDPSDSETHNAQKFQGK | LYRWYFD | ASEIVGTY | GASNRYTGVPAR | SYNFPY |
| | SEQ ID NO: 241 | SEQ ID NO: 242 | SEQ ID NO: 243 | SEQ ID NO: 244 | SEQ ID NO: 245 | SEQ ID |
| | | | | | | NO: 246 |
| 58 | ASGYTFTSYY | INPSGGSTSYAQKFQGR | STLWFSEFD | GTSSDVGGHNY | DVTKRPSGVPDR | YAGSRVY |
| | SEQ ID NO: 247 | SEQ ID NO:248 | SEQ ID NO: 249 | SEQ ID NO: 239 | SEQ ID NO: 250 | SEQ ID |
| | | | | | | NO: 251 |
| 89 | VSGGSIEHYY | IYYSGSTNYNPSLKSR | GKTGSA | ASQGISRW | AASSLQSGVPSR | TVSFPI |
| | SEQ ID NO: 342 | SEQ ID NO: 81 | SEQ ID NO: 82 | SEQ ID NO: 83 | SEQ ID NO: 84 | SEQ ID |
| | | | | | | NO: 64 |
| 93 | ASGFTFSSYA | ISGSGGSTYYADSVKGR | SYGAFD | ASQSISSY | AASSLQSGVPSR | MHPRAPK |
| | SEQ ID NO: 360 | SEQ ID NO: 361 | SEQ ID NO: 362 | SEQ ID NO: 363 | SEQ ID NO: 84 | SEQ ID |
| | | | | | | NO: 359 |

VH/VL for illustrative anti-CD47 binding antibodies

| Ab | | |
|---|---|---|
| Name | VH | VL |
| 17 | SEQ ID NO: 85 | SEQ ID NO: 86 |
| | QVQLVQSGAEVKKPGASVKVSCKASGYTFTNYNMHWVRQA | DIVMTQSPLSLPVTPGEPASISCRSSQSIVYSNGNTYL |
| | PGQRLEWMGTIYPGNDDTSYNQKFKDRVTITADTSASTAY | GWYLQKPGQSPQLLIYKVSNRFSGVPDRFSGSGSGTDF |
| | MELSSLRSEDTAVYYCARGGYRAMDYWGQGTLVTVSS | TLKISRVEAEDVGVYYCFQGSHVPYTFGQGTKLEIK |
| 18 | SEQ ID NO: 87 | SEQ ID NO: 88 |
| | QVQLVQSGAEVKKPGASVKVSCKASGYSFTDYYINWVRQA | DIVMTQSPDSLAVSLGERATINCKSSQSLLNSIDQKNY |
| | PGQGLEWMGRIYPGIGNTYYNKKFKGRVTITRDTSASTAY | LAWYQQKPGQPPKLLIYFASTKESGVPDRFSGSGSGTD |
| | MELSSLRSEDTAVYYCARGHYGRGMDYWGQGTLVTVSS | FTLTISGLQAEDVAVYFCQQHYSTPWTFGGGTKVEIR |
| 19 | SEQ ID NO: 89 | SEQ ID NO: 90 |
| | EVQLVESGGGLVKPGGSLRLSCAASGLTFERAWMNWVRQA | DIVMTQSPDSLAVSLGERATINCKSSQSVLYAGNNRNY |
| | PGKGLEWVGRIKRKTDGETTDYAAPVKGRFSISRDDSKNT | LAWYQQKPGQPPKLLINQASTRASGVPDRFSGSGSGTE |
| | LYLQMNSLKTEDTAVYYCAGSNRAFDIWGQGTMVTVSS | FTLIISSLQAEDVAIYYCQQYYTPPLAFGGGTKLEIK |
| 20 | SEQ ID NO: 91 | SEQ ID NO: 92 |
| | QVQLQESGPGLVKPSETLSLTCTVSGGSISSYYWSWIRQP | DIQMTQSPSSVSASVGDRVTITCRASQGISRWLAWYQQ |
| | PGKGLEWIGYIYYSGSTNYNPSLKSRVTISVDTSKNQFSL | KPGKAPKLLIYAASSLQSGVPSRFSGSGSGTDFTLTIS |
| | KLSSVTAADTAVYYCARGKTGSAAWGQGTLVTVSS | SLQPEDFATYYCQQTVSFPITFGGGTKVEIK |
| 59 | SEQ ID NO: 252 | SEQ ID NO: 253 |
| | QVQLVQSGAEVVKPGASVKLSCKASGYTFTSYWMNWVRQR | NIVMTQSPATMSMSPGERVTLSCRASEIVGTYVSWFQQ |
| | PGQGLEWIGMIDPSDSETHNAQKFQGKATLTVDKSTSTAY | KPGQAPRLLIYGASNRYTGVPARFSGSGSGTDFTLTIS |
| | MHLSSLRSEDTAVYYCARLYRWYFDVWGAGTTVTVSS | SVQPEDLADYHCGQSYNFPYTFGGGTKLEIK |
| 60 | SEQ ID NO: 254 | SEQ ID NO: 255 |
| | QVQLVQSGAEVKKPGASVKVSCKASGYTFTSYYMHWVRQA | QSVLTQPSSVSASPGQSITISCSGTSSDVGGHNYVSWY |
| | PGQGLEWMGIINPSGGSTSYAQKFQGRVTMTRDTSTSTVY | QQHPGKAPKLMIYDVTKRPSGVPDRFSGSKSGNTASLT |
| | MELSSLRSEDTAVYYCARSTLWFSEFDYWGQGTLVTVSS | VSGLQAEDEADYYCLSYAGSRVYVFGTGTKLTVL |
| 90 | SEQ ID NO: 343 | SEQ ID NO: 344 |
| | QVQLQESGPGLVKPSETLSLTCTVSGGSIEHYYWSWIRQP | DIQMTQSPSSVSASVGDRVTITCRASQGISRWLAWYQQ |
| | PGKGLEWIGYIYYSGSTNYNPSLKSRVTISVDTSKNQFSL | KPGKAPKLLIYAASSLQSGVPSRFSGSGSGTDFTLTIS |
| | KLSSVTAADTAVYYCARGKTGSAAWGQGTLVTVSS | SLQPEDFATYYCQQTVSFPITFGGGTKVEIK |
| 94 | SEQ ID NO: 364 | SEQ ID NO: 365 |
| | EVQLLESGGGLVQPGGSLRLSCAASGFTFSSYAMSWVRQA | DIQMTQSPSSLSASVGDRVTITCRASQSISSYLNWYQQ |
| | PGKGLEWVSAISGSGGSTYYADSVKGRFTISRDNSKNTLY | KPGKAPKLLIYAASSLQSGVPSRFSGSGSGTDFTLTIS |
| | LQMNSLRAEDTAVYYCAKSYGAFDYWGQGTLTVSS | SLQPEDFATYYCQQMHPRAPKTFGQGTKVEIK |

Additional anti-CD47 antibodies of use in the present methods include those described in WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188 and WO2020009725.

b. Antibody or Antigen-Binding Fragment Thereof that Binds to SIRPα

In various embodiments, the agent that inhibits binding between CD47 and SIRPα CD47 is an antibody or antigen-binding fragment thereof that binds to signal regulatory protein alpha (SIRPα) (NCBI Gene ID: 140885; UniProt P78324). Illustrative antibodies that bind to SIRPα include without limitation GS-0189 (FSI-189), ES-004, BI765063, ADU1805, and CC-95251.

In certain embodiments, an antibody can comprise one or more CDRs of 1H9. In some embodiments, an antibody can comprise all CDRs of 1H9. In some embodiments, an antibody can comprise one or more variable sequences of 1H9. In some embodiments, an antibody can comprise each variable sequence of 1H9. In some embodiments, an antibody can comprise the heavy chain of 1H9. In some embodiments, an antibody can comprise the light chain of 1H9. In some embodiments, an antibody can comprise the heavy chain and the light chain of 1H9. In some embodiments, an antibody is 1H9.

In certain embodiments, an antibody can comprise one or more CDRs of 3C2. In some embodiments, an antibody can comprise all CDRs of 3C2. In some embodiments, an antibody can comprise one or more variable sequences of 3C2. In some embodiments, an antibody can comprise each variable sequence of 3C2. In some embodiments, an antibody can comprise the heavy chain of 3C2. In some embodiments, an antibody can comprise the light chain of 3C2. In some embodiments, an antibody can comprise the heavy chain and the light chain of 3C2. In some embodiments, an antibody is 3C2.

In some embodiments, an antibody can comprise one or more CDRs of 9B11. In some embodiments, an antibody can comprise all CDRs of 9B11. In some embodiments, an antibody can comprise one or more variable sequences of 9B11. In some embodiments, an antibody can comprise each variable sequence of 9B11. In some embodiments, an antibody can comprise the heavy chain of 9B11. In some embodiments, an antibody can comprise the light chain of 9B11. In some embodiments, an antibody can comprise the heavy chain and the light chain of 9B11. In some embodiments, an antibody is 9B11.

In some embodiments, an antibody can comprise one or more CDRs of 7E11. In some embodiments, an antibody can comprise all CDRs of 7E11. In some embodiments, an antibody can comprise one or more variable sequences of 7E11. In some embodiments, an antibody can comprise each variable sequence of 7E11. In some embodiments, an antibody can comprise the heavy chain of 7E11. In some embodiments, an antibody can comprise the light chain of 7E11. In some embodiments, an antibody can comprise the heavy chain and the light chain of 7E11. In some embodiments, an antibody is 7E11.

Additional anti-SIRPα antibodies of use in the present methods include those described in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170, WO2020068752 and WO2020088580.

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences (according to Kabat), respectively:
SEQ ID NOs: 93, 94, 95, 96, 97 and 98;
SEQ ID NOs: 99, 100, 101, 102, 103 and 104;
SEQ ID NOs: 99, 100, 105, 102, 103 and 106;
SEQ ID NOs: 107, 108, 109, 110, 111 and 112;
SEQ ID NOs: 113, 114, 115, 116, 117 and 118; or
SEQ ID NOs: 119, 120, 121, 122, 123 and 124.

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences (according to IMGT), respectively:
SEQ ID NOs: 125, 126, 127, 128, 129 and 98;
SEQ ID NOs: 125, 130, 131, 132, 29 and 104;
SEQ ID NOs: 125, 130, 133, 132, 29 and 106;
SEQ ID NOs: 134, 135, 136, 137, 138 and 112;
SEQ ID NOs: 139, 130, 140, 141, 142 and 118; or
SEQ ID NOs: 143, 144, 145, 146, 44 and 124.

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences (according to Chothia), respectively:
SEQ ID NOs: 147, 148, 149, 150, 129 and 151;
SEQ ID NOs: 147, 152, 153, 154, 29 and 155;
SEQ ID NOs: 147, 152, 156, 154, 29 and 157;
SEQ ID NOs: 158, 159, 160, 161, 138 and 162;
SEQ ID NOs: 163, 152, 164, 165, 142 and 166; or
SEQ ID NOs: 167, 168, 169, 170, 44 and 171.

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences (according to Honegger), respectively:
SEQ ID NOs: 172, 173, 174, 175, 176 and 151;
SEQ ID NOs: 172, 177, 178, 179, 180 and 155;
SEQ ID NOs: 172, 181, 182, 179, 180 and 157;
SEQ ID NOs: 183, 184, 185, 186, 187 and 162;
SEQ ID NOs: 188, 189, 190, 191, 192 and 166; or
SEQ ID NOs: 193, 194, 195, 196, 197 and 171.

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences, respectively:
SEQ ID NOs: 93, 94, 95, 96, 97 and 98 (according to Kabat);
SEQ ID NOs: 125, 126, 127, 128, 129 and 98 (according to IMGT);
SEQ ID NOs: 147, 148, 149, 150, 129 and 151 (according to Chothia); or
SEQ ID NOs: 172, 173, 174, 175, 176 and 151 (according to Honegger).

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences, respectively:
SEQ ID NOs: 99, 100, 101, 102, 103 and 104 (according to Kabat);
SEQ ID NOs: 125, 130, 131, 132, 29 and 104 (according to IMGT);
SEQ ID NOs: 147, 152, 153, 154, 29 and 155 (according to Chothia); or
SEQ ID NOs: 172, 177, 178, 179, 180 and 155 (according to Honegger).

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences, respectively:
SEQ ID NOs: 99, 100, 105, 102, 103 and 106 (according to Kabat);
SEQ ID NOs: 125, 130, 133, 132, 29 and 106 (according to IMGT);
SEQ ID NOs: 147, 152, 156, 154, 29 and 157 (according to Chothia); or
SEQ ID NOs: 172, 181, 182, 179, 180 and 157 (according to Honegger).

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences, respectively:
SEQ ID NOs: 107, 108, 109, 110, 111 and 112 (according to Kabat);
SEQ ID NOs: 134, 135, 136, 137, 138 and 112 (according to IMGT);
SEQ ID NOs: 158, 159, 160, 161, 138 and 162 (according to Chothia); or
SEQ ID NOs: 183, 184, 185, 186, 187 and 162 (according to Honegger).

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences, respectively:
SEQ ID NOs: 113, 114, 115, 116, 117 and 118 (according to Kabat);
SEQ ID NOs: 139, 130, 140, 141, 142 and 118 (according to IMGT);
SEQ ID NOs: 163, 152, 164, 165, 142 and 166 (according to Chothia); or
SEQ ID NOs: 188, 189, 190, 191, 192 and 166 (according to Honegger).

In various embodiments, the antibody targeting SIRPα comprises a VH-CDR1, a VH-CDR2, a VH-CDR3, a VL-CDR1, a VL-CDR2 and a VL-CDR3 comprising the following amino acid sequences, respectively:
SEQ ID NOs: 119, 120, 121, 122, 123 and 124 (according to Kabat);
SEQ ID NOs: 143, 144, 145, 146, 44 and 124 (according to IMGT);
SEQ ID NOs: 167, 168, 169, 170, 44 and 171 (according to Chothia); or
SEQ ID NOs: 193, 194, 195, 196, 197 and 171 (according to Honegger).

In various embodiments, the antibody targeting SIRPα comprises a VH and a VL comprising the amino acid sequences set forth, respectively, or comprise amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth, respectively, in:
SEQ ID NOs: 198 and 199;
SEQ ID NOs: 200 and 201;
SEQ ID NOs: 202 and 203;
SEQ ID NOs: 204 and 205;
SEQ ID NOs: 206 and 207; or
SEQ ID NOs: 208 and 209. Sequence identity can be determined according to the BLAST algorithm (blast.ncbi.nlm.nih.gov/Blast.cgi), using default settings.

Amino acid sequences of CDRs and variable regions (VH/VL) of illustrative anti-SIRPα antibodies that can be used in the present methods are described in Tables C1, C2, C3, C4 and D.

TABLE C1
CDRs for illustratvie anti-SIRPα binding antibodies (Kabat)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 21 | SYWIT | DIYPGSGSTNHIEKFKS | GYGSSYGYFDY | RASENTYSYLA | TAKTLAE | QHQYGPPFT |
| | SEQ ID | SEQ ID NO: 94 | SEQ ID NO: 95 | SEQ ID NO: 96 | SEQ ID | SEQ ID NO: 98 |
| | NO: 93 | | | | NO: 97 | |
| 22 | SYWMH | NIDPSDSDTHYNQKFKD | GYSKYYAMDY | RSSQSIVHSYGNT | KVSNRFS | FQGSHVPYT |
| | SEQ ID | SEQ ID NO: 100 | SEQ ID | YLE | SEQ ID | SEQ ID |
| | NO: 99 | | NO: 101 | SEQ ID NO: 102 | NO: 103 | NO: 104 |
| 23 | SYWMH | NIDPSDSDTHYNQKFKD | YGNYGENAMDY | RSSQSIVHSYGNT | KVSNRFS | FQGSHVPFT |
| | SEQ ID | SEQ ID NO: 100 | SEQ ID | YLE | SEQ ID | SEQ ID |
| | NO: 99 | | NO: 105 | SEQ ID NO: 102 | NO: 103 | NO: 106 |
| 24 | DYYIH | RIDPEDGETKYAPKFQG | GGFAY | ASSSVSSSYLY | STSNLAS | HQWSSHPYT |
| | SEQ ID | SEQ ID NO: 108 | SEQ ID | SEQ ID NO: 110 | SEQ ID | SEQ ID |
| | NO: 107 | | NO: 109 | | NO: 111 | NO: 112 |
| 25 | SYWVH | NIDPSDSDTHYSPSFQG | GGTGTLAYFAY | RSSQSLVHSYGNT | RVSNRFS | FQGTHVPYT |
| | SEQ ID | SEQ ID NO: 114 | SEQ ID | YLY | SEQ ID | SEQ ID |
| | NO: 113 | | NO: 115 | SEQ ID NO: 116 | NO: 117 | NO: 118 |
| 26 | GYGIS | WISAYGGETNYAQKLQG | EAGSSWYDFDL | RASQGISSWLA | AASNLQS | QQGASFPIT |
| | SEQ ID | SEQ ID NO: 120 | SEQ ID | SEQ ID NO: 122 | SEQ ID | SEQ ID |
| | NO: 119 | | NO: 121 | | NO: 123 | NO: 124 |

TABLE C2
CDRs for illustrative anti-SIRPα binding antibodies (IMGT)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 27 | GYTFTSYW | IYPGSGST | ATGYGSSYGYFDY | ENTYSY | TAK | QHQYGPPFT |
| | SEQ ID NO: 125 | SEQ ID NO: 126 | SEQ ID NO: 127 | SEQ ID NO: 128 | SEQ ID | SEQ ID NO: 98 |
| | | | | | NO: 129 | |
| 28 | GYTFTSYW | IDPSDSDT | ARGYSKYYAMDY | QSIVHSYGNTY | KVS | FQGSHVPYT |
| | SEQ ID NO: 125 | SEQ ID NO: 130 | SEQ ID NO: 131 | SEQ ID NO: 132 | SEQ ID | SEQ ID NO: 104 |
| | | | | | NO: 29 | |
| 29 | GYTFTSYW | IDPSDSDT | ASYGNYGENAMDY | QSIVHSYGNTY | KVS | FQGSHVPFT |
| | SEQ ID NO: 125 | SEQ ID NO: 130 | SEQ ID NO: 133 | SEQ ID NO: 132 | SEQ ID | SEQ ID NO: 106 |
| | | | | | NO: 29 | |
| 30 | GFNIKDYY | IDPEDGET | AKGGFAY | SSVSSSY | STS | HQWSSHPYT |
| | SEQ ID NO: 134 | SEQ ID NO: 135 | SEQ ID NO: 136 | SEQ ID NO: 137 | SEQ ID | SEQ ID NO: 112 |
| | | | | | NO: 138 | |
| 31 | GYSFTSYW | IDPSDSDT | VRGGTGTLAYFAY | QSLVHSYGNTY | RVS | FQGTHVPYT |
| | SEQ ID NO: 139 | SEQ ID NO: 130 | SEQ ID NO: 140 | SEQ ID NO: 141 | SEQ ID | SEQ ID NO: 118 |
| | | | | | NO: 142 | |
| 32 | GYTFRGYG | ISAYGGET | AREAGSSWYDFDL | QGISSW | AAS | QQGASFPIT |
| | SEQ ID NO: 143 | SEQ ID NO: 144 | SEQ ID NO: 145 | SEQ ID NO: 146 | SEQ ID | SEQ ID NO: 124 |
| | | | | | NO: 44 | |

TABLE C3
CDRs for illustrative anti-SIRPα binding antibodies (Chothia)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 33 | GYTFTSY | PGSG | YGSSYGYFD | SENIYSY | TAK | QYGPPF |
| | SEQ ID | SEQ ID | SEQ ID NO: 149 | SEQ ID NO: 150 | SEQ ID | SEQ ID |
| | NO: 147 | NO: 148 | | | NO: 129 | NO: 151 |
| 34 | GYTFTSY | PSDS | YSKYYAMD | SQSIVHSYGNTY | KVS | GSHVPY |
| | SEQ ID | SEQ ID | SEQ ID NO: 153 | SEQ ID NO: 154 | SEQ ID | SEQ ID |
| | NO: 147 | NO: 152 | | | NO: 29 | NO: 155 |
| 35 | GYTFTSY | PSDS | GNYGENAMD | SQSIVHSYGNTY | KVS | GSHVPF |
| | SEQ ID | SEQ ID | SEQ ID NO: 156 | SEQ ID NO: 154 | SEQ ID | SEQ ID |
| | NO: 147 | NO: 152 | | | NO: 29 | NO: 157 |
| 36 | GFNIKDY | PEDG | GFA | SSSVSSSY | STS | WSSHPY |
| | SEQ ID | SEQ ID | SEQ ID NO: 160 | SEQ ID NO: 161 | SEQ ID | SEQ ID |
| | NO: 158 | NO: 159 | | | NO: 138 | NO: 162 |
| 37 | GYSFTSY | PSDS | GTGTLAYFA | SQSLVHSYGNTY | RVS | GTHVPY |
| | SEQ ID | SEQ ID | SEQ ID NO: 164 | SEQ ID NO: 165 | SEQ ID | SEQ ID |
| | NO: 163 | NO: 152 | | | NO: 142 | NO: 166 |
| 38 | GYTFRGY | AYGG | AGSSWYDFD | SQGISSW | AAS | GASFPI |
| | SEQ ID | SEQ ID | SEQ ID NO: 169 | SEQ ID NO: 170 | SEQ ID | SEQ ID |
| | NO: 167 | NO: 168 | | | NO: 44 | NO: 171 |

TABLE C4
CRDs for illustrative anti-SIRPα binding antibidies (Honegger)

| Ab | | | | | | VL - |
|---|---|---|---|---|---|---|
| Name | VH - CDR1 | VH - CDR2 | VH - CDR3 | VL - CDR1 | VL - CDR2 | CDR3 |
| 39 | ASGYTFTSYW | IYPGSGSTNHIEKFKSK | GYGSSYGYFD | ASENIYSY | TAKTLAEGVPSR | QYGPPF |
| | SEQ ID NO: 172 | SEQ ID NO: 173 | SEQ ID NO: 174 | SEQ ID NO: 175 | SEQ ID NO: 176 | SEQ ID |
| | | | | | | NO: 151 |
| 40 | ASGYTFTSYW | IDPSDSDTHYNQKFKDR | GYSKYYAMD | SSQSIVHSYGNTY | KVSNRFSGVPDR | GSHVPY |
| | SEQ ID NO: 172 | SEQ ID NO: 177 | SEQ ID NO: 178 | SEQ ID NO: 179 | SEQ ID NO: 180 | SEQ ID |
| | | | | | | NO: 155 |
| 41 | ASGYTFTSYW | IDPSDSDTHYNQKFKDK | YGNYGENAMD | SSQSIVHSYGNTY | KVSNRFSGVPDR | GSHVPF |
| | SEQ ID NO: 172 | SEQ ID NO: 181 | SEQ ID NO: 182 | SEQ ID NO: 179 | SEQ ID NO: 180 | SEQ ID |
| | | | | | | NO: 157 |
| 42 | ASGFNIKDYY | IDPEDGETKYAPKFQGK | GGFA | ASSSVSSSY | STSNLASGVPAR | WSSHPY |
| | SEQ ID NO: 183 | SEQ ID NO: 184 | SEQ ID NO: 185 | SEQ ID NO: 186 | SEQ ID NO: 187 | SEQ ID |
| | | | | | | NO: 162 |
| 43 | ASGYSFTSYW | IDPSDSDTHYSPSFQGH | GGTGTLAYFA | SSQSLVHSYGNTY | RVSNRFSGVPDR | GTHVPY |
| | SEQ ID NO: 188 | SEQ ID NO: 189 | SEQ ID NO: 190 | SEQ ID NO: 191 | SEQ ID NO: 192 | SEQ ID |
| | | | | | | NO: 166 |
| 44 | ASGYTFRGYG | ISAYGGETNYAQKLQGR | EAGSSWYDFD | ASQGISSW | AASNLQSGVPSR | GASFPI |
| | SEQ ID NO: 193 | SEQ ID NO: 194 | SEQ ID NO: 195 | SEQ ID NO: 196 | SEQ ID NO: 197 | SEQ ID |
| | | | | | | NO: 171 |

TABLE D
VH/VL for illustrative anti-SIRPα binding antibodies

| Ab Name | VH | VL |
|---|---|---|
| 45 | SEQ ID NO: 198 | SEQ ID NO: 199 |
| | QVQLVQSGAEVKKPGASVKVSCKASGYTFTSYWITWVKQAP | DIQMTQSPSSLSASVGDRVTITCRASENIYSYLAWYQQ |
| | GQGLEWIGDIYPGSGSTNHIEKFKSKATLTVDTSISTAYME | KPGKAPKLLIYTAKTLAEGVPSRFSGSGSGTDFTLTIS |
| | LSRLRSDDTAVYYCATGYGSSYGYFDYWGQGTLVTVSS | SLQPEDFATYYCQHQYGPPFTFGQGTKLEIK |
| 46 | SEQ ID NO: 200 | SEQ ID NO: 201 |
| | QVQLVQSGAEVKKPGASVKVSCKASGYTFTSYWMHWVRQAP | DIVMTQTPLSLSVTPGQPASISCRSSQSIVHSYGNTYL |
| | GQGLEWMGNIDPSDSDTHYNQKFKDRVTMTRDTSTSTVYME | EWYLQKPGQSPQLLIYKVSNRFSGVPDRFSGSGSGTDF |
| | LSSLRSEDTAVYYCARGYSKYYAMDYWGQGTLVTVSS | TLKISRVEAEDVGVYYCFQGSHVPYTFGQGTKLEIK |
| 47 | SEQ ID NO: 202 | SEQ ID NO: 203 |
| | QVKLQESGAELVRPGSSVKLSCKASGYTFTSYWMHWVKQRP | DILMTQTPLSLPVSLGDQASISCRSSQSIVHSYGNTYL |
| | IQGLEWIGNIDPSDSDTHYNQKFKDKATLTVDNSSSTAYMQ | EWYLQKPGQSPKLLIYKVSNRFSGVPDRFSGSGSGTDF |
| | LSSLTSEDSAVYYCASYGNYGENAMDYWGQGTSVTVSS | TLKISRVEAEDLGVYYCFQGSHVPFTFGSGTKLEIK |
| 48 | SEQ ID NO: 204 | SEQ ID NO: 205 |
| | EVQLQQSGAELVKPGASVKLSCTASGFNIKDYYIHWVKQRT | QIVLTQSPAIMSASPGEKVTLTCSASSSVSSSYLYWYQ |
| | EQGLEWIGRIDPEDGETKYAPKFQGKATITADTSSNTAYLQ | QKPGSSPKLWIYSTSNLASGVPARFSGSGSGTSYSLTI |
| | LNSLTSEDTAVYSCAKGGFAYWGQGTLVTVSA | SSMEAEDAASYFCHQWSSHPYTFGGGTKLEIK |
| 49 | SEQ ID NO: 206 | SEQ ID NO: 207 |
| | EVQLVQSGAEVKKPGESLRISCKASGYSFTSYWVHWVRQMP | DVVMTQSPLSLPVTLGQPASISCRSSQSLVHSYGNTYL |
| | GKGLEWMGNIDPSDSDTHYSPSFQGHVTLSVDKSISTAYLQ | YWFQQRPGQSPRLLIYRVSNRFSGVPDRFSGSGSGTDF |
| | LSSLKASDTAMYYCVRGGTGTLAYFAYWGQGTLVTVSS | TLKISRVEAEDVGVYYCFQGTHVPYTFGGGTKVEIK |
| 50 | SEQ ID NO: 208 | SEQ ID NO: 209 |
| | QVQLVQSGAEVKKPGASVKVSCKASGYTFRGYGISWVRQAP | DIQMTQSPSSVSASVGDRVTITCRASQGISSWLAWYQQ |
| | GQGLEWMGWISAYGGETNYAQKLQGRVTMTTDTSTSTAYME | KPGKAPKLLIYAASNLQSGVPSRFSGSGSGTDFTLTIS |
| | LRSLRSDDTAVYYCAREAGSSWYDFDLWGRGTLVTVSS | SLQPEDFATYYCQQGASFPITFGGGTKVEIK |

c. SIRPα-Fc Fusion Protein

In various embodiments, the agent that inhibits binding between CD47 and SIRPα CD47 is a SIRPα-Fc fusion protein or a “high affinity SIRPα reagent”, which includes SIRPα-derived polypeptides and analogs thereof. High affinity SIRPα reagents are described in international application WO2013109752A1, which is hereby specifically incorporated by reference. High affinity SIRPα reagents are variants of the native SIRPα protein. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

A high affinity SIRPα reagent comprises the portion of SIRPα that is sufficient to bind CD47 at a recognizable affinity, e.g., high affinity, which normally lies between the signal sequence and the transmembrane domain, or a fragment thereof that retains the binding activity. The high affinity SIRPα reagent will usually comprise at least the d1 domain of SIRPα with modified amino acid residues to increase affinity. In some embodiments, a SIRPα variant is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with lg molecules. The amino acid changes that provide for increased affinity are localized in the d1 domain, and thus high affinity SIRPα reagents comprise a d1 domain of human SIRPα, with at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e., dimer, trimer, tetramer, etc.

Illustrative SIRPα-Fc fusion proteins of use include ALX-148 (a.k.a., evorpacept, described in WO2013109752), timdarpacept, TTI-621 or TTI-622 (described in WO2014094122), SIRPα-F8, JY002-M2G1(N297A), JMT601 (CPO107), SS002M91, SIRPalpha-lgG4-Fc-Fc, and hCD172a(SIRPα)-Fc-LIGHT.

3. Agent that Inhibits Binding Between Vascular Endothelial Growth Factor A (VEGFA) and One or More VEGFA Cognate Receptors

The methods described herein involve co-administration of an agent that inhibits binding between CD47 and SIRPα and an agent that inhibits binding between vascular endothelial growth factor A (VEGFA) and one or more VEGFA cognate receptors (VEGFA/VEFGR inhibiting agent). VEGFA has the alternative acronyms VEGF, MVCD1, VPF and is assigned NCBI Gene ID: 7422; Uniprot P15692. VEGFA cognate receptors include VEGFR1 (fms related receptor tyrosine kinase 1 (FLT1; NCBI Gene ID: 2321) and VEGFR2 (kinase insert domain receptor (KDR; a.k.a., CD309, FLK1; NCBI Gene ID 3791).

Illustrative examples of agents that inhibit binding between VEGFA and one or more VEGFA cognate receptors include without limitation an antibody that binds to VEGFA, an antibody that binds VEGFR2, a VEGFA-Fc fusion protein, and a small molecule inhibitor.

Exemplary antibodies that bind to VEGFA of use in the present methods include without limitation bevacizumab, ranibizumab, brolucizumab, ivonescimab, sevacizumab and faricimab. In some embodiments, that antibody that binds to VEGFA is bevacizumab.

Exemplary antibodies that bind to VEGFR2 of use in the present methods include without limitation ramucirumab.

Exemplary VEGFA-Fc fusion protein of use in the present methods include without limitation conbercept and aflibercept.

Exemplary small molecule inhibitors of VEGFA and one or more VEGFA cognate receptors of use in the present methods include without limitation surufatinib, catequentinib hydrochloride, fruquintinib, tivozanib, regorafenib, axitinib, vandetanib, chiauranib, pazopanib hydrochloride, sunitinib malate and pegaptanib octasodium.

In various embodiments, the antibody that binds to VEGFA comprises a VH-CDR1, a VH-CDR2, a VH-CDR3, a VL-CDR1, a VL-CDR2 and a VL-CDR3 comprising the following amino acid sequences (according to Kabat), respectively:
SEQ ID NOs: 258, 259, 260, 261, 262 and 263;
SEQ ID NOs: 264, 259, 265, 261, 262 and 263;
SEQ ID NOs: 266, 267, 268, 269, 270 and 271; or
SEQ ID NOs: 272, 273, 274, 275, 276 and 277.

In various embodiments, the antibody that binds to VEGFA comprises a VH-CDR1, a VH-CDR2, a VH-CDR3, a VL-CDR1, a VL-CDR2 and a VL-CDR3 comprising the following amino acid sequences (according to IMGT), respectively:
SEQ ID NOs: 278, 279, 280, 281, 282 and 263;
SEQ ID NOs: 283, 279, 284, 281, 282 and 263;
SEQ ID NOs: 285, 286, 287, 288, 289 and 271; or
SEQ ID NOs: 290, 291, 292, 293, 294 and 277.

In various embodiments, the antibody that binds to VEGFA comprises a VH-CDR1, a VH-CDR2, a VH-CDR3, a VL-CDR1, a VL-CDR2 and a VL-CDR3 comprising the following amino acid sequences (according to Chothia), respectively:
SEQ ID NOs: 295, 296, 297, 298, 282 and 299;
SEQ ID NOs: 300, 296, 301, 298, 282 and 299;
SEQ ID NOs: 302, 303, 304, 305, 289 and 306; or
SEQ ID NOs: 307, 308, 309, 310, 294 and 311.

In various embodiments, the antibody that binds to VEGFA comprises a VH-CDR1, a VH-CDR2, a VH-CDR3, a VL-CDR1, a VL-CDR2 and a VL-CDR3 comprising the following amino acid sequences (according to Honegger), respectively:
SEQ ID NOs: 312, 313, 314, 315, 316 and 299;
SEQ ID NOs: 317, 313, 318, 315, 316 and 299;
SEQ ID NOs: 319, 320, 321, 322, 323 and 306; or
SEQ ID NOs: 324, 325, 326, 327, 328 and 311.

In various embodiments, the antibody that binds to VEGFA comprises a VH-CDR1, a VH-CDR2, a VH-CDR3, a VL-CDR1, a VL-CDR2 and a VL-CDR3 comprising the following amino acid sequences, respectively:
SEQ ID NOs: 258, 259, 260, 261, 262 and 263 (according to Kabat);
SEQ ID NOs: 278, 279, 280, 281, 282 and 263 (according to IMGT);
SEQ ID NOs: 295, 296, 297, 298, 282 and 299 (according to Chothia); or
SEQ ID NOs: 312, 313, 314, 315, 316 and 299 (according to Honegger).

In various embodiments, the antibody that binds to VEGFA comprises a VH-CDR1, a VH-CDR2, a VH-CDR3, a VL-CDR1, a VL-CDR2 and a VL-CDR3 comprising the following amino acid sequences, respectively:
SEQ ID NOs: 264, 259, 265, 261, 262 and 263 (according to Kabat);
SEQ ID NOs: 283, 279, 284, 281, 282 and 263 (according to IMGT);
SEQ ID NOs: 300, 296, 301, 298, 282 and 299 (according to Chothia); or
SEQ ID NOs: 317, 313, 318, 315, 316 and 299 (according to Honegger).

In various embodiments, the antibody that binds to VEGFA comprises a VH-CDR1, a VH-CDR2, a VH-CDR3, a VL-CDR1, a VL-CDR2 and a VL-CDR3 comprising the following amino acid sequences, respectively:
SEQ ID NOs: 266, 267, 268, 269, 270 and 271 (according to Kabat);
SEQ ID NOs: 285, 286, 287, 288, 289 and 271 (according to IMGT);
SEQ ID NOs: 302, 303, 304, 305, 289 and 306 (according to Chothia); or
SEQ ID NOs: 319, 320, 321, 322, 323 and 306 (according to Honegger).

In various embodiments, the antibody that binds to VEGFA comprises a VH-CDR1, a VH-CDR2, a VH-CDR3, a VL-CDR1, a VL-CDR2 and a VL-CDR3 comprising the following amino acid sequences, respectively:
SEQ ID NOs: 272, 273, 274, 275, 276 and 277 (according to Kabat);
SEQ ID NOs: 290, 291, 292, 293, 294 and 277 (according to IMGT);
SEQ ID NOs: 307, 308, 309, 310, 294 and 311 (according to Chothia); or
SEQ ID NOs: 324, 325, 326, 327, 328 and 311 (according to Honegger).

In various embodiments, the antibody that binds to VEGFA comprises a VH and a VL comprising the amino acid sequences set forth, respectively, or comprise amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth, respectively, in:
SEQ ID NOs: 329, 330, 331, 332, 333, 334, 335, 336, 337 or 338. Sequence identity can be determined according to the BLAST algorithm (blast.ncbi.nlm.nih.gov/Blast.cgi), using default settings.

Amino acid sequences of CDRs and variable regions (VH/VL) of illustrative anti-VEGFA antibodies that can be used in the present methods are described in Tables E1, E2, E3, E4 and F.

TABLE E1
CDRs for illustrative anti-VEGFA binding antibodies (Kabat)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 61 | NYGMN | WINTYTGEPTYAADFKR | YPHYYGSSHWYFDV | SASQDISNYLN | FTSSLHS | QQYSTVPWT |
| | SEQ ID | SEQ ID NO: 259 | SEQ ID NO: 260 | SEQ ID NO: 261 | SEQ ID | SEQ ID |
| | NO: 258 | | | | NO: 262 | NO: 263 |
| 62 | HYGMN | WINTYTGEPTYAADFKR | YPYYYGTSHWYFDV | SASQDISNYLN | FTSSLHS | QQYSTVPWT |
| | SEQ ID | SEQ ID NO: 259 | SEQ ID NO: 265 | SEQ ID NO: 261 | SEQ ID | SEQ ID |
| | NO: 264 | | | | NO: 262 | NO: 263 |
| 63 | NNDVMC | CIMTTDVVTEYANWAKS | DSVGSPLMSFDL | QASQSIYNNNELS | RASTLAS | GGYKSYSNDGNG |
| | SEQ ID | SEQ ID NO: 267 | SEQ ID NO: 268 | SEQ ID NO: 269 | SEQ ID | SEQ ID |
| | NO: 266 | | | | NO: 270 | NO: 271 |
| 64 | DYYYMT | FIDPDDDPYYATWAKG | GDHNSGWGLDI | QASEIIHSWLA | LASTLAS | QNVYLASTNGAN |
| | SEQ ID | SEQ ID NO: 273 | SEQ ID NO: 274 | SEQ ID NO: 275 | SEQ ID | SEQ ID |
| | NO: 272 | | | | NO: 276 | NO: 277 |

TABLE E2
CDRs for illustrative anti-VEGFA binding antibodies (IMGT)

| Ab | | | | | VL - | |
|---|---|---|---|---|---|---|
| Name | VH - CDR1 | VH - CDR2 | VH - CDR3 | VL - CDR1 | CDR2 | VL - CDR3 |
| 65 | GYTFTNYG | INTYTGEP | AKYPHYYGSSHWYFDV | QDISNY | FTS | QQYSTVPWT |
| | SEQ ID NO: 278 | SEQ ID | SEQ ID NO: 280 | SEQ ID NO: 281 | SEQ ID | SEQ ID NO: 263 |
| | | NO: 279 | | | NO: 282 | |
| 66 | GYDFTHYG | INTYTGEP | AKYPYYYGTSHWYFDV | QDISNY | FTS | QQYSTVPWT |
| | SEQ ID NO: 283 | SEQ ID | SEQ ID NO: 284 | SEQ ID NO: 281 | SEQ ID | SEQ ID NO: 263 |
| | | NO: 279 | | | NO: 282 | |
| 67 | GFSFSNNDV | IMTTDVVT | ARDSVGSPLMSFDL | QSIYNNNE | RAS | GGYKSYSNDGNG |
| | SEQ ID NO: 285 | SEQ ID | SEQ ID NO: 287 | SEQ ID NO: 288 | SEQ ID | SEQ ID NO: 271 |
| | | NO: 286 | | | NO: 289 | |
| 68 | GFSLTDYYY | IDPDDDP | AGGDHNSGWGLDI | EIIHSW | LAS | QNVYLASTNGAN |
| | SEQ ID NO: 290 | SEQ ID | SEQ ID NO: 292 | SEQ ID NO: 293 | SEQ ID | SEQ ID NO: 277 |
| | | NO: 291 | | | NO: 294 | |

TABLE E3
CDRs for illustrative anti-VEGFA binding antibodis (Chothia)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 69 | GYTFTNY | TYTG | PHYYGSSHWYFD | SQDISNY | FTS | YSTVPW |
| | SEQ ID NO: 295 | SEQ ID | SEQ ID NO: 297 | SEQ ID NO: 298 | SEQ ID | SEQ ID NO: 299 |
| | | NO: 296 | | | NO: 282 | |
| 70 | GYDFTHY | TYTG | PYYYGTSHWYFD | SQDISNY | FTS | YSTVPW |
| | SEQ ID NO: 300 | SEQ ID | SEQ ID NO: 301 | SEQ ID NO: 298 | SEQ ID | SEQ ID NO: 299 |
| | | NO: 296 | | | NO: 282 | |
| 71 | GFSFSNND | TTDV | SVGSPLMSFD | SQSIYNNNE | RAS | YKSYSNDGN |
| | SEQ ID NO: 302 | SEQ ID | SEQ ID NO: 304 | SEQ ID NO: 305 | SEQ ID | SEQ ID NO: 306 |
| | | NO: 303 | | | NO: 289 | |
| 72 | GFSLTDYY | PDD | DHNSGWGLD | SEITHSW | LAS | VYLASTNGA |
| | SEQ ID NO: 307 | SEQ ID | SEQ ID NO: 309 | SEQ ID NO: 310 | SEQ ID | SEQ ID NO: 311 |
| | | NO: 308 | | | NO: 294 | |

TABLE E4
CDRs for illustrative anti-VEGFA binding antibodies (Honegger)

| Ab | | | | | | |
|---|---|---|---|---|---|---|
| Name | VH-CDR1 | VH-CDR2 | VH-CDR3 | VL-CDR1 | VL - CDR2 | VL-CDR3 |
| 73 | ASGYTFTNYG | INTYTGEPTYAADFKRR | YPHYYGSSHWYFD | ASQDISNY | FTSSLHSGVPSR | YSTVPW |
| | SEQ ID | SEQ ID NO: 313 | SEQ ID NO: 314 | SEQ ID | SEQ ID NO: 316 | SEQ ID |
| | NO: 312 | | | NO: 315 | | NO: 299 |
| 74 | ASGYDFTHYG | INTYTGEPTYAADFKRR | YPYYYGTSHWYFD | ASQDISNY | FTSSLHSGVPSR | YSTVPW |
| | SEQ ID | SEQ ID NO: 313 | SEQ ID NO: 318 | SEQ ID | SEQ ID NO: 316 | SEQ ID |
| | NO: 317 | | | NO: 315 | | NO: 299 |
| 75 | ASGFSFSNNDV | IMTTDVVTEYANWAKSR | DSVGSPLMSFD | ASQSIYNNNE | RASTLASGVPSR | YKSYSNDGN |
| | SEQ ID | SEQ ID NO: 320 | SEQ ID NO: 321 | SEQ ID | SEQ ID NO: 323 | SEQ ID |
| | NO: 319 | | | NO: 322 | | NO: 306 |
| 76 | ASGFSLTDYYY | IDPDDDPYYATWAKGR | GDHNSGWGLD | ASEIIHSW | LASTLASGVPSR | VYLASTNGA |
| | SEQ ID | SEQ ID NO: 325 | SEQ ID NO: 326 | SEQ ID | SEQ ID NO: 328 | SEQ ID |
| | NO: 324 | | | NO: 327 | | NO: 311 |

TABLE F
VH/VL for illustrative anti-VEGFA binding antibodies

| Ab Name | VH | VL |
|---|---|---|
| 81 | SEQ ID NO: 329 | SEQ ID NO: 330 |
| | EVQLVESGGGLVQPGGSLRLSCAASGYTFTNYGMNWVRQAP | DIQMTQSPSSLSASVGDRVTITCSASQDISNYLNWYQQ |
| | GKGLEWVGWINTYTGEPTYAADFKRRFTFSLDTSKSTAYLQ | KPGKAPKVLIYFTSSLHSGVPSRFSGSGSGTDFTLTIS |
| | MNSLRAEDTAVYYCAKYPHYYGSSHWYFDVWGQGTLVTVSS | SLQPEDFATYYCQQYSTVPWTFGQGTKVEIKR |
| 82 | SEQ ID NO: 331 | SEQ ID NO: 332 |
| | EVQLVESGGGLVQPGGSLRLSCAASGYDFTHYGMNWVRQAP | DIQLTQSPSSLSASVGDRVTITCSASQDISNYLNWYQQ |
| | GKGLEWVGWINTYTGEPTYAADFKRRFTFSLDTSKSTAYLQ | KPGKAPKVLIYFTSSLHSGVPSRFSGSGSGTDFTLTIS |
| | MNSLRAEDTAVYYCAKYPYYYGTSHWYFDVWGQGTLVTVSS | SLQPEDFATYYCQQYSTVPWTFGQGTKVEIKR |
| 83 | SEQ ID NO: 333 | SEQ ID NO: 334 |
| | EVQLVESGGGLVQPGGSLRLSCAASGYDFTHYGMNVRQAPG | DIQLTQSPSSLSASVGDRVTITCSASQDISNYLNWYQQ |
| | KGLEWVGWINTYTGEPTYAADFKRRFTFSLDTSKSTAYLQM | KPGKAPKVLIYFTSSLHSGVPSRFSGSGSGTDFTLTIS |
| | NSLRAEDTAVYYCAKYPYYYGTSHWYFDVWGQGTLYTVSS | SLQPEDFATYYCQQYSTVPWTFGQGTKVEIKR |
| 84 | SEQ ID NO: 335 | SEQ ID NO: 336 |
| | EVQLVESGGGLVKPGGSLRLSCAASGFSFSNNDVMCWVRQA | DIQMTQSPSSLSASVGDRVTINCQASQSIYNNNELSWY |
| | PGKGLEWIGCIMTTDVVTEYANWAKSRFTVSRDSAKNSVYL | QQKPGKPPKLLIYRASTLASGVPSRFSGSGSGTDFTLT |
| | QMNSLRAEDTAVYFCARDSVGSPLMSFDLWGPGTLVTVSS | ISSLQPEDVATYYCGGYKSYSNDGNGFGGGTKVEIK |
| 85 | SEQ ID NO: 337 | SEQ ID NO: 338 |
| | EVQLVESGGGLVQPGGSLRLSCTASGFSLTDYYYMTWVRQA | EIVMTQSPSTLSASVGDRVIITCQASEIIHSWLAWYQQ |
| | PGKGLEWVGFIDPDDDPYYATWAKGRFTISRDNSKNTLYLQ | KPGKAPKLLIYLASTLASGVPSRFSGSGSGAEFTLTIS |
| | MNSLRAEDTAVYYCAGGDHNSGWGLDIWGQGTLVTVSS | SLQPDDFATYYCQNVYLASTNGANFGQGTKLTVL |

4. Additional Combination Agents

Additional agents, such as small molecules, antibodies, adoptive cellular therapies and chimeric antigen receptor T cells (CAR-T), checkpoint inhibitors, and vaccines, that are appropriate for treating hematological malignancies can be administered in combination with the agent that inhibits binding between CD47 and SIRPα (e.g., magrolimab); and the VEGFA/VEGFR inhibiting agent (e.g., bevacizumab), as described herein. Additional immunotherapeutic agents for hematological malignancies are described in Dong, et al, J Life Sci (Westlake Village). 2019 June; 1(1): 46-52; and Cuesta-Mateos, et al, Front. Immunol. 8:1936. doi: 10.3389/fimmu.2017.01936, each of which are hereby incorporated by reference in their entireties for all purposes.

In various embodiments, the agent that inhibits binding between CD47 and SIRPα (e.g., magrolimab); and the VEGFA/VEGFR inhibiting agent (e.g., bevacizumab), as described herein, is further combined with one or more additional therapeutic agents, e.g., an inhibitory immune checkpoint blocker or inhibitor, a stimulatory immune checkpoint stimulator, agonist or activator, a chemotherapeutic agent, an anti-cancer agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (mono- and multi-specific antibodies and fragments thereof in any format (e.g., including without limitation DARTs®, Duobodies®, BiTEs®, BiKEs, TriKEs, XmAbs®, TandAbs®, scFvs, Fabs, Fab derivatives), bi-specific antibodies, non-immunoglobulin antibody mimetics (e.g., including without limitation adnectins, affibody molecules, affilins, affimers, affitins, alphabodies, anticalins, peptide aptamers, armadillo repeat proteins (ARMs), atrimers, avimers, designed ankyrin repeat proteins (DARPins®), fynomers, knottins, Kunitz domain peptides, monobodies, and nanoCLAMPs), antibody-drug conjugates (ADC), antibody-peptide conjugate), an oncolytic virus, a gene modifier or editor, a cell comprising a chimeric antigen receptor (CAR), e.g., including a T cell immunotherapeutic agent, an NK-cell immunotherapeutic agent, or a macrophage immunotherapeutic agent, a cell comprising an engineered T-cell receptor (TCR-T), or any combination thereof.

In various embodiments, the agent that inhibits binding between CD47 and SIRPα (e.g., magrolimab); and the VEGFA/VEGFR inhibiting agent (e.g., bevacizumab), as described herein, is further combined with one or more additional therapeutic agents including, without limitation, an inhibitor, agonist, antagonist, ligand, modulator, stimulator, blocker, activator or suppressor of a target (e.g., polypeptide or polynucleotide) including without limitation: Abelson murine leukemia viral oncogene homolog 1 gene (ABL, such as ABL1), Acetyl-CoA carboxylase (such as ACC1/2), activated CDC kinase (ACK, such as ACK1), Adenosine deaminase, adenosine receptor (such as A2BR, A2aR, A3aR), Adenylate cyclase, ADP ribosyl cyclase-1, adrenocorticotropic hormone receptor (ACTH), Aerolysin, AKT1 gene, Alk-5 protein kinase, Alkaline phosphatase, Alpha 1 adrenoceptor, Alpha 2 adrenoceptor, Alpha-ketoglutarate dehydrogenase (KGDH), Aminopeptidase N, AMP activated protein kinase, anaplastic lymphoma kinase (ALK, such as ALK1), Androgen receptor, Angiopoietin (such as ligand-1, ligand-2), Angiotensinogen (AGT) gene, murine thymoma viral oncogene homolog 1 (AKT) protein kinase (such as AKT1, AKT2, AKT3), apolipoprotein A-I (APOA1) gene, Apoptosis inducing factor, apoptosis protein (such as 1, 2), apoptosis signal-regulating kinase (ASK, such as ASK1), Arginase (I), Arginine deiminase, Aromatase, Asteroid homolog 1 (ASTE1) gene, ataxia telangiectasia and Rad 3 related (ATR) serine/threonine protein kinase, Aurora protein kinase (such as 1, 2), Axl tyrosine kinase receptor, 4-1BB ligand (CD137L), Baculoviral IAP repeat containing 5 (BIRC5) gene, Basigin, B-cell lymphoma 2 (BCL2) gene, Bcl2 binding component 3, Bcl2 protein, BCL2L11 gene, BCR (breakpoint cluster region) protein and gene, Beta adrenoceptor, Beta-catenin, B-lymphocyte antigen CD19, B-lymphocyte antigen CD20, B-lymphocyte cell adhesion molecule, B-lymphocyte stimulator ligand, Bone morphogenetic protein-10 ligand, Bone morphogenetic protein-9 ligand modulator, Brachyury protein, Bradykinin receptor, B-Raf proto-oncogene (BRAF), Brc-Abl tyrosine kinase, Bromodomain and external domain (BET) bromodomain containing protein (such as BRD2, BRD3, BRD4), Bruton's tyrosine kinase (BTK), Calmodulin, calmodulin-dependent protein kinase (CaMK, such as CAMKII), Cancer testis antigen 2, Cancer testis antigen NY-ESO-1, cancer/testis antigen 1B (CTAG1) gene, Cannabinoid receptor (such as CB1, CB2), Carbonic anhydrase, casein kinase (CK, such as CKI, CKII), Caspase (such as caspase-3, caspase-7, Caspase-9), caspase 8 apoptosis-related cysteine peptidase CASP8-FADD-like regulator, Caspase recruitment domain protein-15, Cathepsin G, CCR5 gene, CDK-activating kinase (CAK), Checkpoint kinase (such as CHK1, CHK2), chemokine (C-C motif) receptor (such as CCR2, CCR4, CCR5, CCR8), chemokine (C-X-C motif) receptor (such as CXCR1, CXCR2, CXCR3 and CXCR4), Chemokine CC21 ligand, Cholecystokinin CCK2 receptor, Chorionic gonadotropin, c-Kit (tyrosine-protein kinase Kit or CD117), CISH (Cytokine-inducible SH2-containing protein), Claudin (such as 6, 18), cluster of differentiation (CD) such as CD4, CD27, CD29, CD30, CD33, CD37, CD40, CD40 ligand receptor, CD40 ligand, CD40LG gene, CD44, CD45, CD47, CD49b, CD51, CD52, CD55, CD58, CD66e (CEACAM6), CD70 gene, CD74, CD79, CD79b, CD79B gene, CD80, CD95, CD99, CD117, CD122, CDw123, CD134, CDw137, CD158a, CD158b1, CD158b2, CD223, CD276 antigen; clusterin (CLU) gene, Clusterin, c-Met (hepatocyte growth factor receptor (HGFR)), Complement C3, Connective tissue growth factor, COP9 signalosome subunit 5, CSF-1 (colony-stimulating factor 1 receptor), CSF2 gene, CTLA-4 (cytotoxic T-lymphocyte protein 4) receptor, C-type lectin domain protein 9A (CLEC9A), Cyclin D1, Cyclin G1, cyclin-dependent kinases (CDK, such as CDK1, CDK12, CDK1B, CDK2-9), cyclooxygenase (such as COX1, COX2), CYP2B1 gene, Cysteine palmitoyltransferase porcupine, Cytochrome P450 11B2, Cytochrome P450 17, cytochrome P450 17A1, Cytochrome P450 2D6, cytochrome P450 3A4, Cytochrome P450 reductase, cytokine signalling-1, cytokine signalling-3, Cytoplasmic isocitrate dehydrogenase, Cytosine deaminase, cytosine DNA methyltransferase, cytotoxic T-lymphocyte protein-4, DDR2 gene, DEAD-box helicase 6 (DDX6), Death receptor 5 (DR5, TRAILR2), Death receptor 4 (DR4, TRAILR1), Delta-like protein ligand (such as 3, 4), Deoxyribonuclease, Deubiquitinating enzymes (DUBs), Dickkopf-1 ligand, dihydrofolate reductase (DHFR), Dihydropyrimidine dehydrogenase, Dipeptidyl peptidase IV, discoidin domain receptor (DDR, such as DDR1), Diacylglycerol kinase zeta (DGKZ), DNA binding protein (such as HU-beta), DNA dependent protein kinase, DNA gyrase, DNA methyltransferase, DNA polymerase (such as alpha), DNA primase, dUTP pyrophosphatase, L-dopachrome tautomerase, E3 ubiquitin-protein ligase (such as RNF128, CBL-B), echinoderm microtubule like protein 4, EGFR tyrosine kinase receptor, Elastase, Elongation factor 1 alpha 2, Elongation factor 2, Endoglin, Endonuclease, endoplasmic reticulum aminopeptidase (ERAP, such as ERAP 1, ERAP2), Endoplasmin, Endosialin, Endostatin, endothelin (such as ET-A, ET-B), Enhancer of zeste homolog 2 (EZH2), Ephrin (EPH) tyrosine kinase (such as Epha3, Ephb4), Ephrin B2 ligand, epidermal growth factor, epidermal growth factor receptors (EGFR), epidermal growth factor receptor (EGFR) gene, Epigen, Epithelial cell adhesion molecule (EpCAM), Erb-b2 (v-erb-b2 avian erythroblastic leukemia viral oncogene homolog 2) tyrosine kinase receptor, Erb-b3 tyrosine kinase receptor, Erb-b4 tyrosine kinase receptor, E-selectin, Estradiol 17 beta dehydrogenase, Estrogen receptor (such as alpha, beta), Estrogen related receptor, Eukaryotic translation initiation factor 5A (EIF5A) gene, Exportin 1, Extracellular signal related kinase (such as 1, 2), Extracellular signal-regulated kinases (ERK), Hypoxia-inducible factor prolyl hydroxylase (HIF-PH or EGLN), Factor (such as Xa, VIIa), farnesoid x receptor (FXR), Fas ligand, Fatty acid synthase (FASN), Ferritin, FGF-2 ligand, FGF-5 ligand, fibroblast growth factor (FGF, such as FGF1, FGF2, FGF4), Fibronectin, focal adhesion kinase (FAK, such as FAK2), folate hydrolase prostate-specific membrane antigen 1 (FOLH1), Folate receptor (such as alpha), Folate, Folate transporter 1, FYN tyrosine kinase, paired basic amino acid cleaving enzyme (FURIN), Beta-glucuronidase, Galactosyltransferase, Galectin-3, Ganglioside GD2, Glucocorticoid, glucocorticoid-induced TNFR-related protein GITR receptor, Glutamate carboxypeptidase II, glutaminase, Glutathione S-transferase P, glycogen synthase kinase (GSK, such as 3-beta), Glypican 3 (GPC3), gonadotropin-releasing hormone (GNRH), Granulocyte macrophage colony stimulating factor (GM-CSF) receptor, Granulocyte-colony stimulating factor (GCSF) ligand, growth factor receptor-bound protein 2 (GRB2), Grp78 (78 kDa glucose-regulated protein) calcium binding protein, molecular chaperone groEL2 gene, Heme oxygenase 1 (HO1), Heme oxygenase 2 (H02), Heat shock protein (such as 27, 70, 90 alpha, beta), Heat shock protein gene, Heat stable enterotoxin receptor, Hedgehog protein, Heparanase, Hepatocyte growth factor, HERV-H LTR associating protein 2, Hexose kinase, Histamine H2 receptor, Histone methyltransferase (DOT1L), histone deacetylase (HDAC, such as 1, 2, 3, 6, 10, 11), Histone H1, Histone H3, HLA class I antigen (A-2 alpha), HLA class II antigen, HLA class I antigen alpha G (HLA-G), Non-classical HLA, Homeobox protein NANOG, HSPB1 gene, Human leukocyte antigen (HLA), Human papillomavirus (such as E6, E7) protein, Hyaluronic acid, Hyaluronidase, Hypoxia inducible factor-1 alpha (HIFlu), Imprinted Maternally Expressed Transcript (H19) gene, mitogen-activated protein kinase 1 (MAP4K1), tyrosine-protein kinase HCK, I-Kappa-B kinase (IKK, such as IKKbe), IL-1 alpha, IL-1 beta, IL-12, IL-12 gene, IL-15, IL-17, IL-2 gene, IL-2 receptor alpha subunit, IL-2, IL-3 receptor, IL-4, IL-6, IL-7, IL-8, immunoglobulin (such as G, G1, G2, K, M), Immunoglobulin Fc receptor, Immunoglobulin gamma Fc receptor (such as I, III, IIIA), indoleamine 2,3-dioxygenase (IDO, such as IDO1 and IDO2), indoleamine pyrrole 2,3-dioxygenase 1 inhibitor, insulin receptor, Insulin-like growth factor (such as 1, 2), Integrin alpha-4/beta-1, integrin alpha-4/beta-7, Integrin alpha-5/beta-1, Integrin alpha-V/beta-3, Integrin alpha-V/beta-5, Integrin alpha-V/beta-6, Intercellular adhesion molecule 1 (ICAM-1), interferon (such as alpha, alpha 2, beta, gamma), Interferon inducible protein absent in melanoma 2 (AIM2), interferon type I receptor, Interleukin 1 ligand, Interleukin 13 receptor alpha 2, interleukin 2 ligand, interleukin-1 receptor-associated kinase 4 (IRAK4), Interleukin-2, Interleukin-29 ligand, Interleukin 35 (IL-35), isocitrate dehydrogenase (such as IDH1, IDH2), Janus kinase (JAK, such as JAK1, JAK2), Jun N terminal kinase, kallikrein-related peptidase 3 (KLK3) gene, Killer cell Ig like receptor, Kinase insert domain receptor (KDR), Kinesin-like protein KIF11, Kirsten rat sarcoma viral oncogene homolog (KRAS) gene, Kisspeptin (KiSS-1) receptor, KIT gene, v-kit Hardy-Zuckerman 4 feline sarcoma viral oncogene homolog (KIT) tyrosine kinase, lactoferrin, Lanosterol-14 demethylase, LDL receptor related protein-1, Leukocyte immunoglobulin-like receptor subfamily B member 1 (ILT2), Leukocyte immunoglobulin-like receptor subfamily B member 2 (ILT4), Leukotriene A4 hydrolase, Listeriolysin, L-Selectin, Luteinizing hormone receptor, Lyase, lymphocyte activation gene 3 protein (LAG-3), Lymphocyte antigen 75, Lymphocyte function antigen-3 receptor, lymphocyte-specific protein tyrosine kinase (LCK), Lymphotactin, Lyn (Lck/Yes novel) tyrosine kinase, lysine demethylases (such as KDM1, KDM2, KDM4, KDM5, KDM6, A/B/C/D), Lysophosphatidate-1 receptor, lysosomal-associated membrane protein family (LAMP) gene, Lysyl oxidase homolog 2, lysyl oxidase protein (LOX), 5-Lipoxygenase (5-LOX), Hematopoietic Progenitor Kinase 1 (HPK1), Hepatocyte growth factor receptor (MET) gene, macrophage colony-stimulating factor (MCSF) ligand, Macrophage migration inhibitory fact, MAGEC1 gene, MAGEC2 gene, Major vault protein, MAPK-activated protein kinase (such as MK2), Mas-related G-protein coupled receptor, matrix metalloprotease (MMP, such as MMP2, MMP9), Mel-1 differentiation protein, Mdm2 p53-binding protein, Mdm4 protein, Melan-A (MART-1) melanoma antigen, Melanocyte protein Pmel 17, melanocyte stimulating hormone ligand, melanoma antigen family A3 (MAGEA3) gene, Melanoma associated antigen (such as 1, 2, 3, 6), Membrane copper amine oxidase, Mesothelin, MET tyrosine kinase, Metabotropic glutamate receptor 1, Metalloreductase STEAPI (six transmembrane epithelial antigen of the prostate 1), Metastin, methionine aminopeptidase-2, Methyltransferase, Mitochondrial 3 ketoacyl CoA thiolase, mitogen-activate protein kinase (MAPK), mitogen-activated protein kinase (MEK, such as MEK1, MEK2), mTOR (mechanistic target of rapamycin (serine/threonine kinase), mTOR complex (such as 1,2), mucin (such as 1, 5A, 16), mut T homolog (MTH, such as MTH1), Myc proto-oncogene protein, myeloid cell leukemia 1 (MCL1) gene, myristoylated alanine-rich protein kinase C substrate (MARCKS) protein, NAD ADP ribosyltransferase, natriuretic peptide receptor C, Neural cell adhesion molecule 1, Neurokinin 1 (NKI) receptor, Neurokinin receptor, Neuropilin 2, NF kappa B activating protein, NIMA-related kinase 9 (NEK9), Nitric oxide synthase, NK cell receptor, NK3 receptor, NKG2 A B activating NK receptor, NLRP3 (NACHT LRR PYD domain protein 3) modulators, Noradrenaline transporter, Notch (such as Notch-2 receptor, Notch-3 receptor, Notch-4 receptor), Nuclear erythroid 2-related factor 2, Nuclear Factor (NF) kappa B, Nucleolin, Nucleophosmin, nucleophosmin-anaplastic lymphoma kinase (NPM-ALK), 2 oxoglutarate dehydrogenase, 2,5-oligoadenylate synthetase, O-methylguanine DNA methyltransferase, Opioid receptor (such as delta), Ornithine decarboxylase, Orotate phosphoribosyltransferase, orphan nuclear hormone receptor NR4A1, Osteocalcin, Osteoclast differentiation factor, Osteopontin, OX-40 (tumor necrosis factor receptor superfamily member 4 TNFRSF4, or CD134) receptor, P3 protein, p38 kinase, p38 MAP kinase, p53 tumor suppressor protein, Parathyroid hormone ligand, peroxisome proliferator-activated receptors (PPAR, such as alpha, delta, gamma), P-Glycoprotein (such as 1), phosphatase and tensin homolog (PTEN), phosphatidylinositol 3-kinase (PI3K), phosphoinositide-3 kinase (PI3K such as alpha, delta, gamma), phosphorylase kinase (PK), PKN3 gene, placenta growth factor, platelet-derived growth factor (PDGF, such as alpha, beta), Platelet-derived growth factor (PDGF, such as alpha, beta), Pleiotropic drug resistance transporter, Plexin B1, PLK1 gene, polo-like kinase (PLK), Polo-like kinase 1, Poly (ADP-ribose) polymerase (PARP, such as PARP1, PARP2 and PARP3, PARP7, and mono-PARPs), Preferentially expressed antigen in melanoma (PRAME) gene, Prenyl-binding protein (PrPB), Probable transcription factor PML, Progesterone receptor, Programmed cell death 1 (PD-1), Programmed cell death ligand 1 inhibitor (PD-L1), Prosaposin (PSAP) gene, Prostanoid receptor (EP4), Prostaglandin E2 synthase, prostate specific antigen, Prostatic acid phosphatase, proteasome, Protein E7, Protein farnesyltransferase, protein kinase (PK, such as A, B, C), protein tyrosine kinase, Protein tyrosine phosphatase beta, Proto-oncogene serine/threonine-protein kinase (PIM, such as PIM-1, PIM-2, PIM-3), P-Selectin, Purine nucleoside phosphorylase, purinergic receptor P2X ligand gated ion channel 7 (P2X7), Pyruvate dehydrogenase (PDH), Pyruvate dehydrogenase kinase, Pyruvate kinase (PYK), 5-Alpha-reductase, Raf protein kinase (such as 1, B), RAFI gene, Ras gene, Ras GTPase, RET gene, Ret tyrosine kinase receptor, retinoblastoma associated protein, retinoic acid receptor (such as gamma), Retinoid X receptor, Rheb (Ras homolog enriched in brain) GTPase, Rho (Ras homolog) associated protein kinase 2, ribonuclease, Ribonucleotide reductase (such as M2 subunit), Ribosomal protein S6 kinase, RNA polymerase (such as I, II), Ron (Recepteur d'Origine Nantais) tyrosine kinase, ROS1 (ROS proto-oncogene 1, receptor tyrosine kinase) gene, RosI tyrosine kinase, Runt-related transcription factor 3, Gamma-secretase, S100 calcium binding protein A9, Sarco endoplasmic calcium ATPase, Second mitochondria-derived activator of caspases (SMAC) protein, Secreted frizzled related protein-2, Secreted phospholipase A2, Semaphorin-4D, Serine protease, serine/threonine kinase (STK), serine/threonine-protein kinase (TBK, such as TBK1), signal transduction and transcription (STAT, such as STAT-1, STAT-3, STAT-5), Signaling lymphocytic activation molecule (SLAM) family member 7, six-transmembrane epithelial antigen of the prostate (STEAP) gene, SL cytokine ligand, smoothened (SMO) receptor, Sodium iodide cotransporter, Sodium phosphate cotransporter 2B, Somatostatin receptor (such as 1, 2, 3, 4, 5), Sonic hedgehog protein, Son of sevenless (SOS), Specific protein 1 (Sp1) transcription factor, Sphingomyelin synthase, Sphingosine kinase (such as 1, 2), Sphingosine-1-phosphate receptor-1, spleen tyrosine kinase (SYK), SRC gene, Src tyrosine kinase, Stabilin-1 (STAB1), STAT3 gene, Steroid sulfatase, Stimulator of interferon genes (STING) receptor, stimulator of interferon genes protein, Stromal cell-derived factor 1 ligand, SUMO (small ubiquitin-like modifier), Superoxide dismutase, Suppressor of cytokine signaling modulators (SOCS), Survivin protein, Synapsin 3, Syndecan-1, Synuclein alpha, T cell surface glycoprotein CD28, tank-binding kinase (TBK), TATA box-binding protein-associated factor RNA polymerase I subunit B (TAF1B) gene, T-cell CD3 glycoprotein zeta chain, T-cell differentiation antigen CD6, T-cell immunoglobulin and mucin-domain containing-3 (TIM-3), T-cell surface glycoprotein CD8, Tec protein tyrosine kinase, Tek tyrosine kinase receptor, telomerase, Telomerase reverse transcriptase (TERT) gene, Tenascin, Three prime repair exonuclease 1 (TREX1), Three prime repair exonuclease 2 (TREX2), Thrombopoietin receptor, Thymidine kinase, Thymidine phosphorylase, Thymidylate synthase, Thymosin (such as alpha 1), Thyroid hormone receptor, Thyroid stimulating hormone receptor, Tissue factor, TNF related apoptosis inducing ligand, TNFR1 associated death domain protein, TNF-related apoptosis-inducing ligand (TRAIL) receptor, TNFSF11 gene, TNFSF9 gene, Toll-like receptor (TLR such as 1-13), topoisomerase (such as I, II, III), Transcription factor, Transferase, transferrin (TF), transforming growth factor alpha (TGFα), transforming growth factor beta (TGFB) and isoforms thereof, TGF beta 2 ligand, Transforming growth factor TGF-β receptor kinase, Transglutaminase, Translocation associated protein, Transmembrane glycoprotein NMB, Trop-2 calcium signal transducer, trophoblast glycoprotein (TPBG) gene, Trophoblast glycoprotein, Tropomyosin receptor kinase (Trk) receptor (such as TrkA, TrkB, TrkC), tryptophan 2,3-dioxygenase (TDO), Tryptophan 5-hydroxylase, Tubulin, Tumor necrosis factor (TNF, such as alpha, beta), Tumor necrosis factor 13C receptor, tumor progression locus 2 (TPL2), Tumor protein 53 (TP53) gene, Tumor suppressor candidate 2 (TUSC2) gene, Tumor specific neoantigens, Tyrosinase, Tyrosine hydroxylase, tyrosine kinase (TK), Tyrosine kinase receptor, Tyrosine kinase with immunoglobulin-like and EGF-like domains (TIE) receptor, Tyrosine protein kinase ABL1 inhibitor, Ubiquitin, Ubiquitin carboxyl hydrolase isozyme L5, Ubiquitin thioesterase-14, Ubiquitin-conjugating enzyme E21 (UBE2I, UBC9), Ubiquitin-specific-processing protease 7 (USP7), Urease, Urokinase plasminogen activator, Uteroglobin, Vanilloid VR1, Vascular cell adhesion protein 1, vascular endothelial growth factor receptor (VEGFR), V-domain Ig suppressor of T-cell activation (VISTA), VEGF-1 receptor, VEGF-2 receptor, VEGF-3 receptor, VEGF-A, VEGF-B, Vimentin, Vitamin D3 receptor, Proto-oncogene tyrosine-protein kinase, Mer (Mer tyrosine kinase receptor modulators), YAP (Yes-associated protein modulators)es, Wee-1 protein kinase, Werner Syndrome RecQ Like Helicase (WRN), Wilms' tumor antigen 1, Wilms' tumor protein, WW domain containing transcription regulator protein 1 (TAZ), X-linked inhibitor of apoptosis protein, Zinc finger protein transcription factor or any combination thereof.

In various embodiments, the agent that inhibits binding between CD47 and SIRPα (e.g., magrolimab); and the VEGFA/VEGFR inhibiting agent (e.g., bevacizumab), as described herein, is combined with one or more additional therapeutic agents that may be categorized by their mechanism of action into, for example, the following groups: anti-metabolites/anti-cancer agents, such as pyrimidine analogs floxuridine, capecitabine, cytarabine, CPX-351 (liposomal cytarabine, daunorubicin), and TAS-118; Alpha 1 adrenoceptor/Alpha 2 adrenoceptor antagonists, such as phenoxybenzamine hydrochloride (injectable, pheochromocytoma); Androgen receptor antagonists, such as nilutamide; anti-cadherin antibodies, such as HKT-288; anti-leucine-rich repeat containing 15 (LRRC15) antibodies, such as ABBV-085. ARGX-110; angiotensin receptor blockers, nitric oxide donors; antisense oligonucleotides, such as AEG35156, IONIS-KRAS-2.5Rx, EZN-3042, RX-0201, IONIS-AR-2.5Rx, BP-100 (prexigebersen), IONIS-STAT3-2.5Rx; anti-angiopoietin (ANG)-2 antibodies, such as MEDI3617, and LY3127804; anti-ANG-1/ANG-2 antibodies, such as AMG-780; anti-CSF1R antibodies, such as emactuzumab, LY3022855, AMG-820, FPA-008 (cabiralizumab); anti-endoglin antibodies, such as TRC105 (carotuximab); anti-ERBB antibodies, such as CDX-3379, HLX-02, seribantumab; anti-HER2 antibodies, such as HERCEPTIN® (trastuzumab), trastuzumab biosimimar, margetuximab, MEDI4276, BAT-8001, Pertuzumab (Perjeta), RG6264, ZW25 (a bispecific HER2-directed antibody targeting the extracellular domains 2 and 4; Cancer Discov. 2019 January; 9(1):8; PMID: 30504239); anti-HLA-DR antibodies, such as IMMU-114; anti-IL-3 antibodies, such as JNJ-56022473; anti-TNF receptor superfamily member 18 (TNFRSF18, GITR; NCBI Gene ID: 8784) antibodies, such as MK-4166, MEDI1873, FPA-154, INCAGN-1876, TRX-518, BMS-986156, MK-1248, GWN-323; and those described, e.g., in Intl. Patent Publ. Nos. WO 2017/096179, WO 2017/096276, WO 2017/096189; and WO 2018/089628; anti-EphA3 antibodies, such as KB-004; anti-CD37 antibodies, such as otlertuzumab (TRU-016); anti-FGFR-3 antibodies, such as LY3076226, B-701; anti-FGFR-2 antibodies, such as GAL-F2; anti-C5 antibodies, such as ALXN-1210; anti-EpCAM antibodies, such as VB4-845; anti-CEA antibodies, such as RG-7813; anti-Carcinoembryonic-antigen-related-cell-adhesion-molecule-6 (CEACAM6, CD66C) antibodies, such as BAY-1834942, NEO-201 (CEACAM 5/6); anti-GD2 antibodies, such as APN-301; anti-interleukin-17 (IL-17) antibodies, such as CJM-112; anti-interleukin-1 beta antibodies, such as canakinumab (ACZ885), VPM087; anti-carbonic anhydrase 9 (CA9, CAIX) antibodies, such as TX-250; anti-Mucin 1 (MUC1) antibodies, such as gatipotuzumab, Mab-AR-20.5; anti-KMA antibodies, such as MDX-1097; anti-CD55 antibodies, such as PAT-SCi; anti-c-Met antibodies, such as ABBV-399; anti-PSMA antibodies, such as ATL-101; anti-CD100 antibodies, such as VX-15; anti-EPHA3 antibodies, such as fibatuzumab; anti-APRIL antibodies, such as BION-1301; anti-fibroblast activation protein (FAP)/IL-2R antibodies, such as RG7461; anti-fibroblast activation protein (FAP)/TRAIL-R2 antibodies, such as RG7386; anti-fucosyl-GM1 antibodies, such as BMS-986012; anti-IL-8 (Interleukin-8) antibodies, such as HuMax-Inflam; anti-myostatin inhibitors, such as landogrozumab; anti-delta-like protein ligand 3 (DDL3) antibodies, such as rovalpituzumab tesirine; anti-DLL4 (delta like ligand 4) antibodies, such as demcizumab; anti-clusterin antibodies, such as AB-16B5; anti-Ephrin-A4 (EFNA4) antibodies, such as PF-06647263; anti-mesothelin antibodies, such as BMS-986148, Anti-MSLN-MMAE; anti-sodium phosphate cotransporter 2B (NaP2B) antibodies, such as lifastuzumab; anti-TGFβ antibodies, such as SAR439459; anti-transforming growth factor-beta (TGF-beta) antibodies, such as ABBV-151, LY3022859, NIS793, XOMA 089; purine analogs, folate antagonists (such as pralatrexate), cladribine, pentostatin, fludarabine and related inhibitors; antiproliferative/antimitotic agents including natural products, such as vinca alkaloids (vinblastine, vincristine) and microtubule disruptors such as taxane (paclitaxel, docetaxel), vinblastin, nocodazole, epothilones, vinorelbine (NAVELBINE®), and epipodophyllotoxins (etoposide, teniposide); DNA damaging agents, such as actinomycin, amsacrine, busulfan, carboplatin, chlorambucil, cisplatin, cyclophosphamide (CYTOXAN®), dactinomycin, daunorubicin, doxorubicin, DEBDOX, epirubicin, iphosphamide, melphalan, merchlorethamine, mitomycin C, mitoxantrone, nitrosourea, procarbazine, taxol, Taxotere, teniposide, etoposide, and triethylenethiophosphoramide; DNA-hypomethylating agents, such as guadecitabine (SGI-110), oral decitabine and cedazuridine (ASTX727); antibiotics such as dactinomycin, daunorubicin, doxorubicin, idarubicin, anthracyclines, mitoxantrone, bleomycins, plicamycin (mithramycin); enzymes such as L-asparaginase which systemically metabolizes L-asparagine and deprives cells which do not have the capacity to synthesize their own asparagine; DNAi oligonucleotides targeting Bcl-2, such as PNT2258; agents that activate or reactivate latent human immunodeficiency virus (HIV), such as panobinostat and romidepsin; asparaginase stimulators, such as crisantaspase (Erwinase®) and GRASPA (ERY-001, ERY-ASP), calaspargase pegol, pegaspargase; pan-Trk, ROS1 and ALK inhibitors, such as entrectinib, TPX-0005; anaplastic lymphoma kinase (ALK) inhibitors, such as alectinib, ceritinib, alecensa (RG7853), ALUNBRIG® (brigatinib); antiproliferative/antimitotic alkylating agents, such as nitrogen mustard cyclophosphamide and analogs (e.g., melphalan, chlorambucil, hexamethylmelamine, thiotepa), alkyl nitrosoureas (e.g., carmustine) and analogs, streptozocin, and triazenes (e.g., dacarbazine); antiproliferative/antimitotic antimetabolites, such as folic acid analogs (methotrexate); platinum coordination complexes (e.g., cisplatin, oxiloplatinim, and carboplatin), procarbazine, hydroxyurea, mitotane, and aminoglutethimide; hormones, hormone analogs (e.g., estrogen, tamoxifen, goserelin, bicalutamide, and nilutamide), and aromatase inhibitors (e.g., letrozole and anastrozole); antiplatelet agents; anticoagulants such as heparin, synthetic heparin salts, and other inhibitors of thrombin; fibrinolytic agents such as tissue plasminogen activator, streptokinase, urokinase, aspirin, dipyridamole, ticlopidine, and clopidogrel; antimigratory agents; antisecretory agents (e.g., breveldin); immunosuppressives, such as tacrolimus, sirolimus, azathioprine, and mycophenolate; growth factor inhibitors, and vascular endothelial growth factor inhibitors; fibroblast growth factor inhibitors, such as FPA14; AMP activated protein kinase stimulators, such as metformin hydrochloride; ADP ribosyl cyclase-1 inhibitors, such as daratumumab (DARZALEX®); Caspase recruitment domain protein-15 stimulators, such as mifamurtide (liposomal); CCR5 chemokine antagonists, such as MK-7690 (vicriviroc); CDC7 protein kinase inhibitors, such as TAK-931; Cholesterol side-chain cleavage enzyme inhibitors, such as ODM-209; Dihydropyrimidine dehydrogenase/Orotate phosphoribosyltransferase inhibitors, such as Cefesone (tegafur+gimeracil+oteracil potassium); DNA polymerase/Ribonucleotide reductase inhibitors, such as clofarabine; DNA interference oligonucleotides, such as PNT2258, AZD-9150; Estrogen receptor modulators, such as bazedoxifene; Estrogen receptor agonists/Progesterone receptor antagonists, such as TRI-CYCLEN LO (norethindrone+ethinyl estradiol); HLA class I antigen A-2 alpha modulators, such as FH-MCVA2TCR; HLA class I antigen A-2 alpha/MART-1 melanoma antigen modulators, such as MART-1 F5 TCR engineered PBMC; Human Granulocyte Colony Stimulating Factors, such as PF-06881894; GNRH receptor agonists, such as leuprorelin acetate, leuprorelin acetate sustained release depot (ATRIGEL), triptorelin pamoate, goserelin acetate; GNRH receptor antagonists, such as elagolix, relugolix, degarelix; Endoplasmin modulators, such as anlotinib; H+K+ ATPase inhibitors, such as omeprazole, esomeprazole; ICAM-1/CD55 modulators, such as cavatak (V-937); IL-15/IL-12 modulators, such as SAR441000; Interleukin 23A inhibitors, such as guselkumab; Lysine specific histone demethylase 1 inhibitors, such as CC-90011; IL-12 Mrna, such as MEDI1191; RIG-I modulators, such as RGT-100; NOD2 modulators, such as SB-9200, and IR-103; Progesterone receptor agonists, such as levonorgestrel; Protein cereblon modulators, such as CC-92480, CC-90009; Protein cereblon modulators/DNA binding protein Ikaros inhibitors/Zinc finger binding protein Aiolos inhibitors, such as iberdomide; Retinoid X receptor modulators, such as alitretinoin, bexarotene (oral formulation); RIP-1 kinase inhibitors, such as GSK-3145095; selective oestrogen receptor degraders, such as AZD9833; SUMO inhibitors, such as TAK-981; Thrombopoietin receptor agonists, such as eltrombopag; Thyroid hormone receptor agonists, such as levothyroxine sodium; TNF agonists, such as tasonermin; Tyrosine phosphatase substrate 1 inhibitors, such as CC-95251; HER2 inhibitors, such as neratinib, tucatinib (ONT-380); EGFR/ErbB2/Ephb4 inhibitors, such as tesevatinib; EGFR/HER2 inhibitors, such as TAK-788; EGFR family tyrosine kinase receptor inhibitors, such as DZD-9008; EGFR/ErbB-2 inhibitors, such as varlitinib; mutant selective EGFR inhibitors, such as PF-06747775, EGF816 (nazartinib), ASP8273, ACEA-0010, BI-1482694; epha2 inhibitors, such as MM-310; polycomb protein (EED) inhibitors, such as MAK683; DHFR inhibitor/Folate transporter 1 modulator/Folate receptor antagonist, such as pralatrexate; DHFR/GAR transformylase/Thymidylate synthase/Transferase inhibitors, such as pemetrexed disodium; p38 MAP kinase inhibitors, such as ralimetinib; PRMT inhibitors, such as MS203, PF-06939999, GSK3368715, GSK3326595; Sphingosine kinase 2 (SK2) inhibitors, such as opaganib; Nuclear erythroid 2-related factor 2 stimulators, such as omaveloxolone (RTA-408); Tropomyosin receptor kinase (TRK) inhibitors, such as LOXO-195, ONO-7579; Mucin 1 inhibitors, such as GO-203-2C; MARCKS protein inhibitors, such as BIO-11006; Folate antagonists, such as arfolitixorin; Galectin-3 inhibitors, such as GR-MD-02; Phosphorylated P68 inhibitors, such as RX-5902; CD95/TNF modulators, such as ofranergene obadenovec; pan-PIM kinase inhibitors, such as INCB-053914; IL-12 gene stimulators, such as EGEN-001, tavokinogene telseplasmid; Heat shock protein HSP90 inhibitors, such as TAS-116, PEN-866; VEGF/HGF antagonists, such as MP-0250; VEGF ligand inhibitors, such as bevacizumab biosimilar; VEGF receptor antagonists/VEGF ligand inhibitors, such as ramucirumab; VEGF-1/VEGF-2/VEGF-3 receptor antagonists; such as fruquintinib; VEGF-1/VEGF-2 receptor modulators, such as HLA-A2402/HLA-A0201 restricted epitope peptide vaccine; Placenta growth factor ligand inhibitor/VEGF-A ligand inhibitor, such as aflibercept; SYK tyrosine kinase/JAK tyrosine kinase inhibitors, such as ASN-002; Trk tyrosine kinase receptor inhibitors, such as larotrectinib sulfate; JAK3/JAK1/TBK1 kinase inhibitors, such as CS-12912; IL-24 antagonist, such as AD-IL24; NLRP3 (NACHT LRR PYD domain protein 3) modulators, such as BMS-986299; RIG-I agonists, such as RGT-100; Aerolysin stimulators, such as topsalysin; P-Glycoprotein 1 inhibitors, such as HM-30181A; CSF-1 antagonists, such as ARRY-382, BLZ-945; CCR8 inhibitors, such as JTX-1811, I-309, SB-649701, HG-1013, RAP-310; anti-Mesothelin antibodies, such as SEL-403; Thymidine kinase stimulators, such as aglatimagene besadenovec; Polo-like kinase 1 inhibitors, such as PCM-075, onvansertib; NAE inhibitors, such as pevonedistat (MLN-4924); Trop-2 inhibitors, such as sacituzumab govitecan (TRODELVY®), TAS-4464; Pleiotropic pathway modulators, such as avadomide (CC-122); Amyloid protein binding protein-1 inhibitors/Ubiquitin ligase modulators; FoxMI inhibitors, such as thiostrepton; UBA1 inhibitors, such as TAK-243; Src tyrosine kinase inhibitors, such as VAL-201; VDAC/HK inhibitors, such as VDA-1102; Elf4a inhibitors, such as rohinitib, eFT226; TP53 gene stimulators, such as ad-p53; Retinoic acid receptor agonists, such as tretinoin; Retinoic acid receptor alpha (RARu) inhibitors, such as SY-1425; SIRT3 inhibitors, such as YC8-02; Stromal cell-derived factor 1 ligand inhibitors, such as olaptesed pegol (NOX-A12); IL-4 receptor modulators, such as MDNA-55; Arginase-I stimulators, such as pegzilarginase; Topoisomerase I inhibitors, such as irinotecan hydrochloride, Onivyde; Topoisomerase I inhibitor/hypoxia inducible factor-1 alpha inhibitors, such as PEG-SN38 (firtecan pegol); Hypoxia inducible factor-1 alpha inhibitors, such as PT-2977, PT-2385; CD122 (IL-2 receptor) agonists, such as proleukin (aldesleukin, IL-2); pegylated IL-2 (e.g., NKTR-214); modified variants of IL-2 (e.g., THOR-707); TLR7/TLR8 agonist, such as NKTR-262; TLR7 agonists, such as DS-0509, GS-9620, LHC-165, TMX-101 (imiquimod); p53 tumor suppressor protein stimulators such as kevetrin; Mdm4/Mdm2 p53-binding protein inhibitors, such as ALRN-6924; kinesin spindle protein (KSP) inhibitors, such as filanesib (ARRY-520); CD80-Fc fusion protein inhibitors, such as FPT-155; Menin and mixed lineage leukemia (MLL) inhibitors such as KO-539; Liver x receptor agonists, such as RGX-104; IL-10 agonists, such as Pegilodecakin (AM-0010); VEGFR/PDGFR inhibitors, such as vorolanib; IRAK4 inhibitors, such as CA-4948; anti-TLR-2 antibodies, such as OPN-305; Calmodulin modulators, such as CBP-501.

Glucocorticoid receptor antagonists, such as relacorilant (CORT-125134); Second mitochondria-derived activator of caspases (SMAC) protein inhibitors, such as BI-891065; Lactoferrin modulators, such as LTX-315; KIT proto-oncogene, receptor tyrosine kinase (KIT) inhibitors, such as PLX-9486; platelet derived growth factor receptor alpha (PDGFRA)/KIT proto-oncogene, receptor tyrosine kinase (KIT) mutant-specific antagonists/inhibitors such as BLU-285, DCC-2618; Exportin 1 inhibitors, such as eltanexor; CHST15 gene inhibitors, such as STNM-01; Somatostatin receptor antagonist, such as OPS-201; CEBPA gene stimulators, such as MTL-501; DKK3 gene modulators, such as MTG-201; Chemokine (CXCR1/CXCR2) inhibitors, such as SX-682; p70s6k inhibitors, such as MSC2363318A; methionine aminopeptidase 2 (MetAP2) inhibitors, such as M8891, APL-1202; arginine N-methyltransferase 5 inhibitors, such as GSK-3326595; CD71 modulators, such as CX-2029 (ABBV-2029); ATM (ataxia telangiectasia) inhibitors, such as AZD0156, AZD1390; CHK1 inhibitors, such as GDC-0575, LY2606368 (prexasertib), SRA737, RG7741 (CHK1/2); CXCR4 antagonists, such as BL-8040, LY2510924, burixafor (TG-0054), X4P-002, X4P-001-IO, Plerixafor; EXH2 inhibitors, such as GSK2816126; KDM1 inhibitors, such as ORY-1001, IMG-7289, INCB-59872, GSK-2879552; CXCR2 antagonists, such as AZD-5069; DNA dependent protein kinase inhibitors, such as MSC2490484A (nedisertib), VX-984, AsiDNA (DT-01); protein kinase C (PKC) inhibitors, such as LXS-196, sotrastaurin; selective estrogen receptor downregulators (SERD), such as fulvestrant (Faslodex®), RG6046, RG6047, RG6171, elacestrant (RAD-1901), SAR439859 and AZD9496; selective estrogen receptor covalent antagonists (SERCAs), such as H3B-6545; selective androgen receptor modulator (SARM), such as GTX-024, darolutamide; transforming growth factor-beta (TGF-beta) kinase antagonists, such as galunisertib, LY3200882; TGF-beta inhibitors described in WO 2019/103203; TGF beta receptor 1 inhibitors, such as PF-06952229; bispecific antibodies, such as ABT-165 (DLL4/VEGF), MM-141 (IGF-1/ErbB3), MM-111 (Erb2/Erb3), JNJ-64052781 (CD19/CD3), PRS-343 (CD-137/HER2), AFM26 (BCMA/CD16A), JNJ-61186372 (EGFR/cMET), AMG-211 (CEA/CD3), RG7802 (CEA/CD3), ERY-974 (CD3/GPC3) vancizumab (angiopoietins/VEGF), PF-06671008 (Cadherins/CD3), AFM-13 (CD16/CD30), APV0436 (CD123/CD3), flotetuzumab (CD123/CD3), REGN-1979 (CD20/CD3), MCLA-117 (CD3/CLEC12A), MCLA-128 (HER2/HER3), JNJ-0819, JNJ-7564 (CD3/heme), AMG-757 (DLL3-CD3), MGD-013 (PD-1/LAG-3), FS-118 (LAG-3/PD-L1) MGD-019 (PD-1/CTLA-4), KN-046 (PD-1/CTLA-4), MEDI-5752 (CTLA-4/PD-1), RO-7121661 (PD-1/TIM-3), XmAb-20717 (PD-1/CTLA-4), AK-104 (CTLA-4/PD-1), AMG-420 (BCMA/CD3), BI-836880 (VEFG/ANG2), JNJ-63709178 (CD123/CD3), MGD-007 (CD3/gpA33), MGD-009 (CD3/B7H3), AGEN1223, IMCgp100 (CD3/gp100), AGEN-1423, ATOR-1015 (CTLA-4/OX40), LY-3415244 (TIM-3/PDL1), INHIBRX-105 (4-1BB/PDL1), faricimab (VEGF-A/ANG-2), FAP-4-IBBL (4-1BB/FAP), XmAb-13676 (CD3/CD20), TAK-252 (PD-1/OX40L), TG-1801 (CD19/CD47), XmAb-18087 (SSTR2/CD3), catumaxomab (CD3/EpCAM), SAR-156597 (IL4/IL13), EMB-01 (EGFR/cMET), REGN-4018 (MUC16/CD3), REGN-1979 (CD20/CD3), RG-7828 (CD20/CD3), CC-93269 (CD3/BCMA), REGN-5458 (CD3/BCMA), navicixizumab (DLL4/VEGF), GRB-1302 (CD3/Erbb2), vanucizumab (VEGF-A/ANG-2), GRB-1342 (CD38/CD3), GEM-333 (CD3/CD33), IMM-0306 (CD47/CD20), RG6076, MEDI5752 (PD-1/CTLA-4), LY3164530 (MET/EGFR); Alpha-ketoglutarate dehydrogenase (KGDH) inhibitors, such as CPI-613; XPO1 inhibitors, such as selinexor (KPT-330); Isocitrate dehydrogenase 2 (IDH2) inhibitors, such as enasidenib (AG-221); IDH1 inhibitors such as AG-120, and AG-881 (IDH1 and IDH2), IDH-305, BAY-1436032; IDH1 gene inhibitors, such as ivosidenib; interleukin-3 receptor (IL-3R) modulators, such as SL-401; Arginine deiminase stimulators, such as pegargiminase (ADI-PEG-20); claudin-18 inhibitors, such as claudiximab; β-catenin inhibitors, such as CWP-291; chemokine receptor 2 (CCR) inhibitors, such as PF-04136309, CCX-872, BMS-813160 (CCR2/CCR5); thymidylate synthase inhibitors, such as ONX-0801; ALK/ROS1 inhibtors, such as lorlatinib; tankyrase inhibitors, such as G007-LK; triggering receptor expressed on myeloid cells 1 (TREM1; NCBI Gene ID: 54210), such as PY159; triggering receptor expressed on myeloid cells 2 (TREM2; NCBI Gene ID: 54209), such as PY314; Mdm2 p53-binding protein inhibitors, such as CMG-097, HDM-201; c-PIM inhibitors, such as PIM447; sphingosine kinase-2 (SK2) inhibitors, such as Yeliva® (ABC294640); DNA polymerase inhibitors, such as sapacitabine; Cell cycle/Microtubule inhibitors, such as eribulin mesylate; c-MET inhibitors, such as AMG-337, savolitinib, tivantinib (ARQ-197), capmatinib, and tepotinib, ABT-700, AG213, AMG-208, JNJ-38877618 (OMO-1), merestinib, HQP-8361; c-Met/VEGFR inhibitors, such as BMS-817378, TAS-115; c-Met/RON inhibitors, such as BMS-777607; BCR/ABL inhibitors, such as rebastinib, asciminib, ponatinib (ICLUSIG®); MNK1/MNK2 inhibitors, such as eFT-508; Cytochrome P450 11B2/Cytochrome P450 17/AKT protein kinase inhibitors, such as LAE-201; Cytochrome P450 3A4 stimulators, such as mitotane; lysine-specific demethylase-1 (LSD1) inhibitors, such as CC-90011; CSF1R/KIT and FLT3 inhibitors, such as pexidartinib (PLX3397); Flt3 tyrosine kinase/Kit tyrosine kinase inhibitor and PDGF receptor antagonists, such as quizartinib dihydrochloride; kinase inhibitors, such as vandetanib; E selectin antagonists, such as GMI-1271; differentiation inducers, such as tretinoin; epidermal growth factor receptor (EGFR) inhibitors, such as osimertinib (AZD-9291), cetuximab; topoisomerase inhibitors, such as Adriamycin, doxorubicin, daunorubicin, dactinomycin, DaunoXome, Caelyx, eniposide, epirubicin, etoposide, idarubicin, irinotecan, mitoxantrone, pixantrone, sobuzoxane, topotecan, irinotecan, MM-398 (liposomal irinotecan), vosaroxin and GPX-150, aldoxorubicin, AR-67, mavelertinib, AST-2818, avitinib (ACEA-0010), irofulven (MGI-114); corticosteroids, such as cortisone, dexamethasone, hydrocortisone, methylprednisolone, prednisone, prednisolone; growth factor signal transduction kinase inhibitors; nucleoside analogs, such as DFP-10917; Axl inhibitors, such as BGB-324 (bemcentinib), SLC-0211; Axl/Flt3 inhibitors, such as gilteritinib; Inhibitors of bromodomain and extraterminal motif (BET) proteins, including ABBV-744, BRD2 (NCBI Gene ID: 6046), BRD3 (NCBI Gene ID: 8019), BRD4 (NCBI Gene ID: 23476), and bromodomain testis-specific protein (BRDT; NCBI Gene ID: 676), such as INCB-054329, INCB057643, TEN-010, AZD-5153, ABT-767, BMS-986158, CC-90010, GSK525762 (molibresib), NHWD-870, ODM-207, GSK-2820151, GSK-1210151A, ZBC246, ZBC260, ZEN3694, FT-1101, RG-6146, CC-90010, CC-95775, mivebresib, BI-894999, PLX-2853, PLX-51107, CPI-0610, GS-5829; PARP inhibitors, such as pamiparib, fuzuloparib, talazoparib tosylate, niraparib tosylate monohydrate, rucaparib camsylate, olaparib, veliparib, ABT-767, BGB-290, bendamustine hydrochloride; PARP/Tankyrase inhibitors such as 2X-121 (e-7499); IMP-4297, SC-10914, IDX-1197, HWH-340, CK-102, simmiparib; Proteasome inhibitors, such as ixazomib (NINLARO®), carfilzomib (Kyprolis®), marizomib, bortezomib; Glutaminase inhibitors, such as CB-839 (telaglenastat), bis-2-(5-phenylacetamido-1,3,4-thiadiazol-2-yl)ethyl sulfide (BPTES); mitochondrial complex I inhibitors, such as metformin, phenformin; vaccines, such as peptide vaccine TG-01 (RAS), GALE-301, GALE-302, nelipepimut-s, SurVaxM, DSP-7888, TPIV-200, PVX-410, VXL-100, DPX-E7, ISA-101, 6MHP, OSE-2101, galinpepimut-S, SVN53-67/M57-KLH, IMU-131, peptide subunit vaccine (acute lymphoblastic leukemia, University Children's Hospital Tuebingen); bacterial vector vaccines such as CRS-207/GVAX, axalimogene filolisbac (ADXS11-001); adenovirus vector vaccines such as nadofaragene firadenovec; autologous Gp96 vaccine; dendritic cells vaccines, such as CVactm, stapuldencel-T, eltrapuldencel-T, rocapuldencel-T (AGS-003), DCVAC, SL-701, BSK01TM, ADXS31-142, autologous dendritic cell vaccine (metastatic malignant melanoma, intradermal/intravenous, Universitatsklinikum Erlangen); oncolytic vaccines such as, talimogene laherparepvec, pexastimogene devacirepvec, GL-ONC1, MG1-MA3, parvovirus H-1, ProstAtak, enadenotucirev, MG1MA3, ASN-002 (TG-1042); therapeutic vaccines, such as CVAC-301, CMP-001, CreaVax-BC, PF-06753512, VBI-1901, TG-4010, ProscaVax™; tumor cell vaccines, such as Vigil® (IND-14205), Oncoquest-L vaccine; live attenuated, recombinant, serotype 1 poliovirus vaccine, such as PVS-RIPO; Adagloxad simolenin; MEDI-0457; DPV-001 a tumor-derived, autophagosome enriched cancer vaccine; RNA vaccines such as, CV-9209, LV-305; DNA vaccines, such as MEDI-0457, MVI-816, INO-5401; modified vaccinia virus Ankara vaccine expressing p53, such as MVA-p53; DPX-Survivac; BriaVax™; GI-6301; GI-6207; GI-4000; IO-103; Neoantigen peptide vaccines, such as AGEN-2017, GEN-010, NeoVax, RG-6180, GEN-009, PGV-001 (TLR-3 agonist), GRANITE-001, NEO-PV-01; Peptide vaccines that target heat shock proteins, such as PhosphoSynVax™; Vitespen (HSPPC-96-C), NANT Colorectal Cancer Vaccine containing aldoxorubicin, autologous tumor cell vaccine+systemic CpG-B+IFN-alpha (cancer), IO-120+IO-103 (PD-L1/PD-L2 vaccines), HB-201, HB-202, HB-301, TheraT®-based vaccines; TLR-3 agonist/interferon inducers, such as Poly-ICLC (NSC-301463); STAT-3 inhibitors, such as napabucasin (BBI-608); ATPase p97 inhibitors, such as CB-5083; smoothened (SMO) receptor inhibitors, such as Odomzo® (sonidegib, formerly LDE-225), LEQ506, vismodegib (GDC-0449), BMS-833923, glasdegib (PF-04449913), LY2940680, and itraconazole; interferon alpha ligand modulators, such as interferon alpha-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus)(Inmutag, Inter 2A), interferon alfa-2b follow-on biologic (Biosidus—Bioferon, Citopheron, Ganapar, Beijing Kawin Technology—Kaferon), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-n1 (Humoferon, SM-10500, Sumiferon); interferon gamma ligand modulators, such as interferon gamma (OH-6000, Ogamma 100); telomerase modulators, such as, tertomotide (GV-1001, HR-2802, Riavax) and imetelstat (GRN-163, JNJ-63935937); DNA methyltransferases inhibitors, such as temozolomide (CCRG-81045), decitabine, oral decitabine and cedazuridine (ASTX727), guadecitabine (5-110, SGI-110), KRX-0402, RX-3117, RRx-001, and azacytidine (CC-486); DNA gyrase inhibitors, such as pixantrone and sobuzoxane; DNA gyrase inhibitors/Topoisimerase II inhibitors, such as amrubicin; Bcl-2 family protein inhibitors, such as ABT-263, venetoclax (ABT-199), obatoclax mesylate, pelcitoclax, ABT-737, RG7601, and AT-101; Bcl-2/Bcl-XL inhibitors, such as navitoclax (ABT-263; RG-7433); Notch inhibitors, such as LY3039478 (crenigacestat), tarextumab (anti-Notch2/3), BMS-906024; hyaluronidase stimulators, such as PEGPH-20; Erbb2 tyrosine kinase receptor inhibitors/Hyaluronidase stimulators, such as Herceptin Hylecta; Wnt pathway inhibitors, such as SM-04755, PRI-724, WNT-974; gamma-secretase inhibitors, such as PF-03084014, MK-0752, RO-4929097; Grb-2 (growth factor receptor bound protein-2) inhibitors, such as BP1001; TRAIL pathway-inducing compounds, such as ONC201, ABBV-621; TRAIL modulators, such as SCB-313; Focal adhesion kinase inhibitors, such as VS-4718, defactinib, GSK2256098; hedgehog inhibitors, such as saridegib, sonidegib (LDE225), glasdegib; Aurora kinase inhibitors, such as alisertib (MLN-8237), and AZD-2811, AMG-900, barasertib, ENMD-2076; HSPB1 modulators (heat shock protein 27, HSP27), such as brivudine, apatorsen; ATR inhibitors, such as BAY-937, AZD6738, AZD6783, VX-803, VX-970 (berzosertib) and VX-970; Hsp90 inhibitors, such as AUY922, onalespib (AT13387), SNX-2112, SNX5422; murine double minute (mdm2) oncogene inhibitors, such as DS-3032b, RG7775, AMG-232, HDM201, and idasanutlin (RG7388); CD137 agonists, such as urelumab, utomilumab (PF-05082566), AGEN2373, ADG-106, BT-7480, QL1806; STING agonists, such as ADU-S100 (MIW-815), SB-11285, MK-1454, SR-8291, AdVCA0848, GSK-532, SYN-STING, MSA-1, SR-8291, GSK3745417; FGFR inhibitors, such as FGF-401, INCB-054828, BAY-1163877, AZD4547, JNJ-42756493, LY2874455, Debio-1347; fatty acid synthase (FASN) inhibitors, such as TVB-2640; CD44 binders, such as A6; protein phosphatease 2A (PP2A) inhibitors, such as LB-100; CYP17 inhibitors, such as seviteronel (VT-464), ASN-001, ODM-204, CFG920, abiraterone acetate; RXR agonists, such as IRX4204; hedgehog/smoothened (hh/Smo) antagonists, such as taladegib, patidegib, vismodegib; complement C3 modulators, such as Imprime PGG; IL-15 agonists, such as ALT-803, NKTR-255, interleukin-15/Fc fusion protein, AM-0015, NIZ-985, and hetIL-15; EZH2 (enhancer of zeste homolog 2) inhibitors, such as tazemetostat, CPI-1205, GSK-2816126, PF-06821497; oncolytic viruses, such as pelareorep, CG-0070, MV-NIS therapy, HSV-1716, DS-1647, VCN-01, ONCOS-102, TBI-1401, tasadenoturev (DNX-2401), vocimagene amiretrorepvec, RP-1, CVA21, Celyvir, LOAd-703, OBP-301, IMLYGIC®; DOT1L (histone methyltransferase) inhibitors, such as pinometostat (EPZ-5676); toxins such as Cholera toxin, ricin, Pseudomonas exotoxin, Bordetella pertussis adenylate cyclase toxin, diphtheria toxin, and caspase activators; DNA plasmids, such as BC-819; PLK inhibitors of PLK 1, 2, and 3, such as volasertib (PLK1); WEE1 inhibitors, such as AZD-1775 (adavosertib); Rho kinase (ROCK) inhibitors, such as AT13148, KD025; Inhibition of Apoptosis Protein (IAP) inhibitors, such as ASTX660, debio-1143, birinapant, APG-1387, LCL-161; RNA polymerase inhibitors, such has lurbinectedin (PM-1183), CX-5461; Tubulin inhibitors, such as PM-184, BAL-101553 (lisavanbulin), and OXI-4503, fluorapacin (AC-0001), plinabulin, vinflunine; Toll-like receptor 4 (TLR-4) agonists, such as G100, GSK1795091, and PEPA-10; Elongation factor 1 alpha 2 inhibitors, such as plitidepsin; Elongation factor 2 inhibitors/Interleukin-2 ligands/NAD ADP ribosyltransferase stimulators, such as denileukin diftitox; CD95 inhibitors, such as APG-101, APO-010, asunercept; WT1 inhibitors, such as DSP-7888; splicing factor 3B subunit1 (SF3B1) inhibitors, such as H3B-8800; retinoid Z receptor gamma (RORy) agonists, such as LYC-55716; and microbiome modulators, such as SER-401, EDP-1503, MRx-0518.

In various embodiments, the agent that inhibits binding between CD47 and SIRPα (e.g., magrolimab); and the VEGFA/VEGFR inhibiting agent (e.g., bevacizumab), as described herein, is further combined with one or more additional therapeutic agents comprising an inhibitor or antagonist of: myeloid cell leukemia sequence 1 (MCL1) apoptosis regulator (NCBI Gene ID: 4170); mitogen-activated protein kinase 1 (MAP4K1) (also called Hematopoietic Progenitor Kinase 1 (HPK1), NCBI Gene ID: 11184); diacylglycerol kinase alpha (DGKA, DAGK, DAGK1 or DGK-alpha; NCBI Gene ID: 1606); 5′-nucleotidase ecto (NT5E or CD73; NCBI Gene ID: 4907); ectonucleoside triphosphate diphosphohydrolase 1 (ENTPD1 or CD39; NCBI Gene ID: 593); transforming growth factor beta 1 (TGFB1 or TGFβ; NCBI Gene ID: 7040); heme oxygenase 1 (HMOX1, HO-1 or HO1; NCBI Gene ID: 3162); heme oxygenase 2 (HMOX2, HO-2 or H02; NCBI Gene ID: 3163); vascular endothelial growth factor A (VEGFA or VEGF; NCBI Gene ID: 7422); erb-b2 receptor tyrosine kinase 2 (ERBB2, HER2, HER2/neu or CD340; NCBI Gene ID: 2064), epidermal growth factor receptor (EGFR, ERBB, ERBB1 or HER1; NCBI Gene ID: 1956); ALK receptor tyrosine kinase (ALK, CD246; NCBI Gene ID: 238); poly(ADP-ribose) polymerase 1 (PARP1; NCBI Gene ID: 142); poly(ADP-ribose) polymerase 2 (PARP2; NCBI Gene ID: 10038); TCDD inducible poly(ADP-ribose) polymerase (TIPARP, PARP7; NCBI Gene ID: 25976); cyclin dependent kinase 4 (CDK4; NCBI Gene ID: 1019); cyclin dependent kinase 6 (CDK6; NCBI Gene ID: 1021); TNF receptor superfamily member 14 (TNFRSF14, HVEM, CD270; NCBI Gene ID: 8764); T cell immunoreceptor with Ig and ITIM domains (TIGIT; NCBI Gene ID: 201633); X-linked inhibitor of apoptosis (XIAP, BIRC4, IAP-3; NCBI Gene ID: 331); baculoviral IAP repeat containing 2 (BIRC2, cIAP1; NCBI Gene ID: 329); baculoviral IAP repeat containing 3 (BIRC3, cIAP2; NCBI Gene ID: 330); baculoviral IAP repeat containing 5 (BIRC5, surviving; NCBI Gene ID: 332); C-C motif chemokine receptor 2 (CCR2, CD192; NCBI Gene ID: 729230); C-C motif chemokine receptor 5 (CCR5, CD195; NCBI Gene ID: 1234); C-C motif chemokine receptor 8 (CCR8, CDw198; NCBI Gene ID: 1237); C-X-C motif chemokine receptor 2 (CXCR2, CD182; NCBI Gene ID: 3579); C-X-C motif chemokine receptor 3 (CXCR3, CD182, CD183; NCBI Gene ID: 2833); C-X-C motif chemokine receptor 4 (CXCR4, CD184; NCBI Gene ID: 7852); arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240) and/or soluble epoxide hydrolase 2 (EPHX2, SEH; NCBI Gene ID: 2053); a secreted phospholipase A2 (e.g., PLA2G1B (NCBI Gene ID: 5319); PLA2G7 (NCBI Gene ID: 7941), PLA2G3 (NCBI Gene ID: 50487), PLA2G2A (NCBI Gene ID: 5320); PLA2G4A (NCBI Gene ID: 5321); PLA2G12A (NCBI Gene ID: 81579); PLA2G12B (NCBI Gene ID: 84647); PLA2G10 (NCBI Gene ID: 8399); PLA2G5 (NCBI Gene ID: 5322); PLA2G2D (NCBI Gene ID: 26279); PLA2G15 (NCBI Gene ID: 23659)); indoleamine 2,3-dioxygenase 1 (IDO1; NCBI Gene ID: 3620); indoleamine 2,3-dioxygenase 2 (IDO2; NCBI Gene ID: 169355); hypoxia inducible factor 1 subunit alpha (HIF1A; NCBI Gene ID: 3091); angiopoietin 1 (ANGPT1; NCBI Gene ID: 284); Endothelial TEK tyrosine kinase (TIE-2, TEK, CD202B; NCBI Gene ID: 7010); Janus kinase 1 (JAKI; NCBI Gene ID: 3716); catenin beta 1 (CTNNB1; NCBI Gene ID: 1499); histone deacetylase 9 (HDAC9; NCBI Gene ID: 9734), and/or 5′-3′ exoribonuclease 1 (XRN1; NCBI Gene ID: 54464).

In various embodiments, the agent that inhibits binding between CD47 and SIRPα (e.g., magrolimab); and the VEGFA/VEGFR inhibiting agent (e.g., bevacizumab), as described herein, is further combined with an agonist of fms related receptor tyrosine kinase 3 (FLT3); FLK2; STK1; CD135; FLK-2; NCBI Gene ID: 2322). Examples of FLT3 agonists include, but are not limited to, CDX-301 and GS-3583. GS-3583 is described, e.g., in WO 2020/263830, hereby incorporated herein by reference in its entirety for all purposes.

In various embodiments, the agent that inhibits binding between CD47 and SIRPα (e.g., magrolimab); and the VEGFA/VEGFR inhibiting agent (e.g., bevacizumab), as described herein, is further combined with an anti-CD19 agent or antibody. Examples of anti-CD19 agents or antibodies that can be co-administered include without limitation: blinatumomab, tafasitamab, XmAb5574 (Xencor), AFM-11, inebilizumab, loncastuximab, MEDI 551 (Cellective Therapeutics); and MDX-1342 (Medarex).

In various embodiments, the agent that inhibits binding between CD47 and SIRPα (e.g., magrolimab); and the VEGFA/VEGFR inhibiting agent (e.g., bevacizumab), as described herein, is further combined with an anti-CD20 agent or antibody. Examples of anti-CD20 agents or antibodies that can be co-administered include without limitation: IGN-002, PF-05280586; Rituximab (Rituxan/Biogen Idec), Ofatumumab (Arzerra/Genmab), Obinutuzumab (Gazyva/Roche Glycart Biotech), Alemtuzumab, Veltuzumab, Veltuzumab, Ocrelizumab (Ocrevus/Biogen Idec; Genentech), Ocaratuzumab and Ublituximab, and LFB-R603 (LFB Biotech.; rEVO Biologics).

In various embodiments, the agent that inhibits binding between CD47 and SIRPα (e.g., magrolimab); and the VEGFA/VEGFR inhibiting agent (e.g., bevacizumab), as described herein, is further combined with an anti-CD22 agent or antibody. Examples of anti-CD22 agents or antibodies that can be co-administered include without limitation: Epratuzumab, AMG-412, IMMU-103 (Immunomedics).

In various embodiments, the agent that inhibits binding between CD47 and SIRPα (e.g., magrolimab); and the VEGFA/VEGFR inhibiting agent (e.g., bevacizumab), as described herein, is further combined with an anti-CD30 agent or antibody. Examples of anti-CD30 agents or antibodies that can be co-administered include without limitation: Brentuximab vedotin (Seattle Genetics).

In various embodiments, the agent that inhibits binding between CD47 and SIRPα (e.g., magrolimab); and the VEGFA/VEGFR inhibiting agent (e.g., bevacizumab), as described herein, is further combined with an anti-CD33 agent or antibody. Examples of anti-CD33 agents or antibodies that can be co-administered include without limitation: gemtuzumab, lintuzumab, vadastuximab, CIK-CAR.CD33; CD33CART, AMG-330 (CD33/CD3), AMG-673 (CD33/CD3), and GEM-333 (CD3/CD33), and IMGN-779.

In various embodiments, the agent that inhibits binding between CD47 and SIRPα (e.g., magrolimab); and the VEGFA/VEGFR inhibiting agent (e.g., bevacizumab), as described herein, is further combined with an anti-CD37 agent or antibody. Examples of anti-CD37 agents or antibodies that can be co-administered include without limitation: BI836826 (Boehringer Ingelheim), Otlertuzumab, and TRU-016 (Trubion Pharmaceuticals).

In various embodiments, the agent that inhibits binding between CD47 and SIRPα (e.g., magrolimab); and the VEGFA/VEGFR inhibiting agent (e.g., bevacizumab), as described herein, is further combined with an anti-CD38 agent or antibody. Examples of anti-CD38 agents or antibodies that can be co-administered include without limitation: CD38, such as T-007, UCART-38; Darzalex (Genmab), Daratumumab, JNJ-54767414 (Darzalex/Genmab), Isatuximab, SAR650984 (ImmunoGen), MOR202, MOR03087 (MorphoSys), TAK-079; and anti-CD38-attenukine, such as TAK573.

In various embodiments, the agent that inhibits binding between CD47 and SIRPα (e.g., magrolimab); and the VEGFA/VEGFR inhibiting agent (e.g., bevacizumab), as described herein, is further combined with an anti-CD52 agent or antibody. Examples of anti-CD52 agents or antibodies that can be co-administered include without limitation: anti-CD52 antibodies, such as Alemtuzumab (Campath/University of Cambridge).

In various embodiments, the agent that inhibits binding between CD47 and SIRPα (e.g., magrolimab); and the VEGFA/VEGFR inhibiting agent (e.g., bevacizumab), as described herein, is further combined with an anti-CD98 (4F2, FRP-1) agent or antibody. Examples of anti-CD98 agents or antibodies that can be co-administered include without limitation: IGN523 (Igenica).

In various embodiments, the agent that inhibits binding between CD47 and SIRPα (e.g., magrolimab); and the VEGFA/VEGFR inhibiting agent (e.g., bevacizumab), as described herein, is further combined with an anti-CD157 (BST-1) agent or antibody. Examples of anti-CD157 agents or antibodies that can be co-administered include without limitation: OBT357, MEN1112 (Menarini; Oxford BioTherapeutics).

In various embodiments, the agent that inhibits binding between CD47 and SIRPα (e.g., magrolimab); and the VEGFA/VEGFR inhibiting agent (e.g., bevacizumab), as described herein, is further combined with an anti-DKK-1 agent or antibody. Examples of anti-DKK-1 agents or antibodies that can be co-administered include without limitation: BHQ880 (MorphoSys; Novartis), and DKN-01, LY-2812176 (Eli Lilly).

In various embodiments, the agent that inhibits binding between CD47 and SIRPα (e.g., magrolimab); and the VEGFA/VEGFR inhibiting agent (e.g., bevacizumab), as described herein, is further combined with an anti-GRP78 (BiP) agent or antibody. Examples of anti-GRP78 agents or antibodies that can be co-administered include without limitation: PAT-SM6 (OncoMab GmbH).

In various embodiments, the agent that inhibits binding between CD47 and SIRPα (e.g., magrolimab); and the VEGFA/VEGFR inhibiting agent (e.g., bevacizumab), as described herein, is further combined with an anti-NOTCH1 agent or antibody. Examples of anti-NOTCH1 agents or antibodies that can be co-administered include without limitation: Brontictuzumab, OMP-52M51 (OncoMed Pharmaceuticals).

In various embodiments, the agent that inhibits binding between CD47 and SIRPα (e.g., magrolimab); and the VEGFA/VEGFR inhibiting agent (e.g., bevacizumab), as described herein, is further combined with an anti-ROR1 agent or antibody. Examples of anti-ROR1 agents or antibodies that can be co-administered include without limitation: Mapatumumab, TRM1, and HGS-1012 (Cambridge Antibody Technology).

In various embodiments, the agent that inhibits binding between CD47 and SIRPα (e.g., magrolimab); and the VEGFA/VEGFR inhibiting agent (e.g., bevacizumab), as described herein, is further combined with an anti-SLAMF7 (CS1, CD319) agent or antibody. Examples of anti-SLAMF7 agents or antibodies that can be co-administered include without limitation: Elotuzumab, HuLuc63, BMS-901608 (Empliciti/PDL BioPharma), Mogamulizumab (KW-0761).

In various embodiments, the agent that inhibits binding between CD47 and SIRPα (e.g., magrolimab); and the VEGFA/VEGFR inhibiting agent (e.g., bevacizumab), as described herein, is further combined with an anti-TNFRSF10A (DR4; APO2; CD261; TRAILR1; TRAILR-1) agent or antibody. Examples of anti-TNFRSF10A agents or antibodies that can be co-administered include without limitation: Mapatumumab, TRM1, and HGS-1012 (Cambridge Antibody Technology).

In various embodiments, the agent that inhibits binding between CD47 and SIRPα (e.g., magrolimab); and the VEGFA/VEGFR inhibiting agent (e.g., bevacizumab), as described herein, is further combined with an anti-Transferrin Receptor (TFRC; CD71) agent or antibody. Examples of anti-Transferrin Receptor agents or antibodies that can be co-administered include without limitation: E2.3/A27.15 (University of Arizona).

In various embodiments, the agent that inhibits binding between CD47 and SIRPα (e.g., magrolimab); and the VEGFA/VEGFR inhibiting agent (e.g., bevacizumab), as described herein, is further combined with an anti-EPHA3 agent or antibody. Examples of anti-EPHA3 agents or antibodies that can be co-administered include without limitation: Ifabotuzumab, KB004 (Ludwig Institute for Cancer Research).

In various embodiments, the agent that inhibits binding between CD47 and SIRPα (e.g., magrolimab); and the VEGFA/VEGFR inhibiting agent (e.g., bevacizumab), as described herein, is further combined with an anti-CCR4 agent or antibody. Examples of anti-CCR4 agents or antibodies that can be co-administered include without limitation: Mogamulizumab, KW-0761 (Poteligeo/Kyowa Hakko Kirin Co.).

In various embodiments, the agent that inhibits binding between CD47 and SIRPα (e.g., magrolimab); and the VEGFA/VEGFR inhibiting agent (e.g., bevacizumab), as described herein, is further combined with an anti-CXCR4 agent or antibody. Examples of anti-CXCR4 agents or antibodies that can be co-administered include without limitation: Ulocuplumab, BMS-936564, MDX-1338 (Medarex), and PF-06747143 (Pfizer).

In various embodiments, the agent that inhibits binding between CD47 and SIRPα (e.g., magrolimab); and the VEGFA/VEGFR inhibiting agent (e.g., bevacizumab), as described herein, is further combined with an anti-BAFF agent or antibody. Examples of anti-BAFF agents or antibodies that can be co-administered include without limitation: Tabalumab, LY2127399 (Eli Lilly).

In various embodiments, the agent that inhibits binding between CD47 and SIRPα (e.g., magrolimab); and the VEGFA/VEGFR inhibiting agent (e.g., bevacizumab), as described herein, is further combined with an anti-BAFF Receptor (BAFF-R) agent or antibody. Examples of anti-BAFF-R agents or antibodies that can be co-administered include without limitation: VAY736 (MorphoSys; Novartis).

In various embodiments, the agent that inhibits binding between CD47 and SIRPα (e.g., magrolimab); and the VEGFA/VEGFR inhibiting agent (e.g., bevacizumab), as described herein, is further combined with an anti-RANKL agent or antibody. Examples of anti-RANKL agents or antibodies that can be co-administered include without limitation: Denosumab, AMG-162 (Prolia; Ranmark; Xgeva/Amgen).

In various embodiments, the agent that inhibits binding between CD47 and SIRPα (e.g., magrolimab); and the VEGFA/VEGFR inhibiting agent (e.g., bevacizumab), as described herein, is further combined with an anti-IL-6 agent or antibody. Examples of anti-IL-6 agents or antibodies that can be co-administered include without limitation: Siltuximab, CNTO-328 (Sylvant/Centocor).

In various embodiments, the agent that inhibits binding between CD47 and SIRPα (e.g., magrolimab); and the VEGFA/VEGFR inhibiting agent (e.g., bevacizumab), as described herein, is further combined with an anti-IL-6 Receptor (IL-6R) agent or antibody. Examples of anti-IL-6R agents or antibodies that can be co-administered include without limitation: Tocilizumab, R-1569 (Actemra/Chugai Pharmaceutical; Osaka University), or AS-101 (CB-06-02, IVX-Q-101).

In various embodiments, the agent that inhibits binding between CD47 and SIRPα (e.g., magrolimab); and the VEGFA/VEGFR inhibiting agent (e.g., bevacizumab), as described herein, is further combined with an anti-IL3RA (CD123) agent or antibody. Examples of anti-IL3RA (CD123) agents or antibodies that can be co-administered include without limitation: tagraxofusp, talacotuzumab (JNJ-56022473; CSL362 (CSL)), pivekimab sunirine (IMGN632), MB-102 (Mustang Bio), CSL360 (CSL); vibecotamab (XmAb14045; Xencor); KHK2823 (Kyowa Hakko Kirin Co.); MGD-024 (CD123/CD3; Macrogenics), APV0436 (CD123/CD3); flotetuzumab (CD123/CD3); JNJ-63709178 (CD123/CD3); and XmAb-14045 (CD123/CD3) (Xencor).

In various embodiments, the agent that inhibits binding between CD47 and SIRPα (e.g., magrolimab); and the VEGFA/VEGFR inhibiting agent (e.g., bevacizumab), as described herein, is further combined with an anti-IL2RA (CD25) agent or antibody. Examples of anti-IL2RA agents or antibodies that can be co-administered include without limitation: Basiliximab, SDZ-CHI-621 (Simulect/Novartis), and Daclizumab.

In various embodiments, the agent that inhibits binding between CD47 and SIRPα (e.g., magrolimab); and the VEGFA/VEGFR inhibiting agent (e.g., bevacizumab), as described herein, is further combined with an anti-IGF-1R (CD221) agent or antibody. Examples of anti-IGF-1R agents or antibodies that can be co-administered include without limitation: Ganitumab, AMG-479 (Amgen); Ganitumab, AMG-479 (Amgen), Dalotuzumab, MK-0646 (Pierre Fabre), and AVE1642 (ImmunoGen).

In various embodiments, the agent that inhibits binding between CD47 and SIRPα (e.g., magrolimab); and the VEGFA/VEGFR inhibiting agent (e.g., bevacizumab), as described herein, is further combined with an anti-GM-CSF (CSF2) agent or antibody. Examples of anti-GM-CSF agents or antibodies that can be co-administered include without limitation: Lenzilumab (a.k.a., KB003; KaloBios Pharmaceuticals).

In various embodiments, the agent that inhibits binding between CD47 and SIRPα (e.g., magrolimab); and the VEGFA/VEGFR inhibiting agent (e.g., bevacizumab), as described herein, is further combined with an anti-HGF agent or antibody. Examples of anti-HGF agents or antibodies that can be co-administered include without limitation: Ficlatuzumab, AV-299 (AVEO Pharmaceuticals).

In various embodiments, the agent that inhibits binding between CD47 and SIRPα (e.g., magrolimab); and the VEGFA/VEGFR inhibiting agent (e.g., bevacizumab), as described herein, is further combined with an anti-CD44 agent or antibody. Examples of anti-CD44 agents or antibodies that can be co-administered include without limitation: RG7356, R05429083 (Chugai Biopharmaceuticals; Roche).

In various embodiments, the agent that inhibits binding between CD47 and SIRPα (e.g., magrolimab); and the VEGFA/VEGFR inhibiting agent (e.g., bevacizumab), as described herein, is further combined with an anti-VLA-4 (CD49d) agent or antibody. Examples of anti-VLA-4 agents or antibodies that can be co-administered include without limitation: Natalizumab, BG-0002-E (Tysabri/Elan Corporation).

In various embodiments, the agent that inhibits binding between CD47 and SIRPα (e.g., magrolimab); and the VEGFA/VEGFR inhibiting agent (e.g., bevacizumab), as described herein, is further combined with an anti-ICAM-1 (CD54) agent or antibody. Examples of anti-ICAM-1 agents or antibodies that can be co-administered include without limitation: BI-505 (BioInvent International).

In various embodiments, the agent that inhibits binding between CD47 and SIRPα (e.g., magrolimab); and the VEGFA/VEGFR inhibiting agent (e.g., bevacizumab), as described herein, is further combined with an anti-VEGF-A agent or antibody. Examples of anti-VEGF-A agents or antibodies that can be co-administered include without limitation: Bevacizumab (Avastin/Genentech; Hackensack University Medical Center).

In various embodiments, the agent that inhibits binding between CD47 and SIRPα (e.g., magrolimab); and the VEGFA/VEGFR inhibiting agent (e.g., bevacizumab), as described herein, is further combined with an anti-Endosialin (CD248, TEM1) agent or antibody. Examples of anti-Endosialin agents or antibodies that can be co-administered include without limitation: Ontecizumab, MORAB-004 (Ludwig Institute for Cancer Research; Morphotek).

In various embodiments, the agent that inhibits binding between CD47 and SIRPα (e.g., magrolimab); and the VEGFA/VEGFR inhibiting agent (e.g., bevacizumab), as described herein, is further combined with an anti-CD79 agent or antibody. Examples of anti-CD79 agents or antibodies that can be co-administered include without limitation: polatuzumab, DCDS4501A, RG7596 (Genentech).

In various embodiments, the agent that inhibits binding between CD47 and SIRPα (e.g., magrolimab); and the VEGFA/VEGFR inhibiting agent (e.g., bevacizumab), as described herein, is further combined with an anti-Isocitrate dehydrogenase (IDH) agent or antibody. Examples of anti-IDH agents or antibodies that can be co-administered include without limitation: IDH1 inhibitor ivosidenib (Tibsovo; Agios) and the IDH2 inhibitor enasidenib (Idhifa; Celgene/Agios).

In various embodiments, the agent that inhibits binding between CD47 and SIRPα (e.g., magrolimab); and the VEGFA/VEGFR inhibiting agent (e.g., bevacizumab), as described herein, is further combined with an antibody that targets tumor associated calcium signal transducer 2 (TACSTD2) (NCBI Gene ID: 4070; EGP-1, EGP1, GA733-1, GA7331, GP50, M1S1, TROP2), such as sacituzumab, e.g., sacituzumab govitecan (TRODELVY™).

In various embodiments, the agent that inhibits binding between CD47 and SIRPα (e.g., magrolimab); and the VEGFA/VEGFR inhibiting agent (e.g., bevacizumab), as described herein, is further combined with an anti-major histocompatibility complex, class I, G (HLA-G; NCBI Gene ID: 3135) antibody, such as TTX-080.

In various embodiments, the agent that inhibits binding between CD47 and SIRPα (e.g., magrolimab); and the VEGFA/VEGFR inhibiting agent (e.g., bevacizumab), as described herein, is further combined with an anti-leukocyte immunoglobulin like receptor B2 (LILRB2, a.k.a., CD85D, ILT4; NCBI Gene ID: 10288) antibody, such as JTX-8064 or MK-4830.

### TNF Receptor Superfamily (TNFRSF) Member Agonists or Activators

In various embodiments, the agent that inhibits binding between CD47 and SIRPα (e.g., magrolimab); and the VEGFA/VEGFR inhibiting agent (e.g., bevacizumab), as described herein, is further combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628, each of which is hereby incorporated by reference in its entirety.

Examples anti-TNF receptor superfamily member 10b (TNFRSF10B, DR5, TRAILR2) antibodies that can be co-administered include without limitation, such as DS-8273, CTB-006, INBRX-109, and GEN-1029.

Examples of anti-TNFRSF5 (CD40) antibodies that can be co-administered include without limitation selicrelumab (R07009789), mitazalimab (a.k.a., vanalimab, ADC-1013, JNJ-64457107), RG7876, SEA-CD40, APX-005M and ABBV-428, ABBV-927, and JNJ-64457107.

Examples of anti-TNFRSF7 (CD27) that can be co-administered include without limitation varlilumab (CDX-1127).

Examples of anti-TNFRSF9 (4-1BB, CD137) antibodies that can be co-administered include without limitation urelumab, utomilumab (PF-05082566), AGEN2373, and ADG-106, BT-7480, and QL1806.

Examples of anti-TNFRSF17 (BCMA) that can be co-administered include without limitation GSK-2857916.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628, each of which is hereby incorporated by reference in its entirety.

Example anti-TRAILR1, anti-TRAILR2, anti-TRAILR3, anti-TRAILR4 antibodies that can be co-administered include without limitation ABBV-621.

Examples of Bi-specific antibodies targeting TNFRSF family members that can be co-administered include without limitation PRS-343 (CD-137/HER2), AFM26 (BCMA/CD16A), AFM-13 (CD16/CD30), REGN-1979 (CD20/CD3), AMG-420 (BCMA/CD3), INHIBRX-105 (4-1BB/PDL1), FAP-4-IBBL (4-1BB/FAP), XmAb-13676 (CD3/CD20), RG-7828 (CD20/CD3), CC-93269 (CD3/BCMA), REGN-5458 (CD3/BCMA), and IMM-0306 (CD47/CD20), and AMG-424 (CD38.CD3).

Examples of inhibitors of PVR related immunoglobulin domain containing (PVRIG, CD112R) that can be co-administered include without limitation: COM-701.

Examples of inhibitors of T cell immunoreceptor with Ig and ITIM domains (TIGIT; NCBI Gene ID: 201633) that can be co-administered include without limitation: BMS-986207, RG-6058, AGEN-1307, and COM-902, etigilimab, tiragolumab (a.k.a., MTIG-7192A; RG-6058; RO 7092284), AGEN1777, IBI-939, AB154, MG1131 and EOS884448 (EOS-448).

Examples of inhibitors of hepatitis A virus cellular receptor 2 (HAVCR2, TIMD3, TIM-3) that can be co-administered include without limitation: cobolimab (TSR-022), LY-3321367, sabatolimab (MBG-453), INCAGN-2390, RO-7121661 (PD-1/TIM-3), LY-3415244 (TIM-3/PDL1), and RG7769 (PD-1/TIM-3).

Examples of inhibitors of lymphocyte activating 3 (LAG-3, CD223) that can be co-administered include without limitation: relatlimab (ONO-4482), LAG-525, MK-4280, REGN-3767, INCAGN2385, TSR-033, MGD-013 (PD-1/LAG-3), and FS-118 (LAG-3/PD-L1).

Examples of anti-killer cell immunoglobulin like receptor, three Ig domains and long cytoplasmic tail 1 (KIR3DL1; KIR; NCBI Gene ID: 3811) monoclonal antibodies, such as lirilumab (IPH-2102), and IPH-4102.

Examples of anti-NKG2a antibodies that can be co-administered include without limitation: monalizumab.

Examples of anti-V-set immunoregulatory receptor (VSIR, B7H5, VISTA) antibodies that can be co-administered include without limitation: HMBD-002, and CA-170 (PD-L1/VISTA).

Examples of anti-CD70 antibodies that can be co-administered include without limitation: AMG-172.

Examples of anti-ICOS antibodies that can be co-administered include without limitation: JTX-2011, GSK3359609.

Examples of ICOS agonists that can be co-administered include without limitation: ICOS-L.COMP (Gariepy, et al. 106th Annu Meet Am Assoc Immunologists (AAI) (May 9-13, San Diego) 2019, Abst 71.5).

### Immune Checkpoint Inhibitors

In various embodiments, the agent that inhibits binding between CD47 and SIRPα (e.g., magrolimab); and the VEGFA/VEGFR inhibiting agent (e.g., bevacizumab), as described herein, is further combined with one or more immune checkpoint inhibitors. In some embodiments, the one or more immune checkpoint inhibitors is a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, HBM-4003, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors/antibodies of PD-L1 (CD274) or PD-1 (PDCD1) that can be co-administered include without limitation zimberelimab, pembrolizumab (KEYTRUDA®, MK-3477), nivolumab (OPDIVO®, BMS-936558, MDX-1106), cemiplimab, pidilizumab, spartalizumab (PDR-001), atezolizumab (RG-7446; TECENTRIQ, MPDL3280A), durvalumab (MEDI-4736), avelumab (MSB0010718C), tislelizumab (BGB-A317), toripalimab (JS-001), genolimzumab (CBT-501), camrelizumab (SHR-1210), dostarlimab (TSR-042), sintilimab (IBI-308), tislelizumab (BGB-A317), cemiplimab (REGN-2810), lambrolizumab (CAS Reg. No. 1374853-91-4), AMG-404, AMP-224, MEDI0680 (AMP-514), BMS-936559, CK-301, PF-06801591, GEN-1046 (PD-L1/4-1BB), GLS-010 (WBP-3055), AK-103 (HX-008), AK-105, CS-1003, HLX-10, MGA-012, BI-754091, AGEN-2034, JNJ-63723283, LZM-009, BCD-100, LY-3300054, SHR-1201, Sym-021, ABBV-181, PD1-PIK, BAT-1306, CX-072, CBT-502, MSB-2311, JTX-4014, BGB-A333, SHR-1316, CS-1001 (WBP-3155, KN-035, HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), RO-7247669 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM-3/PDL1), RG7769 (PD-1/TIM-3) and INBRX-105 (4-1BB/PDL1), GNS-1480 (PD-L1/EGFR), SCH-900475, PF-06801591, AGEN-2034, AK-105, PD1-PIK, BAT-1306, BMS-936559, CK-301, MEDI-0680, PDR001+Tafinlar®+Mekinist®, and those described, e.g., in Intl. Patent Publ. Nos. WO2018195321, WO2020014643, WO2019160882, and WO2018195321.

In various embodiments, an anti-CD47 agent as described herein, is combined with an inhibitor of MCL1 apoptosis regulator, BCL2 family member (MCL1, TM; EAT; MCL1L; MCL1S; Mel-1; BCL2L3; MCL1-ES; bcl2-L-3; mcl1/EAT; NCBI Gene ID: 4170). Examples of MCL1 inhibitors include AMG-176, AMG-397, 5-64315, and AZD-5991, 483-LM, A-1210477, UMI-77, JKY-5-037, and those described in WO2018183418, WO2016033486, and WO2017147410.

### Toll-Like Receptor (TLR) Agonists

In various embodiments, an anti-CD47 agent or an anti-SIRPα agent as described herein, is combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation DS-0509, GS-9620, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10 and PUL-042. Examples of TLR3 agonist include rintatolimod, poly-ICLC, RIBOXXON®, Apoxxim, RIBOXXIM®, IPH-33, MCT-465, MCT-475, and ND-1.1.

Examples of TLR8 inhibitors include, but are not limited to, E-6887, IMO-8400, IMO-9200 and VTX-763.

Examples of TLR8 agonists include, but are not limited to, MCT-465, motolimod, GS-9688, and VTX-1463.

Examples of TLR9 agonists include but are not limited to, AST-008, IMO-2055, IMO-2125, lefitolimod, litenimod, MGN-1601, and PUL-042.

Examples of TLR7/TLR8 agonists include without limitation NKTR-262, IMO-4200, MEDI-9197 (telratolimod) and resiquimod.

Examples of TLR agonists include without limitation: lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod.

In some embodiments, the therapeutic agent is a stimulator of interferon genes (STING) In some embodiments, the STING receptor agonist or activator is selected from ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP), and cyclic-di-AMP.

### TCR Signaling Modulators

In various embodiments, the agent that inhibits binding between CD47 and SIRPα (e.g., magrolimab); and the VEGFA/VEGFR inhibiting agent (e.g., bevacizumab), as described herein, is further combined with one or more agonist or antagonist of T-Cell Receptor (TCR) signaling modulators. Activation of T cells through the TCR and is essential for thymocyte development and effector T cell function. TCR activation promotes signaling cascades that ultimately determine cell fate through regulating cytokine production, cell survival, proliferation, and differentiation. Examples of TCR signaling modulators include without limitation CD2 (cluster of differentiation 2, LFA-2, T11, LFA-3 receptor), CD3 (cluster of differentiation 3), CD4 (cluster of differentiation 4), CD8 (cluster of differentiation 8), CD28 (cluster of differentiation 28), CD45 (PTPRC, B220, GP180), LAT (Linker for activation of T cells, LAT1), Lck, LFA-1 (ITGB2, CD18, LAD, LCAMB), Src, Zap-70, SLP-76, DGKalpha, CBL-b, CISH, HPK1. Examples of agonist of cluster of differentiation 3 (CD3) that can be co-administered include without limitation MGD015.

In various embodiments, the agent that inhibits binding between CD47 and SIRPα (e.g., magrolimab); and the VEGFA/VEGFR inhibiting agent (e.g., bevacizumab), as described herein, is further combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of cancer cells within the tumor microenvironment. Activation or stimulation of stimulatory immune check points can augment the effect of immune checkpoint inhibitors in cancer therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (PDL1, PD-L1); programmed cell death 1 (PDCD1, PD-1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM-3); galectin 9 (LGALS9); lymphocyte activating 3 (LAG-3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor(KIR); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1).

In various embodiments, the agent that inhibits binding between CD47 and SIRPα (e.g., magrolimab); and the VEGFA/VEGFR inhibiting agent (e.g., bevacizumab), as described herein, is further combined with one or more blockers or inhibitors of one or more T-cell inhibitory immune checkpoint proteins or receptors. Illustrative T-cell inhibitory immune checkpoint proteins or receptors include without limitation CD274 (PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG-3, CD223); hepatitis A virus cellular receptor 2 (HAVCR2, TIMD3, TIM-3); galectin 9 (LGALS9); killer cell immunoglobulin like receptor(KIR); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

In various embodiments, the agent that inhibits binding between CD47 and SIRPα (e.g., magrolimab); and the VEGFA/VEGFR inhibiting agent (e.g., bevacizumab), as described herein, is further combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In various embodiments, the agent that inhibits binding between CD47 and SIRPα (e.g., magrolimab); and the VEGFA/VEGFR inhibiting agent (e.g., bevacizumab), as described herein, is further combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94).

In various embodiments, the agent that inhibits binding between CD47 and SIRPα (e.g., magrolimab); and the VEGFA/VEGFR inhibiting agent (e.g., bevacizumab), as described herein, is further combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

### Adenosine Generation and Signaling

In various embodiments, the agent that inhibits binding between CD47 and SIRPα (e.g., magrolimab); and the VEGFA/VEGFR inhibiting agent (e.g., bevacizumab), as described herein, is further combined with an agonist or antagonist of A1R, A2AR, A2BR, A3R, CD73, CD39, CD26; e.g., Adenosine A3 receptor (A3R) agonists, such as namodenoson (CF102); A2aR/A2bR antagonists, such as AB928; anti-CD73 antibodies, such as MEDI-9447 (oleclumab), CPX-006, IPH-53, BMS-986179, NZV-930, CPI-006; CD73 inhibitors, such as AB-680, PSB-12379, PSB-12441, PSB-12425, CB-708, and those described in Int Patent Publication No. WO19173692; CD39/CD73 inhibitors, such as PBF-1662; anti-CD39 antibodies, such as TTX-030; adenosine A2A receptor antagonists, such as CPI-444, AZD-4635, preladenant, PBF-509; and adenosine deaminase inhibitors, such as pentostatin, cladribine.

### Bi-Specific T-Cell Engagers

In various embodiments, the agent that inhibits binding between CD47 and SIRPα (e.g., magrolimab); and the VEGFA/VEGFR inhibiting agent (e.g., bevacizumab), as described herein, is further combined with a bi-specific T-cell engager (e.g., not having an Fc) or an anti-CD3 bi-specific antibody (e.g., having an Fc). Illustrative anti-CD3 bi-specific antibodies or BiTEs that can be co-administered include AMG-160 (PSMA/CD3), AMG-212 (PSMA/CD3), AMG-330 (CD33/CD3), AMG-420 (BCMA/CD3), AMG-427 (FLT3/CD3), AMG-562 (CD19/CD3), AMG-596 (EGFRvIII/CD3), AMG-701 (BCMA/CD3), AMG-757 (DLL3/CD3), JNJ-64052781 (CD19/CD3), AMG-211 (CEA/CD3), BLINCYTO® (CD19/CD3), RG7802 (CEA/CD3), ERY-974 (CD3/GPC3), huGD2-BsAb (CD3/GD2), PF-06671008 (Cadherins/CD3), APV0436 (CD123/CD3), ERY974, flotetuzumab (CD123/CD3), GEM333 (CD3/CD33), GEMoab (CD3/PSCA), REGN-1979 (CD20/CD3), REGN-5678 (PSMA/CD28), MCLA-117 (CD3/CLEC12A), JNJ-0819, JNJ-7564 (CD3/heme), JNJ-63709178 (CD123/CD3), MGD-007 (CD3/gpA33), MGD-009 (CD3/B7H3), IMCgp100 (CD3/gp100), XmAb-14045 (CD123/CD3), XmAb-13676 (CD3/CD20), XmAb-18087 (SSTR2/CD3), catumaxomab (CD3/EpCAM), REGN-4018 (MUC16/CD3), RG6026, RG6076, RG6194, RG-7828 (CD20/CD3), CC-93269 (CD3/BCMA), REGN-5458 (CD3/BCMA), GRB-1302 (CD3/Erbb2), GRB-1342 (CD38/CD3), PF-06863135 (BCMA/CD3), SAR440234 (CD3/CDw123). As appropriate, the anti-CD3 binding bi-specific molecules may or may not have an Fc. Illustrative bi-specific T-cell engagers that can be co-administered target CD3 and a tumor-associated antigen as described herein, including, e.g., CD19 (e.g., blinatumomab); CD33 (e.g., AMG330); CEA (e.g., MEDI-565); receptor tyrosine kinase-like orphan receptor 1 (ROR1) (Gohil, et al., Oncoimmunology. (2017) May 17; 6(7):e1326437); PD-L1 (Horn, et al., Oncotarget. 2017 Aug. 3; 8(35):57964-57980); and EGFRvIII (Yang, et al., Cancer Lett. 2017 Sep. 10; 403:224-230).

### Bi- and Tri-Specific Natural Killer (NK)-Cell Engagers

In various embodiments, the agent that inhibits binding between CD47 and SIRPα (e.g., magrolimab); and the VEGFA/VEGFR inhibiting agent (e.g., bevacizumab), as described herein, is further combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. Illustrative bi-specific NK-cell engagers that can be co-administered target CD16 and one or more tumor-associated antigens as described herein, including, e.g., CD19, CD20, CD22, CD30, CD33, CD123, EGFR, EpCAM, ganglioside GD2, HER2/neu, HLA Class II and FOLR1. BiKEs and TriKEs are described, e.g., in Felices, et al., Methods Mol Biol. (2016) 1441:333-346; Fang, et al., Semin Immunol. (2017) 31:37-54.

### Hematopoietic Progenitor Kinase 1 (HPK1) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO-2018183956, WO-2018183964, WO-2018167147, WO-2018183964, WO-2016205942, WO-2018049214, WO-2018049200, WO-2018049191, WO-2018102366, WO-2018049152, WO2020092528, WO2020092621 and WO-2016090300.

### Apoptosis Signal-Regulating Kinase (ASK) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of an ASK inhibitor, e.g., mitogen-activated protein kinase kinase kinase 5 (MAP3K5; ASK1, MAPKKK5, MEKK5; NCBI Gene ID: 4217). Examples of ASK1 inhibitors include without limitation, those described in WO 2011/008709 (Gilead Sciences) and WO 2013/112741 (Gilead Sciences).

### Bruton Tyrosine Kinase (BTK) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include without limitation, (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, Calquence+AZD6738, Calquence+danvatirsen.

### Cyclin-dependent Kinase (CDK) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of cyclin dependent kinase 1 (CDK1, CDC2; CDC28A; P34CDC2; NCBI Gene ID: 983); cyclin dependent kinase 2 (CDK2, CDKN2; p33(CDK2); NCBI Gene ID: 1017); cyclin dependent kinase 3 (CDK3; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022); cyclin dependent kinase 9 (CDK9, TAK; C-2k; CTK1; CDC2L4; PITALRE; NCBI Gene ID: 1025). Inhibitors of CDK 1, 2, 3, 4, 6, 7 and/or 9, include without limitation abemaciclib, alvocidib (HMR-1275, flavopiridol), AT-7519, dinaciclib, ibrance, FLX-925, LEE001, palbociclib, ribociclib, rigosertib, selinexor, UCN-01, SY1365, CT-7001, SY-1365, G1T38, milciclib, trilaciclib, PF-06873600, AZD4573, and TG-02.

### Discoidin Domain Receptor (DDR) Inhibitors.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include without limitation, dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

### Histone Deacetylase (HDAC) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, entinostat, romidepsin, tucidinostat.

### Indoleamine-Pyrrole-2,3-Dioxygenase (IDO1) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

### Janus Kinase (JAK) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718). Examples of JAK inhibitors include without limitation, AT9283, AZD1480, baricitinib, BMS-911543, fedratinib, filgotinib (GLPG0634), gandotinib (LY2784544), INCB039110 (itacitinib), lestaurtinib, momelotinib (CYT0387), NS-018, pacritinib (SB1518), peficitinib (ASP015K), ruxolitinib, tofacitinib (formerly tasocitinib), INCB052793, and XL019.

### Matrix Metalloprotease (MMP) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148). Examples of MMP9 inhibitors include without limitation, marimastat (BB-2516), cipemastat (Ro 32-3555), GS-5745 (andecaliximab) and those described in WO 2012/027721 (Gilead Biologics).

### RAS and RAS Pathway Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of KRAS proto-oncogene, GTPase (KRAS; a.k.a., NS; NS3; CFC2; RALD; K-Ras; KRAS1; KRAS2; RASK2; KI-RAS; C—K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893); HRas proto-oncogene, GTPase (HRAS; a.k.a., CTLO; KRAS; HAMSV; HRAS1; KRAS2; RASH1; RASK2; Ki-Ras; p21ras; C—H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of KRAS. Examples of KRAS inhibitors include AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 (Ac-RRCPLYISYDPVCRR-NH2) (SEQ ID NO: 256) and KRpep-2d (Ac-RRRRCPLYISYDPVCRRRR-NH2) (SEQ ID NO: 257).

In various embodiments, the agent that inhibits binding between CD47 and SIRPα (e.g., magrolimab); and the VEGFA/VEGFR inhibiting agent (e.g., bevacizumab), as described herein, is further combined with an inhibitor of KRAS mRNA. Illustrative KRAS mRNA inhibitors include anti-KRAS U1 adaptor, AZD-4785, siG12D-LODER™, and siG12D exosomes.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of MEK. Illustrative MEK inhibitors that can be co-administered include binimetinib, cobimetinib, PD-0325901, pimasertib, RG-7304, selumetinib, trametinib, and selumetinib.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of AKT. Illustrative AKT inhibitors that can be co-administered include RG7440, MK-2206, ipatasertib, afuresertib, AZD5363, and ARQ-092, capivasertib, triciribine, ABTL-0812 (PI3K/Akt/mTOR).

In various embodiments, the agent that inhibits binding between CD47 and SIRPα (e.g., magrolimab); and the VEGFA/VEGFR inhibiting agent (e.g., bevacizumab), as described herein, is further combined with an inhibitor of Raf. Illustrative Raf inhibitors that can be co-administered BGB-283 (Raf/EGFR), HM-95573, LXH-254, LY-3009120, RG7304, TAK-580, dabrafenib, vemurafenib, encorafenib (LGX818), PLX8394. RAF-265 (Raf/VEGFR), ASN-003 (Raf/PI3K).

In various embodiments, the agent that inhibits binding between CD47 and SIRPα (e.g., magrolimab); and the VEGFA/VEGFR inhibiting agent (e.g., bevacizumab), as described herein, is further combined with an inhibitor of ERK. Illustrative ERK inhibitors that can be co-administered include LTT-462, LY-3214996, MK-8353, ravoxertinib, GDC-0994, and ulixertinib.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of PI3K. Illustrative PI3K inhibitors that can be co-administered include idelalisib (Zydelig®), alpelisib, buparlisib, pictilisib, eganelisib (IPI-549). Illustrative PI3K/mTOR inhibitors that can be co-administered include dactolisib, omipalisib, voxtalisib, gedatolisib, GSK2141795, RG6114.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of mTOR. Illustrative mTOR inhibitors that can be co-administered include as sapanisertib, vistusertib (AZD2014), ME-344, sirolimus (oral nano-amorphous formulation, cancer), TYME-88 (mTOR/cytochrome P450 3A4).

In certain embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov. 1; 408:130-137. Also, K-RAS and mutant N-RAS can be reduced by the irreversible ERBB1/2/4 inhibitor neratinib. See, e.g., Booth, et al., Cancer Biol Ther. 2018 Feb. 1; 19(2):132-137.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of RAS. Examples of RAS inhibitors include NEO-100 and rigosertib.

In various embodiments, the agent that inhibits binding between CD47 and SIRPα (e.g., magrolimab); and the VEGFA/VEGFR inhibiting agent (e.g., bevacizumab), as described herein, is further combined with an antagonist of EGFR, such as AMG-595, necitumumab, ABBV-221, depatuxizumab mafodotin (ABT-414), tomuzotuximab, ABT-806, vectibix, modotuximab, RM-1929.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, SAR442720 and those described in WO2018172984 and WO2017211303.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of mitogen-activated protein kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, CK-127, cobimetinib (GDC-0973, XL-518), MT-144, selumetinib (AZD6244), sorafenib, trametinib (GSK1120212), uprosertib+trametinib, PD-0325901, pimasertib, LTT462, AS703988, CC-90003, refametinib, TAK-733, CI-1040, RG7421.

In various embodiments, the agent that inhibits binding between CD47 and SIRPα (e.g., magrolimab); and the VEGFA/VEGFR inhibiting agent (e.g., bevacizumab), as described herein, is further combined with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PI3K inhibitor is a pan-PI3K inhibitor. Examples of PI3K inhibitors include without limitation, ACP-319, AEZA-129, AMG-319, AS252424, AZD8186, BAY 1082439, BEZ235, bimiralisib (PQR309), buparlisib (BKM120), BYL719 (alpelisib), carboxyamidotriazole orotate (CTO), CH5132799, CLR-457, CLR-1401, copanlisib (BAY 80-6946), DS-7423, dactolisib, duvelisib (IPI-145), fimepinostat (CUDC-907), gedatolisib (PF-05212384), GDC-0032, GDC-0084 (RG7666), GDC-0077, pictilisib (GDC-0941), GDC-0980, GSK2636771, GSK2269577, GSK2141795, idelalisib (Zydelig®), INCB040093, INCB50465, IPI-443, IPI-549, KAR4141, LY294002, LY3023414, NERLYNX® (neratinib), nemiralisib (GSK2269557), omipalisib (GSK2126458, GSK458), OXY111A, panulisib (P7170, AK151761), PA799, perifosine (KRX-0401), Pilaralisib (SAR245408; XL147), puquitinib mesylate (XC-302), SAR260301, seletalisib (UCB-5857), serabelisib (INK-1117,MLN-1117,TAK-117), SF1126, sonolisib (PX-866), RG6114, RG7604, rigosertib sodium (ON-01910 sodium), RP5090, tenalisib (RP6530), RV-1729, SRX3177, taselisib, TG100115, umbralisib (TGR-1202), TGX221, voxtalisib (SAR245409), VS-5584, WX-037, X-339, X-414, XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

### Spleen Tyrosine Kinase (SYK) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of spleen associated tyrosine kinase (SYK, p72-Syk, Gene ID: 6850). Examples of SYK inhibitors include without limitation, 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), and those described in U.S. Pat. No. 8,450,321 (Gilead Connecticut) and those described in U.S. 2015/0175616.

### Tyrosine-Kinase Inhibitors (TKIs)

In various embodiments, the agent that inhibits binding between CD47 and SIRPα (e.g., magrolimab); and the VEGFA/VEGFR inhibiting agent (e.g., bevacizumab), as described herein, is further combined with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation, axitinib, afatinib, ARQ-087 (derazantinib), asp5878, AZD3759, AZD4547, bosutinib, brigatinib, cabozantinib, cediranib, crenolanib, crizotinib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, olmutinib, osimertinib (AZD-9291), pazopanib, ponatinib, poziotinib, quizartinib, radotinib, rociletinib, sulfatinib (HMPL-012), sunitinib, famitinib L-malate, (MAC-4), tivoanib, TH-4000, and MEDI-575 (anti-PDGFR antibody) and TAK-659.

### Chemotherapeutic Agents (Standard of Care)

In various embodiments, the agent that inhibits binding between CD47 and SIRPα (e.g., magrolimab); and the VEGFA/VEGFR inhibiting agent (e.g., bevacizumab), as described herein, is further combined with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer. Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223, 177-Lu-PSMA-617; trichothecenes, especially T-2 toxin, verracurin A, roridin A, and anguidine; taxoids such as paclitaxel (TAXOL®), albumin-bound or nab-paclitaxel (ABRAXANE®), docetaxel (TAXOTERE®), cabazitaxel, BIND-014, tesetaxel; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors. Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®). Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®). Examples of anti-androgens include apalutamide, abiraterone, enzalutamide, flutamide, galeterone, nilutamide, bicalutamide, leuprolide, goserelin, ODM-201, APC-100, ODM-204. An example progesterone receptor antagonist includes onapristone.

### Anti-Angiogenic Agents

In various embodiments, the agent that inhibits binding between CD47 and SIRPα (e.g., magrolimab); and the VEGFA/VEGFR inhibiting agent (e.g., bevacizumab), as described herein, is further combined with an anti-angiogenic agent. Anti-angiogenic agents that can be co-administered include, but are not limited to, retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

### Anti-Fibrotic Agents

In various embodiments, the agent that inhibits binding between CD47 and SIRPα (e.g., magrolimab); and the VEGFA/VEGFR inhibiting agent (e.g., bevacizumab), as described herein, is further combined with an anti-fibrotic agent. Anti-fibrotic agents that can be co-administered include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Anti-Inflammatory Agents

In various embodiments, the agent that inhibits binding between CD47 and SIRPα (e.g., magrolimab); and the VEGFA/VEGFR inhibiting agent (e.g., bevacizumab), as described herein, is further combined with an anti-inflammatory agent. Example anti-inflammatory agents include without limitation inhibitors of one or more of arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240), soluble epoxide hydrolase 2 (EPHX2, SEH; NCBI Gene ID: 2053) and/or mitogen-activated protein kinase kinase kinase 8 (MAP3K8, TPL2; NCBI Gene ID: 1326). In some embodiments, the inhibitor is a dual inhibitor, e.g., a dual inhibitor of COX-2/COX-1, COX-2/SEH, COX-2/CA, COX-2/5-LOX.

Examples of inhibitors of prostaglandin-endoperoxide synthase 1 (PTGS1, COX-1; NCBI Gene ID: 5742) that can be co-administered include without limitation mofezolac, GLY-230, and TRK-700.

Examples of inhibitors of prostaglandin-endoperoxide synthase 2 (PTGS2, COX-2; NCBI Gene ID: 5743) that can be co-administered include without limitation diclofenac, meloxicam, parecoxib, etoricoxib, AP-101, celecoxib, AXS-06, diclofenac potassium, DRGT-46, AAT-076, meisuoshuli, lumiracoxib, meloxicam, valdecoxib, zaltoprofen, nimesulide, Anitrazafen, Apricoxib, Cimicoxib, Deracoxib, Flumizole, Firocoxib, Mavacoxib, NS-398, Pamicogrel, Parecoxib, Robenacoxib, Rofecoxib, Rutecarpine, Tilmacoxib, and Zaltoprofen. Examples of dual COX1/COX2 inhibitors that can be co-administered include without limitation, HP-5000, lornoxicam, ketorolac tromethamine, bromfenac sodium, ATB-346, HP-5000. Examples of dual COX-2/carbonic anhydrase (CA) inhibitors that can be co-administered include without limitation polmacoxib and imrecoxib.

Examples of inhibitors of secreted phospholipase A2, prostaglandin E synthase (PTGES, PGES; Gene ID: 9536) that can be co-administered include without limitation LY3023703, GRC 27864, and compounds described in WO2015158204, WO2013024898, WO2006063466, WO2007059610, WO2007124589, WO2010100249, WO2010034796, WO2010034797, WO2012022793, WO2012076673, WO2012076672, WO2010034798, WO2010034799, WO2012022792, WO2009103778, WO2011048004, WO2012087771, WO2012161965, WO2013118071, WO2013072825, WO2014167444, WO2009138376, WO2011023812, WO2012110860, WO2013153535, WO2009130242, WO2009146696, WO2013186692, WO2015059618, WO2016069376, WO2016069374, WO2009117985, WO2009064250, WO2009064251, WO2009082347, WO2009117987, and WO2008071173. Metformin has further been found to repress the COX2/PGE2/STAT3 axis, and can be co-administered. See, e.g., Tong, et al., Cancer Lett. (2017) 389:23-32; and Liu, et al., Oncotarget. (2016) 7(19):28235-46.

Examples of inhibitors of carbonic anhydrase (e.g., one or more of CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)) that can be co-administered include without limitation acetazolamide, methazolamide, dorzolamide, zonisamide, brinzolamide and dichlorphenamide. A dual COX-2/CA1/CA2 inhibitor that can be co-administered includes CG100649.

Examples of inhibitors of arachidonate 5-lipoxygenase (ALOX5, 5-LOX; NCBI Gene ID: 240) that can be co-administered include without limitation meclofenamate sodium, zileuton.

Examples of inhibitors of soluble epoxide hydrolase 2 (EPHX2, SEH; NCBI Gene ID: 2053) that can be co-administered include without limitation compounds described in WO2015148954. Dual inhibitors of COX-2/SEH that can be co-administered include compounds described in WO2012082647. Dual inhibitors of SEH and fatty acid amide hydrolase (FAAH; NCBI Gene ID: 2166) that can be co-administered include compounds described in WO2017160861.

Examples of inhibitors of mitogen-activated protein kinase kinase kinase 8 (MAP3K8, tumor progression loci-2, TPL2; NCBI Gene ID: 1326) that can be co-administered include without limitation GS-4875, GS-5290, BHM-078 and those described, e.g., in WO2006124944, WO2006124692, WO2014064215, WO2018005435, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42; and Hu, et al., Bioorg Med Chem Lett. (2011) 21(16):4758-61.

### Tumor Oxygenation Agents

In various embodiments, the agent that inhibits binding between CD47 and SIRPα (e.g., magrolimab); and the VEGFA/VEGFR inhibiting agent (e.g., bevacizumab), as described herein, is further combined with an agent that promotes or increases tumor oxygenation or reoxygenation, or prevents or reduces tumor hypoxia. Illustrative agents that can be co-administered include, e.g., Hypoxia inducible factor-1 alpha (HIF-la) inhibitors, such as PT-2977, PT-2385; VEGF inhibitors, such as bevasizumab, IMC-3C5, GNR-011, tanibirumab, LYN-00101, ABT-165; and/or an oxygen carrier protein (e.g., a heme nitric oxide and/or oxygen binding protein (HNOX)), such as OMX-302 and HNOX proteins described in WO 2007/137767, WO 2007/139791, WO 2014/107171, and WO 2016/149562.

### Immunotherapeutic Agents

In various embodiments, the agent that inhibits binding between CD47 and SIRPα (e.g., magrolimab); and the VEGFA/VEGFR inhibiting agent (e.g., bevacizumab), as described herein, is further combined with an immunotherapeutic agent. Example immunotherapeutic agents that can be co-administered include without limitation abagovomab, ABP-980, adecatumumab, afutuzumab, alemtuzumab, altumomab, amatuximab, anatumomab, arcitumomab, bavituximab, bectumomab, bevacizumab biosimilar, bivatuzumab, blinatumomab, brentuximab, cantuzumab, catumaxomab, CC49, cetuximab, citatuzumab, cixutumumab, clivatuzumab, conatumumab, dacetuzumab, dalotuzumab, daratumumab, detumomab, dinutuximab, drozitumab, duligotumab, dusigitumab, ecromeximab, emibetuzumab, ensituximab, ertumaxomab, etaracizumab, farletuzumab, figitumumab, flanvotumab, futuximab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, matuzumab, milatuzumab, minretumomab, mitumomab, moxetumomab, moxetumomab pasudotox, naptumomab, narnatumab, necitumumab, nimotuzumab, nofetumomab, OBI-833, obinutuzumab, ocaratuzumab, ofatumumab, olaratumab, onartuzumab, oportuzumab, oregovomab, panitumumab, parsatuzumab, pasudotox, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, simtuzumab, tacatuzumab, taplitumomab, tenatumomab, teprotumumab, tigatuzumab, tositumomab, trastuzumab, trastuzumab biosimilar, tucotuzumab, ubilituximab, veltuzumab, vorsetuzumab, votumumab, zalutumumab, and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90 (90Y-clivatuzumab), or iodine-131.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein (e.g., in Table B). Example ADCs that can be co-administered include without limitation gemtuzumab, brentuximab, trastuzumab, inotuzumab, glembatumumab, anetumab, mirvetuximab, depatuxizumab, rovalpituzumab, vadastuximab, labetuzumab, sacituzumab, lifastuzumab, indusatumab, polatzumab, pinatuzumab, coltuximab, indatuximab, milatuzumab, rovalpituzumab, ABBV-011, ABBV-2029, ABBV-321, ABBV-647, MLN0264 (anti-GCC, guanylyl cyclase C), T-DM1 (trastuzumab emtansine, Kadcycla); SYD985 (anti-HER2, Duocarmycin), milatuzumab-doxorubicin (hCD74-DOX), DCDT2980S, belantamab mafodotin (GSK2857916), polatuzumab vedotin (RG-7596), SGN-CD70A, SGN-CD19A, inotuzumab ozogamicin (CMC-544), lorvotuzumab mertansine, SAR3419, sacituzumab govetecan (TRODELVY®), isactuzumab govitecan, enfortumab vedotin (ASG-22ME), ASG-15ME, DS-8201 ((trastuzumab deruxtecan), 225Ac-lintuzumab, U3-1402, 177Lu-tetraxetan-tetuloma, tisotumab vedotin, anetumab ravtansine, CX-2009, SAR-566658, W-0101, ABBV-085, gemtuzumab ozogamicin, ABT-414, glembatumumab vedotin (CDX-011), labetuzumab govitecan (IMMU-130), lifastuzumab vedotin, (RG-7599), milatuzumab-doxorubicin (IMMU-110), indatuximab ravtansine (BT-062), pinatuzumab vedotin (RG-7593), SGN-LIV1A, SGN-CD33A, SAR566658, MLN2704, SAR408701, rovalpituzumab tesirine, ABBV-399, AGS-16C3F, ASG-22ME, AGS67E, AMG 172, AMG 595, AGS-15E, BAY1129980, BAY1187982, BAY94-934 (anetumab ravtansine), GSK2857916, Humax-TF-ADC (tisotumab vedotin), IMGN289, IMGN529, IMGN853 (mirvetuximab soravtansine), LOP628, PCA062, MDX-1203, MEDI-547, PF-06263507, PF-06647020, PF-06647263, PF-06664178, PF-06688992, PF-06804103, RG7450, RG7458, RG7598, SAR566658, SGN-CD33A, DS-1602 and DS-7300, DS-6157, DS-6000, TAK-164, MEDI2228, MEDI7247, AMG575. ADCs that can be co-administered are described, e.g., in Lambert, et al., Adv Ther (2017) 34:1015-1035 and in de Goeij, Current Opinion in Immunology (2016) 40:14-23.

Illustrative therapeutic agents (e.g., anticancer or antineoplastic agents) that can be conjugated to the drug-conjugated antibodies, fragments thereof, or antibody mimetics include without limitation monomethyl auristatin E (MMAE), monomethyl auristatin F (MMAF), a calicheamicin, ansamitocin, maytansine or an analog thereof (e.g., mertansine/emtansine (DM1), ravtansine/soravtansine (DM4)), an anthracyline (e.g., doxorubicin, daunorubicin, epirubicin, idarubicin), pyrrolobenzodiazepine (PBD) DNA cross-linking agent SC-DR002 (D6.5), duocarmycin, a microtubule inhibitors (MTI) (e.g., a taxane, a vinca alkaloid, an epothilone), a pyrrolobenzodiazepine (PBD) or dimer thereof, a duocarmycin (A, B1, B2, C1, C2, D, SA, CC-1065), and other anticancer or anti-neoplastic agents described herein.

### Cancer Gene Therapy and Cell Therapy

In various embodiments, the agent that inhibits binding between CD47 and SIRPα (e.g., magrolimab); and the VEGFA/VEGFR inhibiting agent (e.g., bevacizumab), as described herein, is further combined with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

### Cellular Therapies

In various embodiments, the agent that inhibits binding between CD47 and SIRPα (e.g., magrolimab); and the VEGFA/VEGFR inhibiting agent (e.g., bevacizumab), as described herein, is further combined with one or more cellular therapies. Illustrative cellular therapies include without limitation co-administration of one or more of a population of immune cells. In some embodiments, the immune cells are natural killer (NK) cells, NK-T cells, T cells, gamma delta T cells, B-cells, cytokine-induced killer (CIK) cells, macrophage (MAC) cells, tumor infiltrating lymphocytes (TILs) a granulocyte, an innate lymphoid cell, a megakaryocyte, a monocyte, a macrophage, a platelet, a thymocyte, a myeloid cell, and/or dendritic cells (DCs). In some embodiments, the cellular therapy entails a T cell therapy, e.g., co-administering a population of alpha/beta TCR T cells, gamma/delta TCR T cells, regulatory T (Treg) cells and/or TRuC™ T cells. In some embodiments, the cellular therapy entails a NK cell therapy, e.g., co-administering NK-92 cells or JK500 cells. As appropriate, a cellular therapy can entail the co-administration of cells that are autologous, syngeneic or allogeneic to the subject.

In some embodiments, the cellular therapy entails co-administering immune cells engineered to express chimeric antigen receptors (CARs) or T cell receptors (TCRs) TCRs. In particular embodiments, a population of immune cells is engineered to express a CAR, wherein the CAR comprises a tumor antigen-binding domain. In other embodiments, a population of immune cells is engineered to express T cell receptors (TCRs) engineered to target tumor derived peptides presented on the surface of tumor cells. In one embodiment, the immune cell engineered to express chimeric antigen receptors (CARs) or T cell receptors (TCRs) TCRs is a T cell. In another embodiment, the immune cell engineered to express chimeric antigen receptors (CARs) or T cell receptors (TCRs) TCRs is an NK cell.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from CD3 zeta, CD3 gamma, CD3 delta, CD3 epsilon, common FcR gamma (FCERIG), FcR beta (Fc Epsilon Rlb), CD79a, CD79b, Fcgamma RIIa, DAP10, and DAP12 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, the costimulatory domain comprises a functional domain of one or more proteins selected from CD27, CD28, 4-1BB(CD137), OX40, CD30, CD40, PD-1, ICOS, CD2, CD7, LIGHT, NKG2C, lymphocyte function-associated antigen-1 (LFA-1), MYD88, B7-H3, a ligand that specifically binds with CD83, CDS, ICAM-1, GITR, BAFFR, HVEM (LIGHTR), SLAMF7, NKp80 (KLRFI), CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CD1B (NCBI Gene ID: 910), CD1C (NCBI Gene ID: 911), CD1D (NCBI Gene ID: 912), CD1E (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain derived from a protein selected from the alpha, beta or zeta chain of the T-cell receptor, CD28, CD3 epsilon, CD3 delta, CD3 gamma, CD45, CD4, CD5, CD7, CD8 alpha, CD8 beta, CD9, CD11a, CD11b, CD11c, CD11d, CD16, CD18, CD22, CD33, CD37, CD64, CD80, CD86, CD134, CD137, CD154, KIRDS2, OX40, CD2, CD27, ICOS (CD278), 4-1BB(CD137), GITR, CD40, BAFFR, HVEM (LIGHTR), SLAMF7, NKp80 (KLRF1), CD19, CD19a, IL2R beta, IL2R gamma, IL7R alpha, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, ITGB2, ITGB7, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C activating NK cell receptors, an Immunoglobulin protein, BTLA, CD247, CD276 (B7-H3), CD30, CD84, CDS, cytokine receptor, Fc gamma receptor, GADS, ICAM-1, Ig alpha (CD79a), integrins, LAT, a ligand that binds with CD83, LIGHT, MHC class 1 molecule, PAG/Cbp, TNFSF14, a Toll ligand receptor, TRANCE/RANKL, or a fragment, truncation, or a combination thereof.

In some embodiments, the CAR comprises a hinge domain. A hinge domain may be derived from a protein selected from the CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, IgG1, IgG2, IgG3, IgG4, IgA, IgD, IgE, IgM or fragment or combination thereof.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvlll); ganglioside G2 (GD2); ganglioside GD3 (αNeuSAc(2-8)αNeuSAc(2-3)βDGaip(1-4)bDGIcp(1-1)Cer); ganglioside GM3 (αNeuSAc(2-3)βDGalp(1-4)βDGlcp(1-1)Cer); GM-CSF receptor; TNF receptor superfamily member 17 (TNFRSF17, BCMA); B-lymphocyte cell adhesion molecule; Tn antigen ((Tn Ag) or (GaINAcu-Ser/Thr)); prostate-specific membrane antigen (PSMA); Receptor tyrosine kinase-like orphan receptor 1 (RORI); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); HLA class I antigen A-2 alpha; HLA antigen; Lewis(Y)antigen; CD24; Platelet-derived growth factor receptor beta (PDGFR-beta); Stage-specific embryonic antigen-4 (SSEA-4); CD20; delta like 3 (DLL3); Folate receptor alpha; Folate receptor beta, GDNF alpha 4 receptor, Receptor tyrosine-protein kinase, ERBB2 (Her2/neu); Mucin 1, cell surface associated (MUC1); APRIL receptor; ADP ribosyl cyclase-1; Ephb4 tyrosine kinase receptor, DCAMKL1 serine threonine kinase, Aspartate beta-hydroxylase, epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); ephrin type-A receptor 3 (EphA3), Fucosyl GM1; sialyl Lewis adhesion molecule (sLe); transglutaminase 5 (TGS5); high molecular weight-melanomaassociatedantigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAPI); claudin 6 (CLDN6); thyroid stimulating hormone receptor (TSHR); G protein-coupled receptor class C group 5, member D (GPRCSD); IL-15 receptor (IL-15); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma associated antigen 1 (MAGE-A1); Melanoma associated antigen 3 (MAGE-A3); Melanoma associated antigen 4 (MAGE-A4); T cell receptor beta 2 chain C; ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin-A1; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1(CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES I); lymphocyte-specific protein tyrosine kinase (LCK); A kinase anchor protein 4 (AKAP-4); Peptidoglycan recognition protein, synovial sarcoma, X breakpoint 2 (SSX2); Receptor for Advanced Glycation Endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-2 (GPC2); Glypican-3 (GPC3); Fc receptor-like 5 (FCRL5); and immunoglobulin lambda-like polypeptide 1 (IGLL1). In some embodiments, the target is an epitope of the tumor associated antigen presented in an MHC.

In some embodiments, the tumor antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HERV-K, HIV-1 envelope glycoprotein gp120, HIV-1 envelope glycoprotein gp41, HLA-DR, HLA class I antigen alpha G, HM1.24, K-Ras GTPase, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine e receptor, folate binding protein, gp100, hepatitis B surface antigen, Epstein-Barr nuclear antigen 1, Latent membrane protein 1, Secreted protein BARF1, P2X7 purinoceptor, Syndecan-1, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

Examples of cell therapies include without limitation: AMG-119, Algenpantucel-L, ALOFISEL®, Sipuleucel-T, (BPX-501) rivogenlecleucel U.S. Pat. No. 9,089,520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, SNK-01, NEXI-001, CryoStim, AlloStim, lentiviral transduced huCART-meso cells, CART-22 cells, EGFRt/19-28z/4-1BBL CAR T cells, autologous 4H11-28z/fIL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502, CMD-601, CMD-602, CSG-005, LAAP T-cell therapy, PD-1 knockout T cell therapy (esophageal cancer/NSCLC), anti-MUC1 CAR T-cell therapy (esophageal cancer/NSCLC), anti-MUC1 CAR T-cell therapy+PD-1 knockout T cell therapy (esophageal cancer/NSCLC), anti-KRAS G12D mTCR PBL, anti-CD123 CAR T-cell therapy, anti-mutated neoantigen TCR T-cell therapy, tumor lysate/MUC1/survivin PepTivator-loaded dendritic cell vaccine, autologous dendritic cell vaccine (metastatic malignant melanoma, intradermal/intravenous), anti-LeY-scFv-CD28-zeta CAR T-cells, PRGN-3005, iC9-GD2-CAR-IL-15 T-cells, HSC-100, ATL-DC-101, MIDRIX4-LUNG, MIDRIXNEO, FCR-001, PLX stem cell therapy, MDR-101, GeniusVac-Mel4, ilixadencel, allogeneic mesenchymal stem cell therapy, romyelocel L, CYNK-001, ProTrans, ECT-100, MSCTRAIL, dilanubicel, FT-516, ASTVAC-2, E-CEL UVEC, CK-0801, allogenic alpha/beta CD3+ T cell and CD19+ B cell depleted stem cells (hematologic diseases, TBX-1400, HLCN-061, umbilical cord derived Hu-PHEC cells (hematological malignancies/aplastic anemia), AP-011, apceth-201, apceth-301, SENTI-101, stem cell therapy (pancreatic cancer), ICOVIR15-cBiTE, CD33HSC/CD33 CAR-T, PLX-Immune, SUBCUVAX, CRISPR allogeneic gamma-delta T-cell based gene therapy (cancer), ex vivo CRISPR allogeneic healthy donor NK-cell based gene therapy (cancer), ex-vivo allogeneic induced pluripotent stem cell-derived NK-cell based gene therapy (solid tumor), and anti-CD20 CAR T-cell therapy (non-Hodgkin's lymphoma).

### Additional Agents for Targeting Tumors

Additional agents for targeting tumors include without limitation: Alpha-fetoprotein modulators, such as ET-1402, and AFP-TCR; Anthrax toxin receptor 1 modulator, such as anti-TEM8 CAR T-cell therapy; TNF receptor superfamily member 17 (TNFRSF17, BCMA), such as bb-2121 (ide-cel), bb-21217, JCARH125, UCART-BCMA, ET-140, MCM-998, LCAR-B38M, CART-BCMA, SEA-BCMA, BB212, ET-140, P-BCMA-101, AUTO-2 (APRIL-CAR), JNJ-68284528; Anti-CLL-1 antibodies, (see, for example, PCT/US2017/025573); Anti-PD-LI-CAR tank cell therapy, such as KD-045; Anti-PD-L1 t-haNK, such as PD-L1 t-haNK; anti-CD45 antibodies, such as 131I-BC8 (lomab-B); anti-HER3 antibodies, such as LJM716, GSK2849330; APRIL receptor modulator, such as anti-BCMA CAR T-cell therapy, Descartes-011; ADP ribosyl cyclase-1/APRIL receptor modulator, such as dual anti-BCMA/anti-CD38 CAR T-cell therapy; CART-ddBCMA; B7 homolog 6, such as CAR-NKp30 and CAR-B7H6; B-lymphocyte antigen CD19, such as TBI-1501, CTL-119 huCART-19 T cells,l iso-cel, JCAR-015 U.S. Pat. No. 7,446,190, JCAR-014, JCAR-017, (WO2016196388, WO2016033570, WO2015157386), axicabtagene ciloleucel (KTE-C19, Yescarta®), KTE-X19, U.S. Pat. Nos. 7,741,465, 6,319,494, UCART-19, EBV-CTL, T tisagenlecleucel-T (CTL019), WO2012079000, WO2017049166, CD19CAR-CD28-CD3zeta-EGFRt-expressing T cells, CD19/4-1BBL armored CAR T cell therapy, C-CAR-011, CIK-CAR.CD19, CD19CAR-28-zeta T cells, PCAR-019, MatchCART, DSCAR-01, IM19 CAR-T, TC-110; anti-CD19 CAR T-cell therapy (B-cell acute lymphoblastic leukemia, Universiti Kebangsaan Malaysia); anti-CD19 CAR T-cell therapy (acute lymphoblastic leukemia/Non-Hodgkin's lymphoma, University Hospital Heidelberg), anti-CD19 CAR T-cell therapy (silenced IL-6 expression, cancer, Shanghai Unicar-Therapy Bio-medicine Technology), MB-CART2019.1 (CD19/CD20), GC-197 (CD19/CD7), CLIC-1901, ET-019003, anti-CD19-STAR-T cells, AVA-001, BCMA-CD19 cCAR (CD19/APRIL), ICG-134, ICG-132 (CD19/CD20), CTA-101, WZTL-002, dual anti-CD19/anti-CD20 CAR T-cells (chronic lymphocytic leukemia/B-cell lymphomas), HY-001, ET-019002, YTB-323, GC-012 (CD19/APRIL), GC-022 (CD19/CD22), CD19CAR-CD28-CD3zeta-EGFRt-expressing Tn/mem; UCAR-011, ICTCAR-014, GC-007F, PTG-01, CC-97540; allogeneic anti-CD19 CART cells, such as GC-007G; APRIL receptor modulator; SLAM family member 7 modulator, BCMA-CS1 cCAR; autologous dendritic cell tumor antigen (ADCTA), such as ADCTA-SSI-G; B-lymphocyte antigen CD20, such as ACTR707 ATTCK-20, PBCAR-20A; allogenic T cells expressing CD20 CAR, such as LB-1905; B-lymphocyte antigen CD19/B-lymphocyte antigen 22, such as TC-310; B-lymphocyte antigen 22 cell adhesion, such as UCART-22, JCAR-018 WO2016090190; NY-ESO-1 modulators, such as GSK-3377794, TBI-1301, GSK3537142; Carbonic anhydrase, such as DC-Ad-GMCAIX; Caspase 9 suicide gene, such as CaspaCIDe DLI, BPX-501; CCR5, such as SB-728; CCR5 gene inhibitor/TAT gene/TRIM5 gene stimulator, such as lentivirus vector CCR5 shRNA/TRIM5alpha/TAR decoy-transduced autologous CD34-positive hematopoietic progenitor cells; CDw123, such as MB-102, IM-23, JEZ-567, UCART-123; CD4, such as ICG-122; CD5 modulators, such as CD5.28z CART cells; Anti-CD22, such as anti-CD22 CART; Anti-CD30, such as TT-11; Dual anti-CD33/anti-CLL1, such as LB-1910; CD40 ligand, such as BPX-201, MEDI5083; CD56, such as allogeneic CD56-positive CD3-negative natural killer cells (myeloid malignancies); CD19/CD7 modulator, such as GC-197; T-cell antigen CD7 modulator, such as anti-CD7 CAR T-cell therapy (CD7-positive hematological malignancies); CD123 modulator, such as UniCAR02-T-CD123; Anti-CD276, such as anti-CD276 CART; CEACAM protein 5 modulators, such as MG7-CART; Claudin 6, such as CSG-002; Claudin 18.2, such as LB-1904; Chlorotoxin, such as CLTX-CART; EBV targeted, such as CMD-003; MUC16EGFR, such as autologous 4H11-28z/fIL-12/EFGRt T cell; Endonuclease, such as PGN-514, PGN-201; Epstein-Barr virus specific T-lymphocytes, such as TT-10; Epstein-Barr nuclear antigen 1/Latent membrane protein 1/Secreted protein BARF1 modulator, such as TT-10X; Erbb2, such as CST-102, CIDeCAR; Ganglioside (GD2), such as 4SCAR-GD2; Gamma delta T cells, such as ICS-200; folate hydrolase 1 (FOLH1, Glutamate carboxypeptidase II, PSMA; NCBI Gene ID: 2346), such as CIK-CAR.PSMA, CART-PSMA-TGFβRDN, P-PSMA-101; Glypican-3(GPC3), such as TT-16, GLYCAR; Hemoglobin, such as PGN-236; Hepatocyte growth factor receptor, such as anti-cMet RNA CAR T; HLA class I antigen A-2 alpha modulator, such as FH-MCVA2TCR; HLA class I antigen A-2 alpha/Melanoma associated antigen 4 modulator, such as ADP-A2M4CD8; HLA antigen modulator, such as FIT-001, NeoTCR-P1; Human papillomavirus E7 protein, such as KITE-439 (see, for example, PCT/US2015/033129); ICAM-1 modulator, such as AIC-100; Immunoglobulin gamma Fc receptor III, such as ACTR087; IL-12, such as DC-RTS-IL-12; IL-12 agonist/mucin 16, such as JCAR-020; IL-13 alpha 2, such as MB-101; IL-15 receptor agonist, such as PRGN-3006, ALT-803; interleukin-15/Fc fusion protein (e.g., XmAb24306); recombinant interleukin-15 (e.g., AM0015, NIZ-985); pegylated IL-15 (e.g., NKTR-255); IL-2, such as CST-101; Interferon alpha ligand, such as autologous tumor cell vaccine+systemic CpG-B+IFN-alpha (cancer); K-Ras GTPase, such as anti-KRAS G12V mTCR cell therapy; Neural cell adhesion molecule L1 L1CAM (CD171), such as JCAR-023; Latent membrane protein 1/Latent membrane protein 2, such as Ad5f35-LMPd1-2-transduced autologous dendritic cells; MART-1 melanoma antigen modulator, such as MART-1 F5 TCR engineered PBMC; Melanoma associated antigen 10, such as MAGE-A10C796T MAGE-A10 TCR; Melanoma associated antigen 3/Melanoma associated antigen 6 (MAGE A3/A6) such as KITE-718 (see, for example, PCT/US2013/059608); Mesothelin, such as CSG-MESO, TC-210; Mucin 1 modulator, such as ICTCAR-052, Tn MUC-1 CAR-T, ICTCAR-053; Anti-MICA/MICB, such as CYAD-02; NKG2D, such as NKR-2; Ntrkr1 tyrosine kinase receptor, such as JCAR-024; PRAMET cell receptor, such as BPX-701; Prostate stem cell antigen modulator, such as MB-105; Roundabout homolog 1 modulator, such as ATCG-427; Peptidoglycan recognition protein modulator, such as Tag-7 gene modified autologous tumor cell vaccine; PSMA, such as PSMA-CAR T-cell therapy (lentiviral vector, castrate-resistant prostate cancer); SLAM family member 7 modulator, such as IC9-Luc90-CD828Z; TGF beta receptor modulator, such as DNR.NPC T-cells; T-lymphocyte, such as TT-12; T-lymphocyte stimulator, such as ATL-001; TSH receptor modulator, such as ICTCAR-051; Tumor infiltrating lymphocytes, such as LN-144, LN-145; and/or Wilms tumor protein, such as JTCR-016, WT1-CTL, ASP-7517.

### MCL1 Apoptosis Regulator, BCL2 Family Member (MCL1) Inhibitors

In various embodiments, an anti-CD47 agent or an anti-SIRPα agent as described herein, is combined with an inhibitor of MCL1 apoptosis regulator, BCL2 family member (MCL1, TM; EAT; MCL1L; MCL1S; Mel-1; BCL2L3; MCL1-ES; bcl2-L-3; mcl1/EAT; NCBI Gene ID: 4170). Examples of MCL1 inhibitors include AMG-176, AMG-397, S-64315, and AZD-5991, 483-LM, A-1210477, UMI-77, JKY-5-037, and those described in WO2018183418, WO2016033486, WO2019222112 and WO2017147410.

### Cytokine Inducible SH2 Containing Protein (CISH) Inhibitors

In various embodiments, the agent that inhibits binding between CD47 and SIRPα (e.g., magrolimab); and the VEGFA/VEGFR inhibiting agent (e.g., bevacizumab), as described herein, is further combined with an inhibitor of cytokine inducible SH2 containing protein (CISH; CIS; G18; SOCS; CIS-1; BACTS2; NCBI Gene ID: 1154). Examples of CISH inhibitors include those described in WO2017100861, WO2018075664 and WO2019213610.

### Gene Editors

In various embodiments, the agent that inhibits binding between CD47 and SIRPα (e.g., magrolimab); and the VEGFA/VEGFR inhibiting agent (e.g., bevacizumab), as described herein, is further combined with gene editor. Illustrative gene editing system that can be co-administered include without limitation a CRISPR/Cas9 system, a zinc finger nuclease system, a TALEN system, a homing endonucleases system (e.g., an ARCUS), and a homing meganuclease system.

Other Drugs with Unspecified Targets

In various embodiments, the agent that inhibits binding between CD47 and SIRPα (e.g., magrolimab) and the VEGFA/VEGFR inhibiting agent (e.g., bevacizumab), as described herein, is further combined with human immunoglobulin (10% liquid formulation), Cuvitru (human immunoglobulin (20% solution), levofolinate disodium, IMSA-101, BMS-986288, IMUNO BGC Moreau RJ, R-OKY-034F, GP-2250, AR-23, calcium levofolinate, porfimer sodium, RG6160, ABBV-155, CC-99282, polifeprosan 20 with carmustine, Veregen, gadoxetate disodium, gadobutrol, gadoterate meglumine, gadoteridol, 99mTc-sestamibi, pomalidomide, pacibanil, and/or valrubicin.

### Exemplified Combination Therapies

In various embodiments, the agent that inhibits binding between CD47 and SIRPα (e.g., magrolimab) and the VEGFA/VEGFR inhibiting agent (e.g., bevacizumab), as described herein, is further combined with standard of care regimens for treating colorectal cancer.

Therapeutic agents used to treat CRC include bevacizumab, capecitabine, cetuximab, fluorouracil, irinotecan, leucovorin, oxaliplatin, panitumumab, ziv-aflibercept, and any combinations thereof. In some embodiments therapeutic agents used to treat CRC include bevacizumab (AVASTIN®), leucovorin, 5-FU, oxaliplatin (FOLFOX), pembrolizumab (KEYTRUDA®), FOLFIRI, regorafenib (STIVARGA®), aflibercept (ZALTRAP®), cetuximab (ERBITUX®), Lonsurf (ORCANTAS®), XELOX, FOLFOXIRI, or a combination thereof. In some embodiments therapeutic agents used to treat CRC include bevacizumab+leucovorin+5-FU+oxaliplatin (FOLFOX), bevacizumab+FOLFIRI, bevacizumab+FOLFOX, aflibercept+FOLFIRI, cetuximab+FOLFIRI, bevacizumab+XELOX, and bevacizumab+FOLFOXIRI. In some embodiments therapeutic agents used to treat CRC include binimetinib+encorafenib+cetuximab, trametinib+dabrafenib+panitumumab, trastuzumab+pertuzumab, napabucasin+FOLFIRI+bevacizumab, nivolumab+ipilimumab.

### Additional Exemplified Combination Therapies

In some embodiments, the agent that inhibits binding between CD47 and SIRPα (e.g., magrolimab) and the VEGFA/VEGFR inhibiting agent (e.g., bevacizumab), as described herein, are co-administered with one or more therapeutic agents selected from a PI3K inhibitor, a FLT3R agonist, a PD-1 antagonist, a PD-L1 antagonist, an MCL1 inhibitor, a CCR8 binding agent, an HPK1 antagonist, a DGKα inhibitor, a CISH inhibitor, a PARP-7 inhibitor, a Cbl-b inhibitor, a KRAS inhibitor (e.g., a KRAS G12C or G12D inhibitor), a KRAS degrader, a beta-catenin degrader, a helios degrader, a CD73 inhibitor, an adenosine receptor antagonist, a TIGIT antagonist, a TREM1 binding agent, a TREM2 binding agent, a CD137 agonist, a GITR binding agent, an OX40 binding agent, and a CAR-T cell therapy.

In some embodiments, the agent that inhibits binding between CD47 and SIRPα (e.g., magrolimab) and the VEGFA/VEGFR inhibiting agent (e.g., bevacizumab), as described herein, are co-administered with one or more therapeutic agents selected from a PI3KS inhibitor (e.g., idealisib), a FLT3L-Fc fusion protein (e.g., GS-3583), an anti-PD-1 antibody (pembrolizumab, nivolumab, zimberelimab), a small molecule PD-L1 inhibitor (e.g., GS-4224), an anti-PD-L1 antibody (e.g., atezolizumab, avelumab), a small molecule MCL1 inhibitor (e.g., GS-9716), a small molecule HPK1 inhibitor (e.g., GS-6451), a HPK1 degrader (PROTAC; e.g., ARV-766), a small molecule DGKα inhibitor, a small molecule CD73 inhibitor (e.g., quemliclustat (AB680)), an anti-CD73 antibody (e.g., oleclumab), a dual A2a/A2b adenosine receptor antagonist (e.g., etrumadenant (AB928)), an anti-TIGIT antibody (e.g., tiragolumab, vibostolimab, domvanalimab, AB308), an anti-TREM1 antibody (e.g., PY159), an anti-TREM2 antibody (e.g., PY314), a CD137 agonist (e.g., AGEN-2373), a GITR/OX40 binding agent (e.g., AGEN-1223) and a CAR-T cell therapy (e.g., axicabtagene ciloleucel, brexucabtagene autoleucel, tisagenlecleucel).

In some embodiments, the agent that inhibits binding between CD47 and SIRPα (e.g., magrolimab) and the VEGFA/VEGFR inhibiting agent (e.g., bevacizumab), as described herein, are co-administered with one or more therapeutic agents selected from idealisib, GS-3583, zimberelimab, GS-4224, GS-9716, GS-6451, quemliclustat (AB680), etrumadenant (AB928), domvanalimab, AB308, PY159, PY314, AGEN-1223, AGEN-2373, axicabtagene ciloleucel and brexucabtagene autoleucel.

5. Dosing and Scheduling

The methods described herein include administration of a therapeutically effective dose of compositions, e.g., a therapeutically effective dose of an agent that inhibits binding between CD47 and SIRPα and a therapeutically effective dose of a VEGFA/VEGFR inhibiting agent.

Compositions are administered to a patient in an amount sufficient to substantially ablate targeted cells, as described above. An amount adequate to accomplish this is defined as a “therapeutically effective dose,” which may provide for an improvement in overall survival rates. The term “therapeutically effective amount” is an amount that is effective to ameliorate a symptom of a disease (e.g., a cancer as described herein). A therapeutically effective amount can be a “prophylactically effective amount” as prophylaxis can be considered therapy. Single or multiple administrations of the compositions may be administered depending on the dosage and frequency as needed and tolerated by the patient. The particular dose used for a treatment will depend upon the medical condition and history of the mammal, as well as other factors such as age, weight, gender, administration route, efficiency, etc.

In some embodiments, combined therapeutic amounts of an agent that inhibits binding between CD47 and SIRPα; and a VEGFA/VEGFR inhibiting agent, as described herein, optionally, with one or more additional therapeutic agents, as described herein, can (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer or myeloproliferative disease. In some embodiments, combined therapeutic amounts of an agent that inhibits binding between CD47 and SIRPα; and a VEGFA/VEGFR inhibiting agent, as described herein, optionally, with one or more additional therapeutic agents, as described herein, can (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

An “increased” or “enhanced” amount (e.g., with respect to cancer cell proliferation or expansion, antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein. In various embodiments, tumor burden is determined using linear dimensional methods (e.g. Response Evaluation Criteria in Solid Tumors (RECIST) v1.1 (Eisenhauer, et al., Eur J Cancer. (2009) 45(2):228-47). In various embodiments, tumor burden is determined using volumetric analysis (e.g., positron emission tomography (PET)/computed tomography (CT) scan). See, e.g., Paydary, et al., Mol Imaging Biol. (2019) 21(1):1-10; Li, et al., AJR Am JRoentgenol. (2021) 217(6):1433-1443; and Kerner, et al., EJNMMI Res. (2016) December; 6(1):33.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence or recurrence of a tumor, e.g., a relapse after remission.

Effective doses of the combined agents for the treatment of cancer vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as non-human primates, rabbits, mice, rats, etc., and the like. Treatment dosages can be titrated to optimize safety and efficacy.

A therapeutically effective dose of an anti-CD47 antibody can depend on the specific agent used, but is usually about 10 mg/kg body weight or more (e.g., about 10 mg/kg or more, about 15 mg/kg or more, 20 mg/kg or more, about 25 mg/kg or more, about 30 mg/kg or more, about 35 mg/kg or more, about 40 mg/kg or more, about 45 mg/kg or more, about 50 mg/kg or more, or about 55 mg/kg or more, or about 60 mg/kg or more, or about 65 mg/kg or more, or about 70 mg/kg or more), or from about 10 mg/kg, from about 15 mg/kg to about 70 mg/kg (e.g., from about 10 mg/kg to about 67.5 mg/kg, or from about 10 mg/kg, from about 15 mg/kg to about 60 mg/kg).

In some embodiments, the therapeutically effective dose of the anti-CD47 antibody is 10, 15, 20, 25, 30, 35, 40, 45, 50, 55, 60, 65, or 67.5 mg/kg. In some embodiments, the therapeutically effective dose of the anti-CD47 antibody is 10 to 60 mg/kg. In some embodiments, the therapeutically effective dose of the anti-CD47 antibody is 10 to 67.5 mg/kg. In some embodiments, the anti-CD47 antibody is administered at a dose of at least 10-30, 20-30, 15-60, 30-60, 10, 15, 20, 30, 40, 45, 50, or 60 mg of antibody per kg of body weight.

A therapeutic dose of an anti-CD47 antibody can be a flat dose. For example, a flat dose can be given irrespective of a particular subject's weight. Alternatively, a flat dose can be given based on a particular subject's weight falling within a particular weight range, e.g., a first range of less than or equal to 100 kg; or a second range of greater than 100 kg. A flat dose can be, e.g., 1000-5000, 2000-4000, 2000-3500, 2400-3500, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 mg, or an interim number of mg thereof.

Methods can include a step of administering a primer agent to subject, followed by a step of administering a therapeutically effective dose of an anti-CD47 to the subject. In some embodiments, the step of administering a therapeutically effective dose is performed after at least about 3 days (e.g., at least about 4 days, at least about 5 days, at least about 6 days, at least about 7 days, at least about 8 days, at least about 9 days, or at least about 10 days) after beginning the administration of a primer agent. This period of time is, for example, sufficient to provide for enhanced reticulocyte production by the individual. In some embodiments, the anti-CD47 agent is an isolated anti-CD47 antibody.

The administration of a therapeutically effective dose of an anti-CD47 can be achieved in a number of different ways. In some cases, two or more therapeutically effective doses are administered after a primer agent is administered. Suitable administration of a therapeutically effective dose can entail administration of a single dose, or can entail administration of doses daily, semi-weekly, weekly, once every two weeks, once a month, annually, etc. In some cases, a therapeutically effective dose is administered as two or more doses of escalating concentration (i.e., increasing doses), where (i) all of the doses are therapeutic doses, or where (ii) a sub-therapeutic dose (or two or more sub-therapeutic doses) is initially given and therapeutic doses are achieved by said escalation. As one non-limiting example to illustrate escalating concentration (i.e., increasing doses), a therapeutically effective dose can be administered weekly, beginning with a sub-therapeutic dose (e.g., a dose of less than 10 mg/kg, e.g., 5 mg/kg, 4 mg/kg, 3 mg/kg, 2 mg/kg or 1 mg/kg), and each subsequent dose can be increased by a particular increment (e.g., by 5 mg/kg, by 10 mg/kg, by 15 mg/kg), or by variable increments, until a therapeutic dose (e.g., 15 mg/kg, 30 mg/kg, 45 mg/kg, 60 mg/kg) is reached, at which point administration may cease or may continue with one or more additional therapeutic doses (e.g., continued therapeutic doses or escalated therapeutic doses, e.g., doses of 15 mg/kg, 30 mg/kg, 45 mg/kg, 60 mg/kg). As another non-limiting example to illustrate escalating concentration (i.e., increasing doses), a therapeutically effective dose can be administered weekly, beginning with one or more relatively lower therapeutic doses (e.g., a dose of 10 mg/kg, 15 mg/kg or 30 mg/kg), and each subsequent dose can be increased by a particular increment (e.g., by 10 mg/kg or 15 mg/kg), or by variable increments, until a relatively higher therapeutic dose (e.g., 30 mg/kg, 45 mg/kg, 60 mg/kg, 100 mg/kg, etc.) is reached, at which point administration may cease or may continue (e.g., one or more continued or escalated therapeutic doses, e.g., doses of 30 mg/kg, 45 mg/kg, 60 mg/kg, 100 mg/kg, etc.). In various embodiments, relatively lower therapeutic doses are administered more often (e.g., two or more doses of 15 mg/kg administered weekly (Q1W) or two or more doses of 30 mg/kg administered every two weeks (Q2W)), and relatively higher therapeutic doses are administered less often (e.g., two or more doses of 45 mg/kg administered every 3 weeks (Q3W) or two or more doses of 60 mg/kg administered monthly or every 4 weeks (Q4W)). In some embodiments, administration of a therapeutically effective dose can be a continuous infusion and the dose can altered (e.g., escalated) over time.

The dose needed to achieve and/or maintain a particular serum level of the administered composition is proportional to the amount of time between doses and inversely proportional to the number of doses administered. Thus, as the frequency of dosing increases, the needed dose decreases. The optimization of dosing strategies will be readily understood and practiced by one of ordinary skill in the art. An exemplary treatment regime entails administration once every two weeks or once a month or once every 3 to 6 months. Therapeutic entities described herein are usually administered on multiple occasions. Intervals between single dosages can be weekly, monthly or yearly. Intervals can also be irregular as indicated by measuring blood levels of the therapeutic entity in the patient. Alternatively, therapeutic entities described herein can be administered as a sustained release formulation, in which case less frequent administration is used. Dosage and frequency vary depending on the half-life of the polypeptide in the patient. In some embodiments, the interval between each single dose is a week. In some embodiments, the interval between each single dose is two weeks. In some embodiments, the interval between each single dose is three weeks. In some embodiments, the interval between each single dose is four weeks. In some embodiments, the interval between each single dose of anti-CD47 antibody is a week. In some embodiments, the interval between each single dose of anti-CD47 antibody is two weeks. In some embodiments, the interval between each single dose of anti-CD47 antibody is three weeks. In some embodiments, the interval between each single dose of anti-CD47 antibody is four weeks. In some embodiments, the interval between each single dose of magrolimab is a week. In some embodiments, the interval between each single dose of magrolimab is two weeks. In some embodiments, the interval between each single dose of magrolimab is three weeks. In some embodiments, the interval between each single dose of magrolimab is four weeks.

A “maintenance dose” is a dose intended to be a therapeutically effective dose. For example, in experiments to determine the therapeutically effective dose, multiple different maintenance doses may be administered to different subjects. As such, some of the maintenance doses may be therapeutically effective doses and others may be sub-therapeutic doses.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes used until progression of the disease is reduced or terminated, and preferably until the patient shows partial or complete amelioration of symptoms of disease. Thereafter, the patent can be administered a prophylactic regime.

The term “priming dose” or as used herein refers to a dose of an anti-CD47 antibody that primes a subject for administration of a therapeutically effective dose of anti-CD47 antibody such that the therapeutically effective dose does not result in a severe loss of RBCs (reduced hematocrit or reduced hemoglobin). The specific appropriate priming dose of an anti-CD47 antibody can vary depending on the nature of the agent used and on numerous subject-specific factors (e.g., age, weight, etc.). Examples of suitable priming doses of an anti-CD47 antibody include from about 0.5 mg/kg to about 5 mg/kg, from about 0.5 mg/kg to about 4 mg/kg, from about 0.5 mg/kg to about 3 mg/kg, from about 1 mg/kg to about 5 mg/kg, from about 1 mg/kg to about 4 mg/kg, from about 1 mg/kg to about 3 mg/kg, about 1 mg/kg, about 2 mg/kg, about 3 mg/kg, about 4 mg/kg, about 5 mg/kg. In some embodiments, the priming dose is preferably 1 mg/kg.

In some embodiments of the methods described herein, the anti-CD47 antibody is administered to the subject as a priming dose ranging from about 0.5 mg to about 10 mg, e.g., from about 0.5 to about 5 mg/kg of antibody, optionally, 4 mg/kg, 3 mg/kg, 2 mg/kg, or 1 mg/kg of antibody. In some embodiments, the anti-CD47 antibody is administered to the subject as a therapeutic dose ranging from about 20 to about 67.5 mg/kg of antibody, optionally from 15 to 60 mg/kg of antibody, optionally from 30 to 60 mg/kg of antibody, optionally 15 mg/kg of antibody, 20 mg/kg of antibody, 30 mg/kg of antibody, 45 mg/kg of antibody, 60 mg/kg of antibody, or 67.5 mg/kg of antibody.

A priming dose of an anti-CD47 antibody can be a flat priming dose. For example, a flat priming dose can be given irrespective of a particular subject's weight. Alternatively, a flat priming dose can be given based on a particular subject's weight falling within a particular weight range, e.g., a first range of less than or equal to 100 kg; or a second range of greater than 100 kg. A flat priming dose can be, e.g., 10-200, 50-100, 80-800, 80-400, 80-200, 70-90, 75-85, 10, 20, 30, 40, 50, 60, 70, 80, 90, 100, 110, 120, 130, 140, 150, 160, 170, 180, 190, 200, 240, 300, 320, 400, 500, 600, 700 or 800 mg, or an interim number of mg thereof.

In some embodiments, an effective priming dose of magrolimab is provided, where the effective priming dose for a human is around about 1 mg/kg, e.g., from at least about 0.5 mg/kg up to not more than about 5 mg/kg; from at least about 0.75 mg/kg up to not more than about 1.25 mg/kg; from at least about 0.95 mg/kg up to not more than about 1.05 mg/kg; and may be around about 1 mg/kg.

In some embodiments, an initial dose of a CD47 or SIRPα binding agent is infused over a period of at least about 2 hours, at least about 2.5 hours, at least about 3 hours, at least about 3.5 hours, at least about 4 hours, at least about 4.5 hours, at least about 5 hours, at least about 6 hours or more. In some embodiments an initial dose is infused over a period of time from about 2.5 hours to about 6 hours; for example, from about 3 hours to about 4 hours. In some such embodiments, the dose of agent in the infusate is from about 0.05 mg/ml to about 0.5 mg/ml; for example, from about 0.1 mg/ml to about 0.25 mg/ml.

In other embodiments, an initial dose of a CD47 or SIRPα binding agent, e.g. a priming dose, is administered by continuous fusion, e.g., as an osmotic pump, delivery patch, etc., where the dose is administered over a period of at least about 6 hours, at least about 12 hours, at least about 24 hours, at least about 2 days, at least about 3 days. Many such systems are known in the art. For example, DUROS technology, provides a bi-compartment system separated by a piston. One of the compartments consists of osmotic engine specifically formulated with an excess of solid NaCl, such that it remains present throughout the delivery period and results in a constant osmotic gradient. It also consists of a semi permeable membrane on one end through which water is drawn into the osmotic engine and establishes a large and constant osmotic gradient between the tissue water and the osmotic engine. Other compartment consists of a drug solution with an orifice from which the drug is released due to the osmotic gradient. This helps to provide site specific and systemic drug delivery when implanted in humans. The preferred site of implantation is subcutaneous placement in the inside of the upper arm.

Following administration of the priming agent, and allowing a period of time effective for an increase in reticulocyte production, a therapeutic dose of an anti-CD47 or anti-SIRPα agent is administered. The therapeutic dose can be administered in number of different ways. In some embodiments, two or more therapeutically effective doses are administered after a primer agent is administered, e.g., in a weekly dosing schedule. In some embodiments a therapeutically effective dose of an anti-CD47 agent is administered as two or more doses of escalating concentration, in others the doses are equivalent. There is reduced hemagglutination after the priming dose.

A therapeutically effective dose of an anti-SIRPα antibody can depend on the specific agent used, but is usually about 10 mg or more, e.g., about 30 mg, 50 mg, 100 mg, 200 mg, 400 mg or 800 mg, or more. Multiple administrations of an anti-SIRPα antibody, e.g., without Fc effector function, can be performed over an extended period of time, e.g., over 1, 2, 3, 4, 5, 6, 7, 8, 9, 10, 11, 12 months, at regular intervals, e.g., every 2 weeks (Q2W), every 3 weeks (Q3W), every 4 weeks (Q4W).

With respect to dosing of the VEGFA/VEGFR inhibiting agent (e.g., bevacizumab), in some embodiments, the bevacizumab is administered at one or more doses in the range of 5 mg/kg to 15 mg/kg, e.g., 5 mg/kg to 7.5 mg/kg or 7.5 mg/kg to 15 mg/kg. In some embodiments, the VEGFA/VEGFR inhibiting agent (e.g., bevacizumab) is administered at one or more doses of 5 mg/kg.

In some embodiments, the magrolimab is first administered at a priming dose of 1 mg/kg, then administered at one or more therapeutic doses of 30 mg/kg, followed by administration of one or more therapeutic doses of 60 mg/kg. In some embodiments, the magrolimab is first administered at a priming dose of 1 mg/kg, then administered at one or more therapeutic doses of 20 mg/kg, followed by administration of one or more therapeutic doses of 45 mg/kg. In some embodiments, the magrolimab is first administered at a priming dose of 1 mg/kg, then administered at one or more therapeutic doses of 15 mg/kg, followed by administration of one or more therapeutic doses of 30 mg/kg.

In some embodiments, the magrolimab and the bevacizumab are administered for first, second and third 28-day cycles, wherein:
a) for the first 28-day cycle, magrolimab is administered at a dose of 1 mg/kg on day 1 and at a dose of 30 mg/kg weekly (QW) beginning on day 8; and bevacizumab is administered at a dose of 5 mg/kg on days 1 and 15;
b) for the second 28-day cycle, magrolimab is administered at a dose of 30 mg/kg weekly (QW); and bevacizumab is administered at a dose of 5 mg/kg on days 1 and 15; and
c) for the third 28-day cycle, magrolimab is administered at a dose of 30 mg/kg Q2W; and bevacizumab is administered at a dose of 5 mg/kg on days 1 and 15.

In some embodiments, the magrolimab and the bevacizumab are administered for first, second and third 28-day cycles, wherein:
a) for the first 28-day cycle, magrolimab is administered at a dose of 1 mg/kg on day 1 and at a dose of 20 mg/kg weekly (QW) beginning on day 8; and bevacizumab is administered at a dose of 5 mg/kg on days 1 and 15;
b) for the second 28-day cycle, magrolimab is administered at a dose of 20 mg/kg weekly (QW); and bevacizumab is administered at a dose of 5 mg/kg on days 1 and 15; and
c) for the third 28-day cycle, magrolimab is administered at a dose of 20 mg/kg Q2W; and bevacizumab is administered at a dose of 5 mg/kg on days 1 and 15.

In some embodiments, the magrolimab and the bevacizumab are administered for first, second and third 28-day cycles, wherein:
a) for the first 28-day cycle, magrolimab is administered at a dose of 1 mg/kg on day 1 and at a dose of 15 mg/kg weekly (QW) beginning on day 8; and bevacizumab is administered at a dose of 5 mg/kg on days 1 and 15;
b) for the second 28-day cycle, magrolimab is administered at a dose of 15 mg/kg weekly (QW); and bevacizumab is administered at a dose of 5 mg/kg on days 1 and 15; and
c) for the third 28-day cycle, magrolimab is administered at a dose of 15 mg/kg Q2W; and bevacizumab is administered at a dose of 5 mg/kg on days 1 and 15.

In some embodiments, the agent that inhibits binding between CD47 and SIRPα; and the VEGFA/VEGFR inhibiting agent are administered in a combined synergistic amount. A “combined synergistic amount” as used herein refers to the sum of a first amount (e.g., an amount of an agent that inhibits binding between CD47 and SIRPα) and a second amount (e.g., an amount of a VEGFA/VEGFR inhibiting agent) that results in a synergistic effect (i.e., an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

Co-administration of an agent that inhibits binding between CD47 and SIRPα and a VEGFA/VEGFR inhibiting agent can allow for lower doses of one or both therapeutic agents. In embodiments, a synergistic amount may be about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the agent that inhibits binding between CD47 and SIRPα when used separately from the VEGFA/VEGFR inhibiting agent. In embodiments, a synergistic amount may be about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of a VEGFA/VEGFR inhibiting agent when used separately from the agent that inhibits binding between CD47 and SIRPα.

Dosage and frequency may vary depending on the half-life of the therapeutic agent in the patient. It will be understood by one of skill in the art that such guidelines will be adjusted for the molecular weight of the active agent, e.g., in the use of antibody fragments, in the use of antibody conjugates, in the use of SIRPα reagents, in the use of soluble CD47 peptides etc. The dosage may also be varied for localized administration, e.g., intranasal, inhalation, etc., or for systemic administration, e.g. intramuscular (i.m.), intraperitoneal (i.p.), intravenous (i.v.), subcutaneous (s.c.), intratumoral, intracranial, as appropriate. In some embodiments, the agent that inhibits binding between CD47 and SIRPα; and the VEGFA/VEGFR inhibiting agent are administered concurrently. In some embodiments, the agent that inhibits binding between CD47 and SIRPα; and the VEGFA/VEGFR inhibiting agent are administered sequentially. For example, the agent that inhibits binding between CD47 and SIRPα, described herein, may be administered within seconds, minutes, hours or days of the administration of the VEGFA/VEGFR inhibiting agent. In some embodiments, a unit dose of an agent that inhibits binding between CD47 and SIRPα is administered first, followed within seconds, minutes, hours or days by administration of a unit dose of a VEGFA/VEGFR inhibiting agent. Alternatively, a unit dose of a VEGFA/VEGFR inhibiting agent is administered first, followed by administration of a unit dose of an agent that inhibits binding between CD47 and SIRPα within seconds, minutes, hours or days. In other embodiments, a unit dose of an agent that inhibits binding between CD47 and SIRPα is administered first, followed, after a period of hours (e.g., 1-12 hours, 1-24 hours, 1-36 hours, 1-48 hours, 1-60 hours, 1-72 hours), by administration of a unit dose of a VEGFA/VEGFR inhibiting agent. In yet other embodiments, a unit dose of a VEGFA/VEGFR inhibiting agent is administered first, followed, after a period of hours (e.g., 1-12 hours, 1-24 hours, 1-36 hours, 1-48 hours, 1-60 hours, 1-72 hours), by administration of a unit dose of an agent that inhibits binding between CD47 and SIRPα.

6. Conditions Subject to Treatment

Provided are methods of treating, ameliorating, mitigating, or preventing or delaying the recurrence or metastasis of, a colorectal cancer (CRC) in a subject comprising administering: (a) an agent that inhibits binding between CD47 and SIRPα; and (b) a VEGFA/VEGFR inhibiting agent to the subject. In various embodiments, the CRC is an adenocarcinoma, a gastrointestinal carcinoid tumor, a primary colorectal lymphoma, a squamous cell carcinoma, a gastrointestinal stromal tumor (GIST), a leiomyosarcoma or a melanoma. In some embodiments, the CRC is a familial or hereditary colorectal cancer, such as a hereditary nonpolyposis colorectal cancer (HNPCC) or a familial adenomatous polyposis (FAP). Other types of colorectal cancers that can be treated by the herein described methods include, e.g., Turcot Syndrome, Peutz-Jeghers Syndrome (PJS), Familial Colorectal Cancer (FCC) and Juvenile Polyposis coli. In some embodiments, the subject is a human.

As used herein, “treatment” or “treating” is an approach for obtaining beneficial or desired results including clinical results. For example, beneficial or desired clinical results may include one or more of the following: (i) decreasing one more symptoms resulting from the disease; (ii) diminishing the extent of the disease, stabilizing the disease (e.g., preventing or delaying the worsening of the disease); (iii) preventing or delaying the spread (e.g., metastasis) of the disease; (iv) preventing or delaying the occurrence or recurrence of the disease, delay or slowing the progression of the disease; (v) ameliorating the disease state, providing a remission (whether partial or total) of the disease, decreasing the dose of one or more other medications required to treat the disease; (vi) delaying the progression of the disease, increasing the quality of life, and/or (vii) prolonging survival. The beneficial or desired clinical results may be observed in more patients or subjects who have received the methods or treatments described herein.

In various embodiments, the methods described herein are directed to treating, ameliorating, mitigating, reducing, preventing or delaying the recurrence or metastasis of, a colorectal cancer (CRC), e.g., metastatic CRC, e.g., inoperable CRC, e.g., CRC that has progressed following at least one prior therapy. In some embodiments, the cancer has progressed following at least one prior anti-cancer therapy. In some embodiments, the cancer has progressed following at least one prior anti-cancer therapy selected from an anti-VEGFA therapy (e.g., bevacizumab, aflibercept), a chemotherapy regimen including platinum coordination complex (e.g., FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), XELIRI (capecitabine, irinotecan), XELOXIRI (capecitabine, oxaliplatin, irinotecan)), a platinum coordination complex therapy (e.g., cisplatin, oxiloplatinim, and carboplatin), an immune checkpoint inhibitor therapy (e.g., anti-PD1 antibody therapy or an anti-PD-L1 antibody therapy). In some embodiments, the CRC has progressed following therapy with one or more of the following therapeutic agents in the absence of magrolimab: bevacizumab, capecitabine, cetuximab, fluorouracil, irinotecan, leucovorin, oxaliplatin, panitumumab, ziv-aflibercept, and any combinations thereof. In some embodiments therapeutic agents used to treat CRC include bevacizumab (AVASTIN®), leucovorin, 5-FU, oxaliplatin (FOLFOX), pembrolizumab (KEYTRUDA®), FOLFIRI, regorafenib (STIVARGA®), aflibercept (ZALTRAP®), cetuximab (ERBITUX®), Lonsurf (ORCANTAS®), XELOX, FOLFOXIRI, or a combination thereof. In some embodiments therapeutic agents used to treat CRC include bevacizumab+leucovorin+5-FU+oxaliplatin (FOLFOX), bevacizumab+FOLFIRI, bevacizumab+FOLFOX, aflibercept+FOLFIRI, cetuximab+FOLFIRI, bevacizumab+XELOX, and bevacizumab+FOLFOXIRI. In some embodiments therapeutic agents used to treat CRC include binimetinib+encorafenib+cetuximab, trametinib+dabrafenib+panitumumab, trastuzumab+pertuzumab, napabucasin+FOLFIRI+bevacizumab, nivolumab+ipilimumab.

In various embodiments, the CRC is classified as stage I, i.e., according to the TNM classification system. In various embodiments, the CRC is classified as stage II (e.g., IIA, IIB or IIC), i.e., according to the TNM classification system. In various embodiments, the CRC is classified as stage III (e.g., IIIA, IIIB or IIIC), i.e., according to the TNM classification system. In various embodiments, the CRC is classified as stage IV (e.g., IVA, IVB or IVC), i.e., according to the TNM classification system. See, Delattre, et al., Cancer Treat Rev. (2022) February; 103:102325.

In some embodiments, the subject is treatment naïve, i.e., combined administration of an agent that inhibits binding between CD47 and SIRPα (e.g., magrolimab) and a VEGFA/VEGFR inhibiting agent (e.g., bevacizumab) is a first line cancer therapy.

“Prevention” or “preventing” means any treatment (i.e., medication, drug, therapeutic) of a disease or condition (i.e., cancer) that causes the clinical symptoms of the disease or condition not to develop. Compounds may, in some embodiments, be administered to a subject (including a human) who is at risk or has a family history of the disease or condition.

“Delaying” the development of a cancer means to defer, hinder, slow, retard, stabilize, and/or postpone development of the disease. The delay can be of varying lengths of time, depending on the history of the disease and/or subject being treated. As is evident to one of skill in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Disease development can be detectable using standard methods, such as routine physical exams, blood draw, mammography, imaging, or biopsy. Development may also refer to disease progression that may be initially undetectable and includes occurrence, recurrence, and onset.

The term “ameliorating” refers to any therapeutically beneficial result in the treatment of a disease state, e.g., a cancer disease state, including prophylaxis, lessening in the severity or progression, remission, or cure thereof.

7. Kits

Also described herein are kits comprising one or more unitary doses of the active agents, e.g., an agent that inhibits binding between CD47 and SIRPα (e.g., magrolimab) and a VEGFA/VEGFR inhibiting agent (e.g., bevacizumab), and formulations thereof, as described herein, and instructions for use. In various embodiments, the agent that inhibits binding between CD47 and SIRPα and the VEGFA/VEGFR inhibiting agent can be in the same or different containers. The kit can further contain a least one additional reagent, e.g. agents for a chemotherapy regimen (e.g., FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), XELIRI (capecitabine, irinotecan), XELOXIRI (capecitabine, oxaliplatin, irinotecan)), an immune checkpoint inhibitor. Kits typically include a label indicating the intended use of the contents of the kit. The term label includes any writing, or recorded material supplied on or with the kit, or which otherwise accompanies the kit.

In some embodiments, one or both of the agent that inhibits binding between CD47 and SIRPα (e.g., magrolimab) and the VEGFA/VEGFR inhibiting agent (e.g., bevacizumab) are provided in a dosage form (e.g., a therapeutically effective dosage form). In some embodiments, one or both of the agent that inhibits binding between CD47 and SIRPα (e.g., magrolimab) and the VEGFA/VEGFR inhibiting agent (e.g., bevacizumab) are provided in two or more different dosage forms (e.g., two or more different therapeutically effective dosage forms). In the context of a kit, one or both of the agent that inhibits binding between CD47 and SIRPα (e.g., magrolimab) and the VEGFA/VEGFR inhibiting agent (e.g., bevacizumab) can be provided in liquid or sold form in any convenient packaging (e.g., stick pack, dose pack, etc.).

The agent that inhibits binding between CD47 and SIRPα (e.g., magrolimab) and a VEGFA/VEGFR inhibiting agent (e.g., bevacizumab) can be provided in the same or separate containers, as appropriate. In various embodiments, the agent that inhibits binding between CD47 and SIRPα (e.g., magrolimab) and the VEGFA/VEGFR inhibiting agent (e.g., bevacizumab) are provided in separate containers. Compositions comprising one or both of an agent that inhibits binding between CD47 and SIRPα (e.g., magrolimab) and a VEGFA/VEGFR inhibiting agent (e.g., bevacizumab) are provided in one or more containers, the containers having a label. Suitable containers include, for example, bottles, vials, ampoules, syringes (including pre-loaded syringes), and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in one composition is the agent that inhibits binding between CD47 and SIRPα (e.g., the anti-CD47 antibody, e.g., magrolimab). The active agent in a second composition is a VEGFA/VEGFR inhibiting agent (e.g., bevacizumab). The label on, or associated with, the container indicates that the composition is used for treating the condition of choice. The article of manufacture may further comprise one or more containers comprising a pharmaceutically-acceptable buffer, e.g., for use as diluent. Illustrative buffers include without limitation phosphate-buffered saline, Ringer's solution and/or dextrose solution. The kit may further include other materials desirable from a commercial and user standpoint, including other buffers, diluents, filters, needles, syringes, and package inserts with instructions for use.

In various embodiments, the subject kits include a primer agent (e.g., an erythropoiesis-stimulating agent (ESA)) and an anti-CD47 agent. In some embodiments, a kit comprises two or more primer agents. In some embodiments, a kit comprises two or more anti-CD47 agents. In some embodiments, a primer agent is provided in a dosage form (e.g., a priming dosage form). In some embodiments, a primer agent is provided in two or more different dosage forms (e.g., two or more different priming dosage forms).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### EXAMPLES

The following examples are offered to illustrate, but not to limit the claimed invention.

### Example 1

### A Phase 2, Randomized, Open-Label Study Evaluating the Safety and Efficacy of Magrolimab in Combination with Bevacizumab and FOLFIRI Versus Bevacizumab and FOLFIRI in Previously Treated Advanced Inoperable Metastatic Colorectal Cancer (mCRC)

### Objectives and Endpoints

Table 1 presents the study objectives and end points.

TABLE 1
Study Objectives and End Points

| Primary Objectives | Primary Endpoints |
|---|---|
| Safety Run Cohort: | Safety Run Cohort: |
| To evaluate the safety, tolerability, | Incidence of dose-limiting toxicities |
| and recommended Phase 2 dose | (DLTs), and adverse events (AEs) |
| (RP2D) of magrolimab in | and laboratory abnormalities |
| combination with bevacizumab and | according to National Cancer |
| FOLFIRI (5-fluorouracil [5-FU], | Institute (NCI) Common |
| irinotecan, and leucovorin [LV]) in | Terminology Criteria for Adverse |
| previously treated patients with | Events (CTCAE), Version 5.0 |
| advanced inoperable metastatic | Randomized Cohort: |
| colorectal cancer (mCRC) | PFS, defined as the time from the |
| Randomized Cohort: | date of randomization until the |
| To evaluate the efficacy of | earliest date of documented disease |
| magrolimab in combination with | progression as determined by |
| bevacizumab and FOLFIRI in mCRC | investigator assessment using |
| as determined by progression-free | response evaluation criteria in solid |
| survival (PFS) by investigator | tumors Version 1.1 (RECIST V1.1), |
| assessment | or death from any cause, whichever |
| | occurs first |
| Secondary Objectives | Secondary Endpoints |
| Randomized Cohort: | Randomized Cohort: |
| To evaluate objective response rate | Confirmed ORR, defined as the |
| (ORR) and PFS by independent | proportion of patients with complete |
| central review, and ORR by | response (CR) or partial response (PR) |
| investigator assessment | on 2 consecutive assessments, at least 28 |
| To evaluate additional measures of | days apart, as determined by investigator |
| efficacy of magrolimab in | assessment and independent central |
| combination with bevacizumab and | review using RECIST V1.1 |
| FOLFIRI, including duration of | PFS, defined as the time from the date of |
| response (DOR) and overall survival | randomization until the earliest date of |
| (OS) | documented disease progression, as |
| To evaluate patient-reported | determined by independent central review |
| outcomes (PRO)/quality-of-life | using RECIST V1.1, or death from any |
| measures for the Randomized Cohort | cause, whichever occurs first |
| in mCRC with magrolimab in | DOR, defined as time from first |
| combination with bevacizumab and | documentation of CR or PR to the |
| FOLFIRI | earliest date of documented disease |
| Safety Run and Randomized Cohorts: | progression as determined by |
| To evaluate the pharmacokinetics | investigator assessment, per RECIST |
| (PK) and immunogenicity of | V1.1, or death from any cause, |
| magrolimab in combination with | whichever occurs first |
| bevacizumab and FOLFIRI | DOR, defined as time from first |
| | documentation of CR or PR to the |
| | earliest date of documented disease |
| | progression as assessed by independent |
| | central review, per RECIST V1.1, or |
| | death from any cause, whichever occurs |
| | first |
| | OS, defined as time from date of |
| | randomization to death from any cause |
| | PRO assessments (European |
| | Organisation for Research and Treatment |
| | of Cancer Quality of Life Questionnaire |
| | - Core Questionnaire [EORTC-QLQ- |
| | C30], the 5-level EuroQol 5 dimensions |
| | questionnaire [EQ-5D-5L]) scores, and |
| | Functional Assessment of Cancer |
| | Therapy [FACT] Colorectal Symptom |
| | Index [FCSI]) |
| | Safety Run and Randomized Cohorts: |
| | Magrolimab concentration versus time |
| | and antidrug antibodies (ADA) to |
| | magrolimab |
| Exploratory Objectives | Exploratory Endpoints |
| To evaluate the pharmacodynamic (PD), | Change in the PD, MOA, and/or |
| mechanism of action (MOA), and/or | therapeutic response biomarkers in blood |
| therapeutic response biomarkers in blood | and tumor biopsy samples |
| and tumor biopsy samples | Correlation of clinical response with |
| To explore biomarkers that may predict | biomarkers at baseline and/or on |
| response to therapy | treatment |

### Study Design

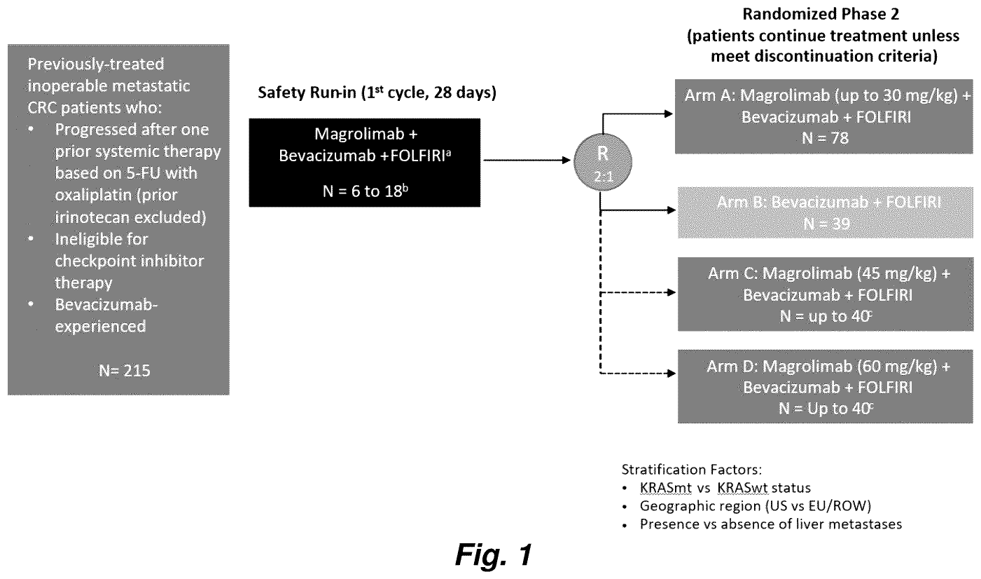

A schematic diagram of the study is provided in FIG. 1.

This is a Phase 2, randomized, open-label, multicenter study to evaluate magrolimab in combination with bevacizumab and FOLFIRI in previously treated patients with advanced inoperable mCRC who have progressed after one prior systemic therapy and who do not harbor BRAF V600E mutations or high microsatellite instability (MSI-H).

This study will involve the following 2 cohorts:

Safety Run Cohort: magrolimab in combination with bevacizumab and FOLFIRI in previously treated patients with advanced inoperable mCRC.

After completion of the Safety Run Cohort, the Randomized Cohort will be open to enrollment.

Randomized Cohort: magrolimab in combination with bevacizumab and FOLFIRI (Experimental Arm A) versus bevacizumab and FOLFIRI (Control Arm B) in previously treated patients with advanced inoperable mCRC.

### Safety Run Cohort:

Initially, 6 patients will be enrolled in the Safety Run Cohort at a starting dose level. A DLT-assessment period of 28 days will occur.

Although no dose-dependent toxicities have been observed with magrolimab, in order to preserve the efficacious doses of the combination partner drugs, dose de-escalation will take place for magrolimab as follows:
If 2 or less of 6 DLT-evaluable patients experience a DLT during the first 28 days, enrollment into the Randomized Cohort will begin at this dose level as the RP2D.
If more than 2 patients experience at least one DLT during the first 28 days, enrollment at the current dose level will immediately stop and dose de-escalation will occur. Up to 6 other patients will then be enrolled and evaluated at a lower dose level in the same manner.
Once the RP2D is determined, the sponsor will open the Randomized Cohort.

Approximately 6 patients will be enrolled in the Safety Run Cohort. Additional patients may be added to the Safety Run Cohort or enrolled in a dose-de-escalation cohort.

Dose-Limiting Toxicity (DLT) Assessment Period for the Safety Run Cohort: The DLT assessment period will be the first 28 days and applies to the Safety Run Cohort. Patients are considered evaluable for assessment of a DLT if either of the following criteria is met in the DLT-assessment period:
The patient experienced a DLT at any time after initiation of the first infusion of magrolimab.
The patient did not experience a DLT and completes at least 3 infusions of magrolimab and at least 2 doses of bevacizumab and FOLFIRI in the Safety Run Cohort.

If a patient experiences a DLT during the DLT-assessment period, the patient will discontinue treatment. Patients who are not evaluable for DLT assessment in the Safety Run Cohort will be replaced.

Randomized Cohort: Once the Safety Run Cohort is completed and the RP2D for magrolimab in combination with bevacizumab and FOLFIRI is determined, the sponsor will open the Randomized Cohort. In this open-label, randomized, 2 arm study, patients with mCRC will be randomized in a 2:1 ratio to receive either magrolimab in combination with bevacizumab and FOLFIRI (Experimental Group A) or bevacizumab and FOLFIRI (Control Group B). The primary efficacy assessment will be investigator assessed PFS, with the primary analysis to occur after 85 PFS events. Stratification factors for randomization include the following: (1) Kirsten rat sarcoma (KRAS) mutation versus wild-type status, (2) geographic region (United States [US] versus European Union [EU]/rest of world [ROW]), (3) presence versus absence of liver metastases.

### Duration of Treatment

All patients will continue study treatment unless they meet study treatment discontinuation criteria. Survival follow-up will be conducted via a phone call every 2 months until death or end of study. Duration of survival follow-up will be limited to 3 years.

### Target Population

Previously treated patients with advanced inoperable mCRC who have progressed after 1 prior systemic therapy and who do not harbor BRAF V600E mutations or MSI-H. Patients with mismatch repair deficiency will be excluded. Patients can be included regardless of KRAS mutation status, however. Magrolimab mechanism of action is believed to support development in both KRAS wild-type and mutant settings. Data from the 5F9004 study (i.e., NCT02953782) did not identify a subpopulation that would specifically benefit from magrolimab. KRAS-mutated CRC is present in 45% of the patients, portends a poor prognosis, and is challenging to target.

### Diagnosis and Main Eligibility Criteria:

### Inclusion Criteria

All patients must meet all of the following inclusion criteria to be eligible for participation in this study:
1) Patient has provided informed consent.
2) Patient is willing and able to comply with clinic visits and procedures outlined in the study protocol.
3) Male or female, at least 18 years of age.
4) Previously treated patients with inoperable mCRC who have progressed on or after 1 prior systemic therapy and who are ineligible for checkpoint inhibitor therapy (eligible patients for checkpoint inhibitor therapy are defined as MSI-H or mismatch repair deficient [dMMR] and are excluded). Note: Maintenance therapies are not counted as separate lines of therapy.
5) Chemotherapy and bevacizumab (if applicable) free interval of at least 3 weeks.
6) Histologically or cytologically confirmed adenocarcinoma originating in the colon or rectum (excluding appendiceal and anal canal cancers), who have progressed on or after 1 prior systemic therapy in the setting where curative resection is not indicated. This therapy must have included chemotherapy based on 5-fluorouracil (5-FU) with oxaliplatin and either bevacizumab, or for patients with RAS wild-type and left-sided tumors, bevacizumab or cetuximab or panitumumab.
7) Measurable disease (at least 1 measurable metastatic lesion by RECIST V1.1 criteria, with lesion not located in a previous field of radiation). Previously irradiated lesions can be considered as measurable disease only if disease progression has been unequivocally documented at that site since radiation.
8) Patients must have an ECOG performance status of 0 or 1.
9) Life expectancy of at least 12 weeks.
10) Laboratory measurements, blood counts:
   a. Hemoglobin (Hb) must be ≥9 g/dL prior to initial dose of study treatment.
      NOTE: Transfusions are allowed to meet Hb eligibility.
   b. Absolute neutrophil count at least 1.5×10⁹/L
   c. Platelets at least 100×10⁹/L
11) Adequate liver function, as demonstrated by:
   a. AST less than or equal to 2.5×ULN or less than or equal to 5×ULN in patients with liver metastases
   b. ALT less than or equal to 2.5×ULN or less than or equal to 5×ULN in patients with liver metastases
   c. Bilirubin less than or equal to 1.5×ULN, or less than or equal to 3.0×ULN and primarily unconjugated if patient has a documented history of Gilbert's syndrome or genetic equivalent.
12) Patients must have adequate renal function as demonstrated by a creatinine clearance of at least 30 mL/min; calculated by the Cockcroft Gault formula.
13) Pretreatment blood cross-match completed.
14) Male and female patients of childbearing potential who engage in heterosexual intercourse must agree to use protocol specified method(s) of contraception

### Exclusion Criteria

Patients who meet any of the following exclusion criteria are not to be enrolled in this study:
1) Prior anticancer therapy including chemotherapy, hormonal therapy, or investigational agents within 3 weeks or within at least 4 half-lives prior to magrolimab dosing (up to a maximum of 4 weeks), whichever is shorter.
2) Known BRAF V600E or MSI-H mutations or dMMR.
3) Persistent Grade 2 or more gastrointestinal bleeding.
4) Patients who received a dose-escalation scheme (e.g., LV/5-FU with bevacizumab followed by FOLFOX or FOLFIRI with bevacizumab) and/or who are not tolerant to bevacizumab.
5) Patients with prior irinotecan therapy.
6) Patients without prior bevacizumab therapy for colorectal cancer.
7) Patient with known allergy or hypersensitivity to monoclonal antibodies (bevacizumab, magrolimab), or to Chinese hamster ovarian cell products or any other humanized or recombined antibodies or any other chemotherapies under study, and their excipients.
8) Clinically significant coronary artery disease or myocardial infarction within 6 months prior to inclusion.
9) Peripheral neuropathy of more than Grade 1 (CTCAE Version 5.0).
10) Known dihydropyrimidine dehydrogenase deficiency.
11) Acute intestinal obstruction or subobstruction, history of inflammatory intestinal disease or extended resection of the small intestine. Presence of a colonic prosthesis.
12) Unhealed wound, active gastric or duodenal ulcer, or bone fracture.
13) History of abdominal fistulas, trachea-oesophageal fistulas, any other Grade 4 gastrointestinal perforations, nongastrointestinal fistulas, or intra-abdominal abscesses 6 months prior to screening.
14) Uncontrolled arterial hypertension (systolic pressure>150 mm Hg and/or diastolic pressure>100 mm Hg) with and without antihypertensive medication.
15) History of hypertensive crisis or hypertensive encephalopathy.
16) Thromboembolic event in the 6 months before inclusion (e.g., transitory ischemic stroke, stroke, subarachnoid hemorrhage) except peripheral deep vein thrombosis treated with anticoagulants.
17) Positive serum pregnancy test.
18) Breastfeeding female.
19) Active central nervous system (CNS) disease. Patients with asymptomatic and stable, treated CNS lesions (radiation and/or surgery and/or other CNS-directed therapy who have not received corticosteroids for at least 4 weeks) are allowed.
20) RBC transfusion dependence, defined as requiring more than 2 units of packed RBC transfusions during the 4-week period prior to screening. RBC transfusions are permitted during the screening period and prior to enrollment to meet the Hb inclusion criteria.
21) History of hemolytic anemia, autoimmune thrombocytopenia, or Evans syndrome in the last 3 months.
22) Known hypersensitivity to any of the study drugs, the metabolites, or formulation excipient.
23) Prior treatment with CD47 or signal regulatory protein alpha targeting agents.
24) Current participation in another interventional clinical study.
25) Known inherited or acquired bleeding disorders.
26) Significant disease or medical conditions, as assessed by the investigator and sponsor, that would substantially increase the risk benefit ratio of participating in the study. This includes, but is not limited to, acute myocardial infarction within the last 6 months, unstable angina, uncontrolled diabetes mellitus, significant active infections, and congestive heart failure New York Heart Association Class III IV.
27) Second malignancy, except treated basal cell or localized squamous skin carcinomas, localized prostate cancer, or other malignancies for which patients are not on active anticancer therapies and who are in complete remission for over 2 years.
28) Known active or chronic hepatitis B or C infection or HIV infection in medical history.
29) Uncontrolled pleural effusion.
30) Uncontrolled hypercalcemia (ionized calcium>1.5 mmol/L) or symptomatic hypercalcemia requiring continued use of bisphosphonate therapy.
31) Uncontrolled tumor-related pain.
32) Severe/serious systemic infection within 4 weeks of randomization.

NOTE: Localized non-CNS radiotherapy, previous hormonal therapy with luteinizing hormone releasing hormone agonists for breast cancer, and treatment with bisphosphonates and receptor activator of nuclear factor kappa B ligand (RANKL) inhibitors are not criteria for exclusion. There is no required minimum washout period for these therapies. Patients should be recovered from the effects of radiation.

### FOLFIRI as Chemotherapy Backbone

The decision to incorporate FOLFIRI is based on biology/MOA, the treatment landscape, as well as the standard of care (SOC) in advanced colorectal cancer. FOLFIRI is the preferred chemotherapy backbone in second-line mCRC in the US and is used interchangeably with FOLFOX in the European Union (EU). Fluorouracil, a fluoropyrimidine, is reported to relieve immunosuppression by reducing myeloid-derived immunosuppressive cells such as myeloid-derived suppressor cells. Irinotecan has also been shown to upregulate the expression of immunogenic cell death markers and promote phagocytosis of tumor cells by phagocytes (Pozzi, et al., Nat Med (2016) 22 (6):624-31).

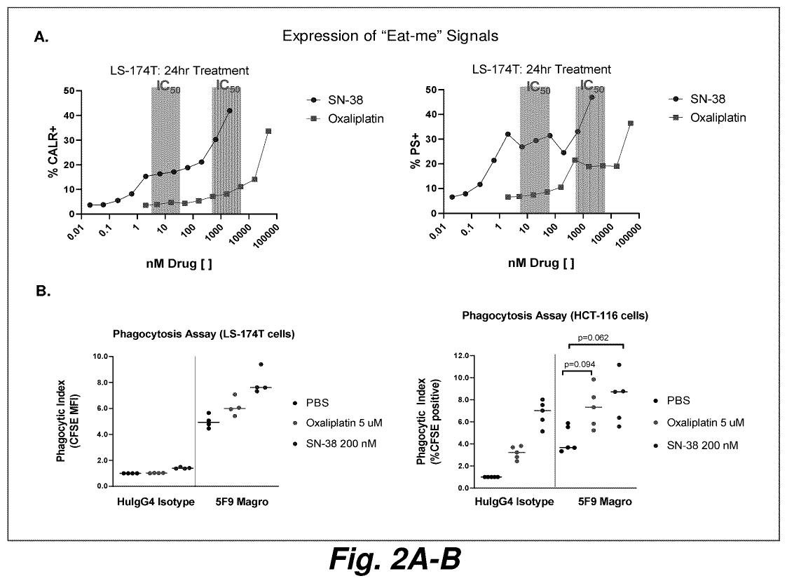

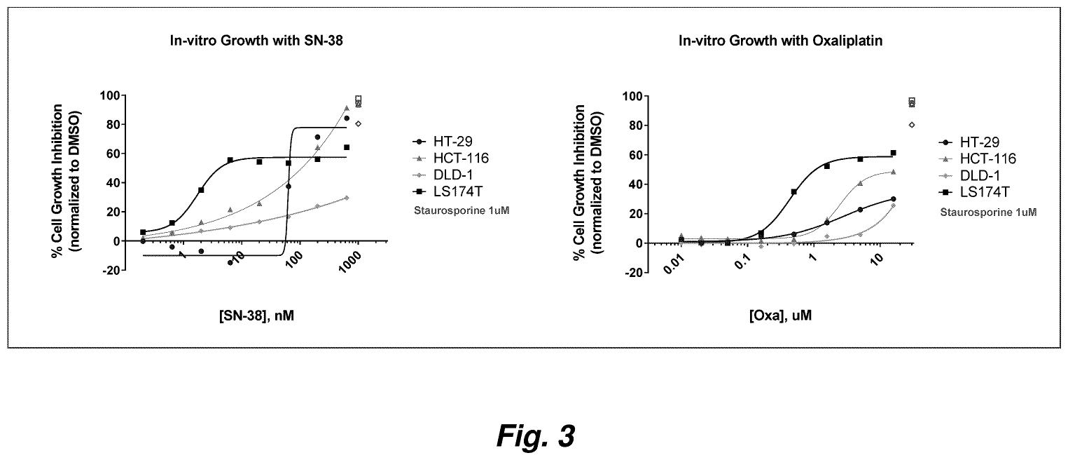

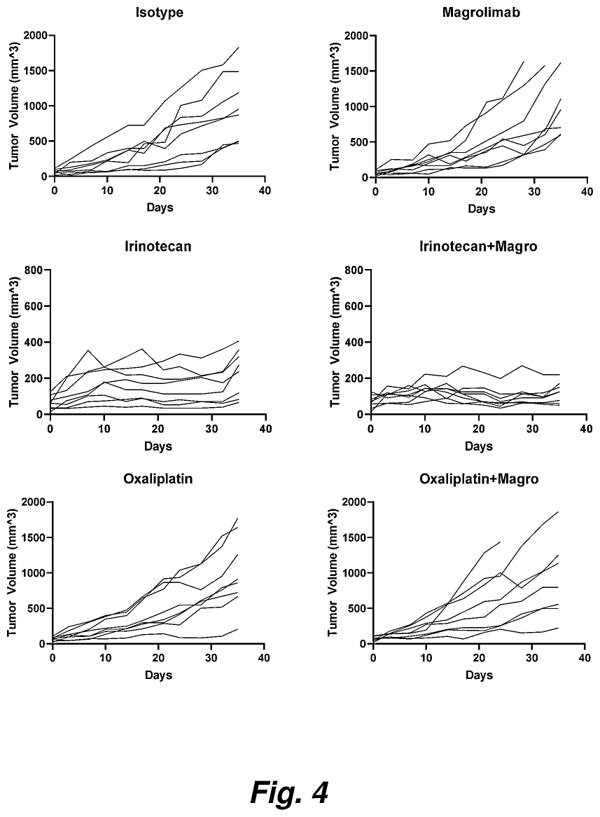

Data presented herein are consistent with the conclusion that irinotecan was more effective than oxaliplatin at increasing the cell surface expression of prophagocytic receptors and promoting the phagocytosis of colorectal cancer cells, irrespective of their mutational profile (FIG. 2). The CRC cells treated with irinotecan were also more likely to undergo growth inhibition (FIG. 3). In immunocompromised mice bearing subcutaneous HT-29 tumors, combinations of magrolimab and irinotecan provided superior tumor growth control compared with magrolimab and oxaliplatin (FIG. 4). These findings support the use of FOLFIRI as a chemotherapeutic backbone to magrolimab.

### Dosing and Administration

The magrolimab dosing regimen for the safety run-in and randomized cohorts is presented in Table 2 and Table 3.

TABLE 2
Safety Run Cohort Dosing and Dose-De-escalation Regimen

| | Dose Schedule (Day per 28-Day Cycle) | | |
|---|---|---|---|
| Drug/Dose/Route | Cycle 1 | Cycle 2 | Cycle 3+ |
| Bevacizumab | Days 1, 15 | Days 1, 15 | Days 1, 15 |
| 5 mg/kg IV every 2 weeks^{a} | | | |
| FOLFIRI IV^{b} | Days 1, 2, 15, 16 | Days 1, 2, 15, 16 | Days 1, 2, 15, 16 |
| Irinotecan 180 mg/m² over 30-90 minutes on first day of dose | | | |
| administration | | | |
| Leucovorin 400 mg/m² over 2 hours on first day of dose | | | |
| administration | | | |
| Fluorouracil 400 mg/m² bolus on first day of dose administration, | | | |
| followed by 2400 mg/m² over 46 hours, continuous infusion | | | |

Magrolimab Administration

| | |
|---|---|
| Magrolimab | Day 1 |
| 1 mg/kg IV (3 hours ± 30 min) | |
| Magrolimab starting dose level | QW beginning at Day 8 visit and the next 6 doses |
| 30 mg/kg IV (2 hours ± 30 min) | (e.g., Cycle 1 [Week 1, Week 2, Week 3], Cycle 2 [Week 4, Week 5, Week 6, |
| | Week 71) |
| Magrolimab | Q2W beginning 1 week after the last QW 30 mg/kg dose |
| 30 mg/kg IV (2 hours ± 30 min) | (e.g., starting Cycle 3 [Week 8] onward) |
| Magrolimab de-escalation Level - 1 | QW beginning at Day 8 visit and the next 6 doses |
| 20 mg/kg IV (2 hours ± 30 min) | (e.g., Cycle 1 [Week 1, Week 2, Week 3], Cycle 2 [Week 4, Week 5, Week 6, |
| | Week 7]) |
| | Q2W beginning 1 week after the last weekly 20 mg/kg dose |
| | (e.g., starting Cycle 3 [Week 8] onward) |
| Magrolimab de-escalation Level - 2 | QW beginning at Day 8 and the next 6 doses |
| 15 mg/kg IV (2 hours ± 30 min) | (e.g., Cycle 1 [Week 1, Week 2, Week], Cycle 2 [Week 4, Week 5, Week 6, |
| | Week 71) |
| | Q2W beginning 1 week after the last weekly 15 mg/kg dose (e.g., starting Cycle |
| | 3 [Week 8] onward) |

FOLFIRI = 5-fluorouracil, irinotecan, and leucovorin;
IV = intravenous;
QW = every week;
Q2W = every 2 weeks
^{a}Bevacizumab should be administered per standard of care and/or institutional guidelines. Recommendations include administering the first dose within 60-90 minutes. If well tolerated, subsequent infusions may be administered within 10 to 30 minutes.
^{b}FOLFIRI should be administered per standard of care and/or institutional guidelines and described in detail in the table.

TABLE 3
Randomized Cohort Dosing Regimen

| | Dose Schedule (Day per 28-Day Cycle) | | |
|---|---|---|---|
| Drug/Dose/Route | Cycle 1 | Cycle 2 | Cycle 3+ |
| Bevacizumab | Days 1, 15 | Days 1, 15 | Days 1, 15 |
| 5 mg/kg IV every 2 weeks^{a} | | | |
| FOLFIRI IV^{b} | Days 1, 2, 15, 16 | Days 1, 2, 15, 16 | Days 1, 2, 15, 16 |
| Irinotecan 180 mg/m² over 30-90 minutes on first day of dose administration | | | |
| Leucovorin 400 mg/m² over 2 hours on first day of dose administration | | | |
| Fluorouracil 400 mg/m² bolus on first day of dose administration, followed by 2400 | | | |
| mg/m² over 46 hours, continuous infusion | | | |

Magrolimab Administration

| | |
|---|---|
| Magrolimab | Day 1 |
| 1 mg/kg IV (3 hours ± 30 min) | |
| Magrolimab | QW beginning at Day 8 visit and the next 6 doses |
| RP2D IV (2 hours ± 30 min) | (eg, Cycle 1 [Week 1, Week 2, Week 3], Cycle 2 [Week 4, |
| | Week 5, Week 6, Week 7]) |
| Magrolimab | Q2W beginning 1 week after the last weekly RP2D |
| RP2D IV (2 hours ± 30 min) | (e.g., starting Cycle 3 [Week 8] onward) |

FOLFIRI = 5-fluorouracil, irinotecan, and leucovorin;
IV = intravenous;
QW = every week;
Q2W = every 2 weeks;
RP2D = recommended Phase 2 dose
^{a}Bevacizumab should be administered per standard of care and/or institutional guidelines. Recommendations include administering the first dose within 60-90 minutes. If well tolerated, subsequent infusions may be administered within 10 to 30 minutes.
^{b}FOLFIRI should be administered per standard of care and/or institutional guidelines and described in detail in the table.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating previously treated advanced inoperable metastatic colorectal cancer in a subject comprising co-administering to the subject an effective amount of:
a) an agent that inhibits binding between CD47 and SIRPα; and
b) an agent that inhibits binding between vascular endothelial growth factor A (VEGFA) and one or more VEGFA cognate receptors.

2-45. (canceled)

46. A method of treating, mitigating, reducing, preventing, or delaying the recurrence or metastasis of, colorectal cancer in a subject comprising co-administering to the subject an effective amount of magrolimab and bevancizumab.

47. The method of claim 46, wherein the cancer is (i) unresectable, locally advanced or (ii) metastatic.

48. The method of claim 46, wherein the cancer is unresectable, locally advanced and the subject is treatment naïve.

49. A method of treating previously treated advanced inoperable metastatic colorectal cancer in a subject comprising co-administering to the subject an effective amount of:
a) magrolimab; and
b) bevancizumab.

50. The method of claim 46, further comprising co-administering a chemotherapy regimen.

51. The method of claim 46, comprising co-administering a FOLFOX regimen, a FOLFIRI regimen, a XELIRI (a.k.a., CAPIRI) regimen, a FOLFOXIRI regimen, a XELOXIRI regimen or a FOLFIRINOX regimen.

52. The method of claim 46, comprising co-administering a FOLFIRI regimen or a XELIRI regimen.

53. The method of claim 46, wherein the cancer has cell surface expression of CD47.

54. The method of claim 46, wherein the cancer does not comprise a BRAF V600E mutation.

55. The method of claim 46, wherein the cancer does not comprise high microsatellite instability.

56. The method of claim 46, wherein the cancer has one or more KRAS mutations and the subject has not responded to anti-EGFR antibody therapy.

57. The method of claim 46, wherein the cancer is adenocarcinoma originating in the colon or rectum.

58. The method of claim 46, wherein the cancer has progressed after one or more prior systemic therapies.

59. The method of claim 58, wherein the one or more prior therapies comprise administration of one or more agents selected from the group consisting of 5-fluorouracil (5-FU), oxaliplatin, bevacizumab, cetuximab and panitumumab.

60. The method of claim 46, wherein the treatment results in a reduction in overall tumor burden of at least 15%, at least 20%, at least 30%, or at least 40%, as determined using linear dimensional methods (e.g. RECIST v1.1).

61. The method of claim 47, comprising reducing in size or eliminating the metastases.

62. The method of claim 46, further comprising administering one or more therapeutic antibodies.

63. The method of claim 46, further comprising co-administering an antibody that binds to epidermal growth factor receptor (EGFR).

64. The method of claim 63, wherein the antibody that binds to EGFR is selected from cetuximab and panitumumab.

65. The method of claim 46, further comprising co-administering one or more blockers or inhibitors of one or more T-cell stimulatory immune checkpoint proteins or receptors.

66. The method of claim 65, wherein the one or more immune checkpoint inhibitors comprises a proteinaceous (e.g., antibody) inhibitor of PD-L1 (CD274), PD-1 (PDCD1) or CTLA4.

67. The method of claim 65, wherein the one or more immune checkpoint proteins or receptors are selected from: CD274 (CD274, PDL1, PD-L1) and programmed cell death 1 (PDCD1, PD1, PD-1).

68-72. (canceled)

73. The method of claim 46, wherein the magrolimab and the bevacizumab are administered in a combined synergistic amount.

74. The method of claim 46, wherein administration of the magrolimab and the bevacizumab provides a synergistic effect.

75. The method of claim 74, wherein the synergistic effect is increased cancer cell death and/or decreased cancer cell growth when comparing the effect of the combination versus either the magrolimab or the bevacizumab alone.

76. The method of claim 74, wherein the synergistic effect is increased phagocytosis of cancer cells by macrophages when comparing the effect of the combination versus either the magrolimab or the bevacizumab alone.

77. The method of claim 74, wherein the synergistic effect is increased or enhanced tumor burden reduction when comparing the effect of the combination versus either the magrolimab or the bevacizumab alone.

78. The method of claim 46, wherein the magrolimab is first administered at a priming dose of less than 10 mg/kg and then administered at one or more therapeutic doses of at least 15 mg/kg, e.g., at least 30 mg/kg, 45 mg/kg, 60 mg/kg.

79. The method of claim 46, wherein the magrolimab is first administered at a priming dose of less than 5 mg/kg and then administered at one or more therapeutic doses of at least 30 mg/kg, e.g., 45 mg/kg, 60 mg/kg.

80. The method of claim 46, wherein the magrolimab is first administered at a priming dose of 1 mg/kg, then administered at one or more therapeutic doses of 30 mg/kg, followed by administration of one or more therapeutic doses of 60 mg/kg.

81. The method of claim 46, wherein the magrolimab is first administered at a priming dose of 1 mg/kg, then administered at one or more therapeutic doses of 20 mg/kg, followed by administration of one or more therapeutic doses of 45 mg/kg.

82. The method of claim 46, wherein the magrolimab is first administered at a priming dose of 1 mg/kg, then administered at one or more therapeutic doses of 15 mg/kg, followed by administration of one or more therapeutic doses of 30 mg/kg.

83. The method of claim 46, wherein the magrolimab is administered intravenously, subcutaneously or intratumorally.

84. The method of claim 46, wherein the bevacizumab is administered at one or more doses in the range of 5 mg/kg to 15 mg/kg, e.g., 5 mg/kg to 7.5 mg/kg or 7.5 mg/kg to 15 mg/kg.

85. The method of claim 46, wherein the bevacizumab is administered at one or more doses of 5 mg/kg.

86. The method of claim 46, wherein the bevacizumab is administered intravenously, subcutaneously or intratumorally.

87. The method of claim 46, wherein the magrolimab and the bevacizumab are administered for first, second and third 28-day cycles, wherein:
a) for the first 28-day cycle, magrolimab is administered at a dose of 1 mg/kg on day 1 and at a dose of 30 mg/kg weekly (QW) beginning on day 8; and bevacizumab is administered at a dose of 5 mg/kg on days 1 and 15;
b) for the second 28-day cycle, magrolimab is administered at a dose of 30 mg/kg weekly (QW); and bevacizumab is administered at a dose of 5 mg/kg on days 1 and 15; and
c) for the third 28-day cycle, magrolimab is administered at a dose of 30 mg/kg Q2W; and bevacizumab is administered at a dose of 5 mg/kg on days 1 and 15.

88. The method of claim 46, wherein the magrolimab and the bevacizumab are administered for first, second and third 28-day cycles, wherein:
a) for the first 28-day cycle, magrolimab is administered at a dose of 1 mg/kg on day 1 and at a dose of 20 mg/kg weekly (QW) beginning on day 8; and bevacizumab is administered at a dose of 5 mg/kg on days 1 and 15;
b) for the second 28-day cycle, magrolimab is administered at a dose of 20 mg/kg weekly (QW); and bevacizumab is administered at a dose of 5 mg/kg on days 1 and 15; and
c) for the third 28-day cycle, magrolimab is administered at a dose of 20 mg/kg Q2W; and bevacizumab is administered at a dose of 5 mg/kg on days 1 and 15.

89. The method of claim 46, wherein the magrolimab and the bevacizumab are administered for first, second and third 28-day cycles, wherein:
a) for the first 28-day cycle, magrolimab is administered at a dose of 1 mg/kg on day 1 and at a dose of 15 mg/kg weekly (QW) beginning on day 8; and bevacizumab is administered at a dose of 5 mg/kg on days 1 and 15;
b) for the second 28-day cycle, magrolimab is administered at a dose of 15 mg/kg weekly (QW); and bevacizumab is administered at a dose of 5 mg/kg on days 1 and 15; and
c) for the third 28-day cycle, magrolimab is administered at a dose of 15 mg/kg Q2W; and bevacizumab is administered at a dose of 5 mg/kg on days 1 and 15.

90. The method of claim 46, wherein the subject is human.

91. A kit comprising one or more unitary doses of: (a) an agent that inhibits binding between CD47 and SIRPα; and (b) an agent that inhibits binding between vascular endothelial growth factor A (VEGFA) and one or more VEGFA cognate receptors.

92-114. (canceled)
